(19) United States
(12) Patent Application Publication
(10) Pub. No.: US 2023/0008090 A1
(43) Pub. Date: Jan. 12, 2023

(54) A NOVEL ANTI-CD3/ANTI-EGFR BISPECIFIC ANTIBODY AND USES THEREOF

(71) Applicant: WUXI BIOLOGICS IRELAND LIMITED, DUNDALK, CO LOUTH (IE)

(72) Inventors: Qin MEI, Shanghai (CN); Zhuozhi WANG, Shanghai (CN); Jing LI, Shanghai (CN); Jianqing XU, Shanghai (CN)

(21) Appl. No.: 17/777,596

(22) PCT Filed: Nov. 27, 2020
(86) PCT No.: PCT/CN2020/132157

§ 371 Date: May 17, 2022

(30) Foreign Application Priority Data
Nov. 29, 2019 (CN) PCT/CN2019/121869

(51) Int. Cl. C07K 16/28; A61P 35/00

(52) U.S. Cl. CPC C07K 16/2809; A61P 35/00; C07K 2317/31; C07K 2317/55; C07K 2317/53; C07K 2317/524; C07K 2317/526; C07K 2317/24

(57) ABSTRACT

Provided are bispecific antibodies against CD3 and EGFR, the nucleic acid molecules encoding the antibodies, expression vectors and host cells used for the expression of the antibodies. The antibodies provide a potent agent for the treatment of CD3-related and/or EGFR-related diseases via modulating immune functions.

### PRIORITY INFORMATION

The present application claims the benefits of PCT Application No. PCT/CN2019/121869 filed on Nov. 29, 2019, which is incorporated herein by reference as an entirety.

### SEQUENCE LISTING

The present application is filed with a Sequence Listing in electronic form. The entire contents of the Sequence Listing are hereby incorporated by reference.

### FIELD

This application generally relates to antibodies. More specifically, the application relates to bispecific antibodies against CD3 and EGFR, a method for preparing the same, and the use of the bispecific antibodies.

### BACKGROUND

The epidermal growth factor receptor (EGFR) is expressed in a wide range of human tissues and its over-expression is associated with a number of malignant tumors of epithelia cell origin, such as colorectal cancer, non-small cell lung cancer, head and neck cancer. Through binding to its ligand EGF, EGFR undergoes a transition from an inactive monomeric form to an active homodimer or heterodimer, and then induces tyrosine phosphorylation and downstream signal leading to tumor cells uncontrolled proliferation. Two EGFR-targeted antibodies, cetuximab (Erbitux) and panitumumab (Vectibix), have been approved by the US Food and Drug Administration for the treatment of colon cancers and head and neck cancers.

According to clinical statistics, varying anti-tumor efficacy was observed with existing EGFR-specific therapeutic antibodies, and refractory or relapsed EGFR-expressing tumors proved disappointing clinical outcomes. Especially patients with EGFR mutation, KRAS, BRAF mutations, which comprise approximately 40-50% of colorectal cancers, do not respond to anti-EGFR therapeutic antibodies.

T cells play an extremely pivotal role in eliminating tumor cells and controlling tumor growth by broad range of cytotoxic effects, such as proteolytic enzymes (granzymes) and pore-forming proteins (perforin) after T cell recognition and activation against tumor cells. Monoclonal antibody (such as OKT3) targeting T cells, particularly targeting CD3 molecular of the T cells has become increasingly promising immuno-therapeutical approach, like CD3-based BITE monoclonal antibodies and CAR-T as well as CD3 mediated bi-specific antibodies. Mouse monoclonal antibodies specific for human CD3, such as OKT3 (Kung et al., Science, 206: 347-9 (1979)), were the first generation CD3 antibodies developed for treatment. Although OKT3 has strong immunosuppressive potency, its clinical use was hampered by serious side effects linked to its immunogenic and mitogenic potentials (Chatenoud, Nature Reviews, 3:123-132 (2003)). OKT3 induced an anti-globulin response, promoting its own rapid clearance and neutralization (Chatenoud et al., Eur. J. Immunol., 137:830-8 (1982)). In addition, OKT3 induced T-cell proliferation and cytokine production in vitro, and led to a large-scale release of cytokine in vivo (Hirsch et al., J. Immunol, 142: 737-43 (1989)). Such serious side effects limited the more widespread use of OKT3 in transplantation as well as the extension of its use to other clinical fields such as autoimmunity. A bispecific antibody targeting CD3 (T cell activation) and EGFR may provide an alternative therapeutic regimen for the patients with refractory or relapsed EGFR-expressing tumors.

Therefore, there are urgent needs to address this high unmet clinical need by developing a bispecific antibody targeting CD3 and EGFR as an alternative immunotherapy strategy for the patients with the tumor resistant or refractory to EGFR monoclonal antibodies, such as cetuximab (Erbitux) and panitumumab (Vectibix) treatment.

### SUMMARY

The present disclosure has an objective to establish a bispecific antibody (BsAb) combining EGFR and CD3 dual binding activity which can provide promising treatment efficacy. The BsAb can bind EGFR and block the interaction between EGFR and its ligands and redirect cytotoxic T cells to EGFR-expressing tumor cells (both tumor cells expressing wild type EGFR and tumor cells expressing mutated EGFR variants), and subsequently destroy the tumor cells more specifically and efficiently with less off-tumor toxicity.

The present disclosure, in a broad sense, is directed to an anti-CD3 and anti-EGFR bispecific antibody with improved efficacy, compounds, compositions and articles of manufacture comprising the bispecific antibody, methods for manufacturing the antibody. The benefits provided by the present disclosure are broadly applicable in the field of antibody therapeutics and diagnostics and may be used in conjunction with antibodies that react with a variety of targets.

The present disclosure provides a bispecific antibody against CD3 and EGFR. It also provides isolated nucleotide sequence encoding the anti-CD3/anti-EGFR antibody, expression vectors and host cells used for the expression of bispecific antibody. The present disclosure further provides the methods for preparing the anti-CD3/anti-EGFR antibody, validating its functions in vivo and in vitro. The bispecific antibody of the present disclosure provides a very potent agent for preventing or treating diseases comprising proliferative disorders, immune disorders, or infections. In some embodiments, the diseases are CD3-related and/or EGFR-related diseases.

In some aspects, the present disclosure provides a bispecific antibody or the antigen-binding portion thereof, comprising a first antigen-binding site that specifically binds to CD3 and a second antigen-binding site that specifically binds to an antigen different from CD3.

In some aspects, the antigen different from CD3 is EGFR.

In some aspects, the present disclosure provides a bispecific antibody or the antigen-binding portion thereof, comprising a first antigen-binding moiety associated with a second antigen-binding moiety, wherein:
the first antigen-binding moiety is a CD3 antigen binding moiety and comprises:
   a first heavy chain variable domain (VH1) of a first antibody operably linked to an antibody heavy chain CH1 domain, and
   a first light chain variable domain (VL1) of the first antibody operably linked to an antibody light chain constant (CL) domain,
the second antigen-binding moiety is an EGFR antigen binding moiety and comprises:
   a first polypeptide comprising, from N-terminus to C-terminus, a second heavy chain variable domain (VH2) of a second antibody operably linked to a first T cell receptor (TCR) constant region (C1), and
   a second polypeptide comprising, from N-terminus to C-terminus, a second light chain variable domain (VL2) of the second antibody operably linked to a second TCR constant region (C2),
   wherein:
   C1 and C2 are capable of forming a dimer via a non-native interchain disulphide bond which is capable of stabilizing the dimer,
   and
wherein:
   the CD3 antigen binding moiety is derived from an anti-CD3 antibody, and comprises:
      a) a heavy chain CDR1 comprising or consisting of an amino acid sequence represented by SEQ ID NO: 1,
      b) a heavy chain CDR2 comprising or consisting of an amino acid sequence represented by SEQ ID NO: 2,
      c) a heavy chain CDR3 comprising or consisting of an amino acid sequence represented by SEQ ID NO: 3,
      d) a light chain CDR1 comprising or consisting of an amino acid sequence represented by SEQ ID NO: 4,
      e) a light chain CDR2 comprising or consisting of an amino acid sequence represented by SEQ ID NO: 5, and
      f) a light chain CDR3 comprising or consisting of an amino acid sequence represented by SEQ ID NO: 6,
   and
   the EGFR antigen binding moiety is derived from an anti-EGFR antibody and comprises:
      a) a heavy chain CDR1 comprising or consisting of an amino acid sequence represented by SEQ ID NO: 7,
      b) a heavy chain CDR2 comprising or consisting of an amino acid sequence represented by SEQ ID NO: 8,
      c) a heavy chain CDR3 comprising or consisting of an amino acid sequence represented by SEQ ID NO: 9,
      d) a light chain CDR1 comprising or consisting of an amino acid sequence represented by SEQ ID NO: 10,
      e) a light chain CDR2 comprising or consisting of an amino acid sequence represented by SEQ ID NO: 11, and
      f) a light chain CDR3 comprising or consisting of an amino acid sequence represented by SEQ ID NO: 12.

In some embodiments, the present disclosure provides a bispecific antibody or an antigen-binding portion thereof, comprising a CD3 antigen binding moiety and an EGFR antigen binding moiety, wherein:

the CD3 antigen binding moiety comprises a Fab comprising a first VH (VH1) of an anti-CD3 antibody operably linked to a heavy chain CH1 constant region domain, and a first VL (VL1) of the anti-CD3 antibody operably linked to a light chain constant region (CL), and

the EGFR antigen binding moiety comprises a chimeric Fab comprising a second heavy chain variable domain (VH2) of an anti-EGFR antibody operably linked to a first T cell receptor (TCR) constant region (C1), and a second light chain variable domain (VL2) of the anti-EGFR antibody operably linked to a second TCR constant region (C2), and wherein C1 and C2 are capable of forming a dimer via a non-native interchain disulphide bond which is capable of stabilizing the dimer,

wherein:

(A) the CD3 antigen binding moiety comprises:

a heavy chain CDR1 comprising or consisting of an amino acid sequence represented by SEQ ID NO: 1,

a heavy chain CDR2 comprising or consisting of an amino acid sequence represented by SEQ ID NO: 2,

a heavy chain CDR3 comprising or consisting of an amino acid sequence represented by SEQ ID NO: 3,

a light chain CDR1 comprising or consisting of an amino acid sequence represented by SEQ ID NO: 4,

a light chain CDR2 comprising or consisting of an amino acid sequence represented by SEQ ID NO: 5, and

a light chain CDR3 comprising or consisting of an amino acid sequence represented by SEQ ID NO: 6,

and

(B) the EGFR antigen binding moiety comprises:

a heavy chain CDR1 comprising or consisting of an amino acid sequence represented by SEQ ID NO: 7,

a heavy chain CDR2 comprising or consisting of an amino acid sequence represented by SEQ ID NO: 8,

a heavy chain CDR3 comprising or consisting of an amino acid sequence represented by SEQ ID NO: 9,

a light chain CDR1 comprising or consisting of an amino acid sequence represented by SEQ ID NO: 10,

a light chain CDR2 comprising or consisting of an amino acid sequence represented by SEQ ID NO: 11, and

a light chain CDR3 comprising or consisting of an amino acid sequence represented by SEQ ID NO: 12.

In some embodiments, the first T cell receptor (TCR) constant region (C1 domain) comprises a TCRβ constant region comprising the amino acid sequence of SEQ ID NO: 29, and in one preferred embodiment, the C1 domain comprises or consists of a TCRβ constant region represented by SEQ ID NO: 29.

In some embodiments, the second T cell receptor (TCR) constant region (C2 domain) comprises a TCRα constant region comprising the amino acid sequence of SEQ ID NO: 30, and in one preferred embodiment, the C2 domain comprises or consists of a TCRα constant region represented by SEQ ID NO: 30.

In some embodiments, the bispecific antibody or the antigen-binding portion thereof disclosed herein further comprises an Fc region, wherein the Fc region is operably linked to the CH1 domain of the CD3 antigen binding moiety.

In some embodiments, the Fc region is a human Fc region, such as a human IgG Fc region, especially a human IgG4 or IgG1 Fc region. Preferably, the Fc region is human IgG4 Fc region containing mutations S228P, F234A and L235A.

In some embodiments, the present disclosure provides a bispecific antibody or an antigen-binding portion thereof, comprising a CD3 antigen binding moiety and an EGFR antigen binding moiety, wherein:

(A) the CD3 antigen binding moiety comprises:

a heavy chain CDR1 consisting of an amino acid sequence represented by SEQ ID NO: 1,

a heavy chain CDR2 consisting of an amino acid sequence represented by SEQ ID NO: 2,

a heavy chain CDR3 consisting of an amino acid sequence represented by SEQ ID NO: 3,

a light chain CDR1 consisting of an amino acid sequence represented by SEQ ID NO: 4,

a light chain CDR2 consisting of an amino acid sequence represented by SEQ ID NO: 5, and

a light chain CDR3 consisting of an amino acid sequence represented by SEQ ID NO: 6,

and

(B) the EGFR antigen binding moiety comprises:

a heavy chain CDR1 consisting of an amino acid sequence represented by SEQ ID NO: 7,

a heavy chain CDR2 consisting of an amino acid sequence represented by SEQ ID NO: 8,

a heavy chain CDR3 consisting of an amino acid sequence represented by SEQ ID NO: 9,

a light chain CDR1 consisting of an amino acid sequence represented by SEQ ID NO: 10,

a light chain CDR2 consisting of an amino acid sequence represented by SEQ ID NO: 11, and

a light chain CDR3 consisting of an amino acid sequence represented by SEQ ID NO: 12.

In certain embodiments, the CD3 antigen binding moiety of the bispecific antibody is derived from an anti-CD3 antibody and comprises:

(i) a heavy chain variable domain (VH1) sequence comprising or consisting of SEQ ID NO: 13, and

(ii) a light chain variable domain (VL1) sequence comprising or consisting of SEQ ID NO: 14.

In certain embodiments, the EGFR antigen binding moiety of the bispecific antibody is derived from an anti-EGFR antibody and comprises:

(i) a heavy chain variable domain (VH2) sequence comprising or consisting of SEQ ID NO: 15, and

(ii) a light chain variable domain (VL2) sequence comprising or consisting of SEQ ID NO: 16.

In certain embodiments, the bispecific antibody or the antigen-binding portion thereof comprises a first antigen-binding moiety associated with a second antigen-binding moiety, wherein:

the first antigen-binding moiety is a CD3 binding moiety comprising:

(i) a heavy chain variable domain (VH1) sequence comprising or consisting of SEQ ID NO: 13, and

(ii) a light chain variable domain (VL1) sequence comprising or consisting of SEQ ID NO: 14;

and

the second antigen-binding moiety is an EGFR binding moiety comprising:

(i) a heavy chain variable domain (VH2) sequence comprising or consisting of SEQ ID NO: 15, and

(ii) a light chain variable domain (VL2) sequence comprising or consisting of SEQ ID NO: 16.

In some embodiments, the CD3 binding moiety comprises:

(i) a heavy chain variable domain (VH1) sequence having at least 85% sequence identity, for example, 86%, 87%, 88%, 89%, 90%, 91%, 92%, 93%, 94%, 95%, 96%, 97%, 98%, 99%, or even 100% sequence identity to SEQ ID NO: 13 and at the same time maintaining the binding specificity to CD3; and

(ii) a light chain variable domain (VL1) sequence having at least 85% sequence identity, for example, 86%, 87%, 88%, 89%, 90%, 91%, 92%, 93%, 94%, 95%, 96%, 97%, 98%, 99%, or even 100% sequence identity to SEQ ID NO: 14 and at the same time maintaining the binding specificity to CD3.

In some embodiments, the EGFR binding moiety comprises:

(i) a heavy chain variable domain (VH2) sequence having at least 85% sequence identity, for example, 86%, 87%, 88%, 89%, 90%, 91%, 92%, 93%, 94%, 95%, 96%, 97%, 98%, 99%, or even 100% sequence identity to SEQ ID NO: 15 and at the same time maintaining the binding specificity to EGFR; and

(ii) a light chain variable domain (VL2) sequence having at least 85% sequence identity, for example, 86%, 87%, 88%, 89%, 90%, 91%, 92%, 93%, 94%, 95%, 96%, 97%, 98%, 99%, or even 100% sequence identity to SEQ ID NO: 16 and at the same time maintaining the binding specificity to EGFR.

In certain embodiments, the bispecific antibody or the antigen-binding portion thereof comprises a first antigen-binding moiety associated with a second antigen-binding moiety, wherein:

the first antigen-binding moiety is a CD3 binding moiety comprising:

(i) a heavy chain variable domain (VH1) sequence consisting of SEQ ID NO: 13, and

(ii) a light chain variable domain (VL1) sequence consisting of SEQ ID NO: 14;

and

the second antigen-binding moiety is an EGFR binding moiety comprising:

(i) a heavy chain variable domain (VH2) sequence consisting of SEQ ID NO: 15, and

(ii) a light chain variable domain (VL2) sequence consisting of SEQ ID NO: 16.
In some embodiments, the bispecific antibody or the antigen-binding portion thereof comprises four polypeptide chains:

i) a first heavy chain represented by VH1-CH1-Hinge1-CH2-CH3;

ii) a first light chain represented by VL1-CL;

iii) a second heavy chain represented by VH2-C1-Hinge2-CH2-CH3, and

iv) a second light chain represented by VL2-C2;

wherein VH1-CH1 portion of i) and VL1-CL form an anti-CD3 arm (referred to as T3, see FIG. 1), and VH2-C1 of iii) and VL2-C2 form an anti-EGFR arm (referred to as U1, see FIG. 1);

wherein C1 and C2 are capable of forming a dimer comprising at least one non-native inter-chain bond, and the two hinge regions and/or the two CH3 domains are capable of forming one or more inter-chains bond that can facilitate dimerization.

In certain embodiments, the bispecific antibody or the antigen-binding portion thereof comprises four polypeptide chains:

i) a first heavy chain represented by SEQ ID NO: 23 or an amino acid sequence having at least 85%, for example, 86%, 87%, 88%, 89%, 90%, 91%, 92%, 93%, 94%, 95%, 96%, 97%, 98%, 99%, or even 100% sequence identity to SEQ ID NO: 23 and at the same time maintaining the binding specificity to CD3;

ii) a first light chain represented by SEQ ID NO: 22 or an amino acid sequence having at least 85%, for example, 86%, 87%, 88%, 89%, 90%, 91%, 92%, 93%, 94%, 95%, 96%, 97%, 98%, 99%, or even 100% sequence identity to SEQ ID NO: 22 and at the same time maintaining the binding specificity to CD3;

iii) a second heavy chain represented by SEQ ID NO: 24 or an amino acid sequence having at least 85%, for example, 86%, 87%, 88%, 89%, 90%, 91%, 92%, 93%, 94%, 95%, 96%, 97%, 98%, 99%, or even 100% sequence identity to SEQ ID NO: 24 and at the same time maintaining the binding specificity to EGFR, and

iv) a second light chain represented by SEQ ID NO: 21 or an amino acid sequence having at least 85%, for example, 86%, 87%, 88%, 89%, 90%, 91%, 92%, 93%, 94%, 95%, 96%, 97%, 98%, 99%, or even 100% sequence identity to SEQ ID NO: 21 and at the same time maintaining the binding specificity to EGFR.

In certain embodiments, the bispecific antibody or the antigen-binding portion thereof comprises four polypeptide chains:

i) a first heavy chain represented by SEQ ID NO: 23;

ii) a first light chain represented by SEQ ID NO: 22;

iii) a second heavy chain represented by SEQ ID NO: 24, and

iv) a second light chain represented by SEQ ID NO: 21.

In certain embodiments, the bispecific antibody or the antigen-binding portion thereof consists of four polypeptide chains:

i) a first heavy chain represented by SEQ ID NO: 23;

ii) a first light chain represented by SEQ ID NO: 22;

iii) a second heavy chain represented by SEQ ID NO: 24, and

iv) a second light chain represented by SEQ ID NO: 21.

In some embodiments, the CD3 and EGFR antigens can be derived from cynomolgus monkey, or human CD3 and EGFR proteins, among others. Preferably, the CD3 and EGFR proteins are human CD3 and EGFR proteins. In a preferred embodiment, the above-described antibodies can specifically bind to human CD3 and EGFR proteins simultaneously.

In some embodiments, the first T cell receptor (TCR) constant region (C1 domain) comprises a TCRβ constant region comprising the amino acid sequence of SEQ ID NO: 29, and in one preferred embodiment, the C1 domain comprises or consists of a TCRβ constant region represented by SEQ ID NO: 29.

In some embodiments, the second T cell receptor (TCR) constant region (C2 domain) comprises a TCRα constant region comprising the amino acid sequence of SEQ ID NO: 30, and in one preferred embodiment, the C2 domain comprises or consists of a TCRα constant region represented by SEQ ID NO: 30.

In some embodiments, the C1 domain comprises the amino acid sequence of SEQ ID NO: 29, and the C2 domain comprises the amino acid sequence of SEQ ID NO: 30.

In some embodiments, the C1 domain consists of the amino acid sequence of SEQ ID NO: 29, and the C2 domain consists of the amino acid sequence of SEQ ID NO: 30.

In some embodiments, the bispecific antibody or the antigen-binding portion thereof disclosed herein further comprises an Fc region, wherein the Fc region is operably linked to the CH1 domain of the CD3 antigen binding moiety.

In some embodiments, the Fc region is a human Fc region, such as a human IgG Fc region, especially a human IgG4 or IgG1 Fc region. Preferably, the Fc region is human IgG4 Fc region containing mutations S228P, F234A and L235A.

In some embodiments, the bispecific antibody or the antigen-binding portion thereof as disclosed herein is a humanized antibody.

In some aspects, the present disclosure provides a bispecific antibody or the antigen-binding portion thereof having one or more of the following properties:

(a) specifically binding to human CD3 and EGFR protein simultaneously with a high affinity;

(b) specifically binding to human CD3 and/or cyno CD3 protein;

(c) specifically binding to human EGFR and/or cyno EGFR protein;

(d) capable of inducing potent T cell activation in the presence of EGFR-expression tumor cells compared to anti-CD3 antibodies, anti-EGFR antibodies, a combination thereof, and other bispecific antibodies targeting CD3 and EGFR;

(e) providing good thermal stability and being stable in human serum; and

(f) providing superior anti-tumor effect compared to anti-CD3 antibodies, anti-EGFR antibodies, a combination thereof, and other bispecific antibodies targeting CD3 and EGFR.

In some aspects, the present disclosure provides an isolated nucleic acid molecule, comprising a nucleic acid sequence encoding the bispecific antibody or the antigen-binding portion thereof as defined herein.

In some embodiments, the present disclosure provides an isolated nucleotide sequence encoding the heavy chain variable domain (VH1) of the CD3 binding moiety, an isolated nucleotide sequence encoding the light chain variable domain (VL1) of the CD3 binding moiety, an isolated nucleotide sequence encoding the heavy chain variable domain (VH2) of the EGFR binding moiety, and an isolated nucleotide sequence encoding the light chain variable domain (VL2) of the EGFR binding moiety.

In some embodiments, the isolated nucleotide sequence encoding the heavy chain variable domain (VH1) of the CD3 binding moiety is represented by SEQ ID NO: 17, and the isolated nucleotide sequence encoding the light chain variable domain (VL1) of the CD3 binding moiety is represented by SEQ ID NO: 18.

In some embodiments, the isolated nucleotide sequence encoding the heavy chain variable domain (VH2) of the EGFR binding moiety is represented by SEQ ID NO: 19, and the isolated nucleotide sequence encoding the light chain variable domain (VL2) is represented by SEQ ID NO: 20.

In some embodiments, the present disclosure provides an isolated nucleotide sequence encoding the heavy chain of the CD3 binding moiety, wherein the isolated nucleotide sequence encoding the heavy chain of the CD3 binding moiety comprises or consists of:

(A) a nucleic acid sequence that encodes the heavy chain as set forth in SEQ ID NO: 23;

(B) a nucleic acid sequence as set forth in SEQ ID NO: 27; or

(C) a nucleic acid sequence that hybridized under high stringency conditions to the complementary strand of the nucleic acid sequence of (A) or (B).

In some embodiments, the present disclosure provides an isolated nucleotide sequence encoding the light chain of the CD3 binding moiety, wherein the isolated nucleotide sequence encoding the light chain of the CD3 binding moiety comprises or consists of:

(A) a nucleic acid sequence that encodes the heavy chain as set forth in SEQ ID NO: 22;

(B) a nucleic acid sequence as set forth in SEQ ID NO: 26; or

(C) a nucleic acid sequence that hybridized under high stringency conditions to the complementary strand of the nucleic acid sequence of (A) or (B).

In some embodiments, the present disclosure provides an isolated nucleotide sequence encoding the heavy chain of the EGFR binding moiety, wherein the isolated nucleotide sequence encoding the heavy chain of the EGFR binding moiety comprises or consists of:

(A) a nucleic acid sequence that encodes the heavy chain as set forth in SEQ ID NO: 24;

(B) a nucleic acid sequence as set forth in SEQ ID NO: 28; or

(C) a nucleic acid sequence that hybridized under high stringency conditions to the complementary strand of the nucleic acid sequence of (A) or (B).

In some embodiments, the present disclosure provides an isolated nucleotide sequence encoding the light chain of the EGFR binding moiety, wherein the isolated nucleotide sequence encoding the light chain of the EGFR binding moiety comprises or consists of:

(A) a nucleic acid sequence that encodes the heavy chain as set forth in SEQ ID NO: 21;

(B) a nucleic acid sequence as set forth in SEQ ID NO: 25; or

(C) a nucleic acid sequence that hybridized under high stringency conditions to the complementary strand of the nucleic acid sequence of (A) or (B).

In some aspects, the present disclosure provides a vector comprising the nucleic acid molecule as defined herein.

In some aspects, the present disclosure provides a host cell comprising the isolated nucleic acid molecule or the vector as disclosed herein.

In some embodiments, the host cell may be selected from, but not limited to, cells from prokaryotes or eukaryotic microbes, such as bacteria cells (e.g., eubacteria, such as Gram-negative or Gram-positive organisms, for example, Enterobacteriaceae such as Escherichia, e.g., E. coli), and fungal cells (e.g., yeast cells, filamentous fungi cells, etc.), plant cells or animal cells.

In some aspects, the present disclosure provides a pharmaceutical composition comprising the bispecific antibody or the antigen-binding portion thereof as defined herein and a pharmaceutically acceptable carrier.

In some aspects, the present disclosure provides a method for producing the bispecific antibody or the antigen-binding portion thereof as defined herein, comprising the steps of:
expressing the antibody or the antigen-binding portion thereof in the host cell as described above; and
isolating the antibody or antigen-binding portion thereof from the host cell.

In some embodiments, the present disclosure provides a method for producing the bispecific antibody or the antigen-binding portion thereof as defined herein, comprising the steps of:
expressing the antibody or the antigen-binding portion thereof in a host cell comprising the isolated nucleic acid molecule which comprises a nucleic acid sequence encoding the bispecific antibody or the antigen-binding portion thereof as defined herein, and
isolating the antibody or antigen-binding portion thereof from the host cell.

In some embodiments, the present disclosure provides a method for producing the bispecific antibody or the antigen-binding portion thereof as defined herein, comprising the steps of:
expressing the antibody or the antigen-binding portion thereof in a host cell comprising an vector which comprises an isolated nucleic acid molecule, wherein the isolated nucleic acid molecule comprises a nucleic acid sequence encoding the bispecific antibody or the antigen-binding portion thereof as defined herein, and
isolating the antibody or antigen-binding portion thereof from the host cell.

In some aspects, the present disclosure provides a method for modulating an immune response in a subject, comprising administering an effective amount of the bispecific antibody or the antigen-binding portion thereof or the pharmaceutical composition as defined herein to the subject.

In some aspects, the present disclosure provides a method for inhibiting growth of tumor cells in a subject, comprising administering an effective amount of the bispecific antibody or the antigen-binding portion thereof or the pharmaceutical composition as defined herein to the subject.

In some aspects, the present disclosure provides a method for preventing or treating CD3-related and/or EGFR-related diseases comprising proliferative disorders, immune disorders, or infections in a subject, comprising administering an effective amount of the bispecific antibody or the antigen-binding portion thereof or the pharmaceutical composition as defined herein to the subject.

In some embodiments, the proliferative disorder is cancer, such as colon cancer, lung cancer, liver cancer, cervical cancer, breast cancer, ovarian cancer, pancreatic cancer, melanoma, glioblastoma, prostate cancer, esophageal cancer, or gastric cancer.

In some embodiments, the infection is a chronic infection.

In some embodiments, the bispecific antibody or antigen-binding portion thereof as defined herein may be administered in combination with a chemotherapeutic agent, radiation and/or other agents for use in cancer immunotherapy.

In some aspects, the present disclosure provides the bispecific antibody or the antigen-binding portion thereof for use

i) in the modulation of immune responses, such as restoring T cell activity;

ii) in enhancing T cell activation in the presence of EGFR-expression tumor cells; and/or

iii) in stimulating an immune response or function, such as boosting the immune response against cancer cells.

In some aspects, the present disclosure provides the bispecific antibody or the antigen-binding portion thereof as defined herein for use in treating or preventing CD3-related and/or EGFR-related disease including proliferative disorders (such as cancers), immune disorders, or infections.

In some aspects, the present disclosure provides the bispecific antibody or the antigen-binding portion thereof as defined herein for use in diagnosing CD3-related and/or EGFR-related disease including proliferative disorders (such as cancers), immune disorders, or infections. In some aspects, the present disclosure provides use of the bispecific antibody or the antigen-binding portion thereof of as defined herein in the manufacture of a medicament for modulating an immune response or inhibiting growth of tumor cells in a subject.

In some aspects, the present disclosure provides use of the bispecific antibody or the antigen-binding portion thereof of as defined herein in the manufacture of a medicament for treating or preventing CD3-related and/or EGFR-related disease including proliferative disorders (such as cancers), immune disorders, or infections.

In some aspects, the present disclosure provides a kit which comprises a container comprising the bispecific antibody or the antigen-binding portion thereof as defined herein.

In some embodiments, the kit is used for treating or diagnosing CD3-related and/or EGFR-related disease including proliferative disorders (such as cancers), immune disorders or infections.

In some embodiments, the CD3-related and/or EGFR-related disease is EGFR-related solid tumor. In preferred embodiments, the EGFR-related solid tumor is characterized by high EGFR expression.

In some embodiments, the kit may further comprise an instruction of use, and a package that separates each of the components in the kit.

In an embodiment, the kit further comprises an instruction for using the bispecific antibody for detection, diagnosis, prognosis, prevention or treatment of a CD3-related and/or EGFR-related disease in a subject.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Further, the contents of all references, patents and published patent applications cited throughout this application are incorporated herein in entirety by reference.

### BRIEF DESCRIPTION OF THE FIGURES

FIG. 1 is schematic representation of W3448-T3U1.E17R-1.uIgG4V9, wherein T3 represents anti-CD3 arm, and U1 represents anti-EGFR arm.

FIG. 2 shows the SDS-PAGE result of W3448-T3U1.E17R-1.uIgG4V9. M: Protein marker; Lane 1: Non-reduced; Lane 2: Reduced.

FIG. 3 shows SEC-HPLC chromatogram of W3448-T3U1.E17R-1.uIgG4V9.

FIG. 4 shows the binding activity of W3448-T3U1.E17R-1.uIgG4V9 to human CD3 (A) and EGFR (B) as measured by FACS using Jurkat.2B8 and A431 cell lines, respectively.

FIG. 5 shows the binding activity of W3448-T3U1.E17R-1.uIgG4V9 to cynomolgus monkey CD3 (A) and EGFR (B) as measured by FACS using cynomolgus monkey PBMC and EGFR-expressing stable CHOK1 cell lines, respectively.

FIG. 6 shows the binding affinity of W3448-T3U1.E17R-1.uIgG4V9 to human CD3 (A) and EGFR (B) as tested on Jurkat.2B8 and A431 cells, respectively, by flow cytometry.

FIG. 7 shows the bridging binding activity of W3448-T3U1.E17R-1.uIgG4V9 to CD3 and EGFR-expressing cells as tested by using pre-labeled Jurkat.2B8 and A431 cells via flow cytometry.

FIG. 8 shows the results of human T cell activation by W3448-T3U1.E17R-1.uIgG4V9 on tumor cells A431 (A, high EGFR expression) and HT-29 (B, medium EGFR expression), wherein HCC1419 cells (negative EGFR expression) were used as a control.

FIG. 9 shows the results of cynomolgus monkey T cell activation by W3448-T3U1.E17R-1.uIgG4V9 on tumor cells A431 (A) and HT-29 (B), wherein HCC1419 cells (negative EGFR expression) were used as a control.

FIG. 10 shows the cytotoxicity activity of W3448-T3U1.E17R-1.uIgG4V9 on tumor cells A431 (A), HT-29 (B), MCF-7 cells (C) and HCC1419 cells (D).

FIG. 11 shows the ADCC and CDC ability of W3448-T3U1.E17R-1.uIgG4V9 on Jurkat. 2B8 (A and C) and A431 cells (B and D).

FIG. 12 shows the thermal stability of W3448-T3U1.E17R-1.uIgG4V9 as measured by differential scanning fluorometry (DSF).

FIG. 13 shows the serum stability of W3448-T3U1.E17R-1.uIgG4V9.

FIG. 14 shows relative body weight change post administration tested in a mice model of human PBMC-HT29.

FIG. 15 shows the percentage of peripheral human CD3 (A) and the percentage of terminal human CD3 (B) in the in vivo anti-tumor efficacy study of W3448-T3U1.E17R-1.uIgG4V9 in human PBMC-HT29 model. In (A) and (B), each group of bars consist of 4 bars, from left to right, indicating the results from isotype control 0.3 mg/kg; Panitumumab, 0.3 mg/kg; W3448-T3U1.E17R-1.uIgG4V9, 0.3 mg/kg; and W3448-T3U1.E17R-1.uIgG4V9, 0.08 mg/kg, respectively.

FIG. 16 shows the tumor growth traced post administration tested in a mice model of human PBMC-HT29.

FIG. 17 shows relative animal body weight change post administration. Isotype (0.1 mg/kg) is used as negative control, and Panitumumab (0.1 mg/kg) is used as positive control.

FIG. 18 shows the tumor growth monitored post administration. Isotype (0.1 mg/kg) is used as negative control, and Panitumumab (0.1 mg/kg) is used as positive control.

FIG. 19 shows the results of W3448-T3U1.E17R-1.uIgG4V9 concentration in cynomolgus monkeys serum in a single dose PK study.

FIG. 20 shows detection results of the CD4+ and CD8+ T cells post administration.

FIG. 21 shows the effect of W3448-T3U1.E17R-1.uIgG4V9 on cytokines (i.e., IL-2, INF-7, TNF, IL-4, IL-5 and IL-6) release post administration.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art. Moreover, any section headings used herein are for organizational purposes only and are not to be construed as limiting the subject matter described.

### Definitions

In order to better understand the disclosure, the definitions and explanations of the relevant terms are provided as follows.

The terms “polypeptide,” “peptide,” and “protein” are used interchangeably herein to refer to a polymer of amino acid residues, or an assembly of multiple polymers of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an alpha-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. An alpha-carbon refers to the first carbon atom that attaches to a functional group, such as a carbonyl. A beta-carbon refers to the second carbon atom linked to the alpha-carbon, and the system continues naming the carbons in alphabetical order with Greek letters. Amino acid mimetics refers to chemical compounds that have a structure that is different from the general chemical structure of an amino acid, but that functions in a manner similar to a naturally occurring amino acid. The term “protein” typically refers to large polypeptides. The term “peptide” typically refers to short polypeptides. Polypeptide sequences are usually described as the left-hand end of a polypeptide sequence is the amino-terminus (N-terminus); the right-hand end of a polypeptide sequence is the carboxyl-terminus (C-terminus). “Polypeptide complex” as used herein refers to a complex comprising one or more polypeptides that are associated to perform certain functions. In certain embodiments, the polypeptides are immune-related.

The term “antibody” or “Ab,” herein is used in the broadest sense, which encompasses various antibody structures, including polyclonal antibodies, monospecific and multispecific antibodies (e.g. bispecific antibodies). A native intact antibody generally is a Y-shaped tetrameric protein comprising two heavy (H) and two light (L) polypeptide chains held together by covalent disulfide bonds and non-covalent interactions. Light chains of an antibody may be classified into κ and λ light chain. Heavy chains may be classified into μ, δ, γ, α and ε, which define isotypes of an antibody as IgM, IgD, IgG, IgA and IgE, respectively. In a light chain and a heavy chain, a variable region is linked to a constant region via a “J” region of about 12 or more amino acids, and a heavy chain further comprises a “D” region of about 3 or more amino acids. Each heavy chain consists of a heavy chain variable region (V_{H}) and a heavy chain constant region (C_{H}). A heavy chain constant region consists of 3 domains (C_{H}1, C_{H}2 and C_{H}3). Each light chain consists of a light chain variable region (V_{L}) and a light chain constant region (C_{L}). V_{H} and V_{L} region can further be divided into hypervariable regions (called complementary determining regions (CDR)), which are interspaced by relatively conservative regions (called framework region (FR)). Each V_{H} and V_{L} consists of 3 CDRs and 4 FRs in the following order: FR1, CDR1, FR2, CDR2, FR3, CDR3, FR4 from N-terminal to C-terminal. The variable region (V_{H} and V_{L}) of each heavy/light chain pair forms antigen binding sites, respectively. Distribution of amino acids in various regions or domains follows the definition in Kabat Sequences of Proteins of Immunological Interest (National Institutes of Health, Bethesda, Md. (1987 and 1991)), or Chothia & Lesk (1987) J. Mol. Biol. 196:901-917; Chothia et al., (1989) Nature 342:878-883. Antibodies may be of different antibody isotypes, for example, IgG (e.g., IgG1, IgG2, IgG3 or IgG4 subtype), IgA1, IgA2, IgD, IgE or IgM antibody.

The term “antigen-binding portion” or “antigen-binding fragment” of an antibody, which can be interchangeably used in the context of the application, refers to polypeptides comprising fragments of a full-length antibody, which retain the ability of specifically binding to an antigen that the full-length antibody specifically binds to, and/or compete with the full-length antibody for binding to the same antigen. Generally, see Fundamental Immunology, Ch. 7 (Paul, W., ed., the second edition, Raven Press, N.Y. (1989), which is incorporated herein by reference for all purposes. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein. In certain embodiments, an antigen-binding fragment of an antibody may contain at least one variable domain covalently linked to at least one constant domain. The variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule.

The term “variable domain” with respect to an antibody as used herein refers to an antibody variable region or a fragment thereof comprising one or more CDRs. Although a variable domain may comprise an intact variable region (such as HCVR or LCVR), it is also possible to comprise less than an intact variable region yet still retain the capability of binding to an antigen or forming an antigen-binding site.

The term “antigen-binding moiety” as used herein refers to an antibody fragment formed from a portion of an antibody comprising one or more CDRs, or any other antibody fragment that binds to an antigen but does not comprise an intact native antibody structure. Examples of antigen-binding moiety include, without limitation, a variable domain, a variable region, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a multispecific antibody, a camelized single domain antibody, a nanobody, a domain antibody, and a bivalent domain antibody. An antigen-binding moiety is capable of binding to the same antigen to which the parent antibody binds. In certain embodiments, an antigen-binding moiety may comprise one or more CDRs from a particular human antibody grafted to a framework region from one or more different human antibodies. For more and detailed formats of antigen-binding moiety are described in Spiess et al, 2015 (Supra), and Brinkman et al., mAbs, 9(2), pp. 182-212 (2017), which are incorporated herein by their entirety.

“Fab” with regard to an antibody refers to that portion of the antibody consisting of a single light chain (both variable and constant regions) associating to the variable region and first constant region of a single heavy chain by a disulfide bond. In certain embodiments, the constant regions of both the light chain and heavy chain are replaced with TCR constant regions.

“F(ab′)₂” refers to a dimer of Fab′.

A “fragment difficult (Fd)” with regard to an antibody refers to the amino-terminal half of the heavy chain fragment that can be combined with the light chain to form Fab.

“Fc” with regard to an antibody refers to that portion of the antibody consisting of the second (CH2) and third (CH3) constant regions of a first heavy chain bound to the second and third constant regions of a second heavy chain via disulfide bonding. The Fc portion of the antibody is responsible for various effector functions such as ADCC, and CDC, but does not function in antigen binding.

“Hinge region” in terms of an antibody includes the portion of a heavy chain molecule that joins the CH1 domain to the CH2 domain. This hinge region comprises approximately 25 amino acid residues and is flexible, thus allowing the two N-terminus antigen binding regions to move independently.

“CH2 domain” as used herein refers to includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat, E., et al., U.S. Department of Health and Human Services, (1983)).

The “CH3 domain” extends from the CH2 domain to the C-terminus of the IgG molecule and comprises approximately 108 amino acids. Certain immunoglobulin classes, e.g., IgM, further include a CH4 region.

“Fv” with regard to an antibody refers to the smallest fragment of the antibody to bear the complete antigen binding site. An Fv fragment consists of the variable domain of a single light chain bound to the variable domain of a single heavy chain. A number of Fv designs have been provided, including dsFvs, in which the association between the two domains is enhanced by an introduced disulphide bond; and scFvs can be formed using a peptide linker to bind the two domains together as a single polypeptide. Fvs constructs containing a variable domain of a heavy or light immunoglobulin chain associated to the variable and constant domain of the corresponding immunoglobulin heavy or light chain have also been produced. Fvs have also been multimerised to form diabodies and triabodies (Maynard et al., Annu Rev Biomed Eng 2 339-376 (2000)).

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)).

In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'S of the other moiety and form two binding sites which can target the same antigens (or epitopes) or different antigens (or epitopes).

In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

“ScFab” refers to a fusion polypeptide with a Fd linked to a light chain via a polypeptide linker, resulting in the formation of a single chain Fab fragment (scFab).

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g., a long flexible linker) and bound to two V_{L} moieties, respectively, via disulfide bridges. In some embodiments, dsFv-dsFv′ is bispecific in which each disulfide paired heavy and light chain has a different antigen specificity.

“Appended IgG” refers to a fusion protein with a Fab arm fused to an IgG to form the format of bispecific (Fab)₂-Fc. It can form a “IgG-Fab” or a “Fab-IgG”, with a Fab fused to the C-terminus or N-terminus of an IgG molecule with or without a connector. In certain embodiments, the appended IgG can be further modified to a format of IgG-Fab₄ (see, Brinkman et al., 2017, Supra).

The term “anti-CD3 antibody” or “CD3 antibody, as used herein, refers to an antibody, as defined herein, capable of binding to a CD3, for example, a human CD3.

The terms “CD3” and “CD3 protein” are used interchangeably herein. The CD3 protein is present in virtually all T cells. The CD3-TCR complex modulates T cell functions in both innate and adoptive immune response, as well as cellular and humoral immune functions. These include eliminating pathogenic organisms and controlling tumor growth by broad range of cytotoxic effects. The CD3 T-cell co-receptor is a protein complex composed of four distinct chains, a CD3gamma chain, a CD3delta chain, and two CD3epsilon chains. The four chains associate with a molecule known as T-cell receptor (TCR) and the zeta-chain to generate activation signal in T lymphocytes. The TCR, zetachain, and CD3 molecules compose the TCR complex, in which TCR as a subunit recognizes and binds to antigen, and CD3 as a subunit transfers and conveys the antigen stimulation to signaling pathway, and ultimately regulates T-cell activity. The term “CD3” may include human CD3, as well as variants, isoforms, and species homologs thereof. Accordingly, an antibody or antigen-binding portion thereof, as defined and disclosed herein, may also bind CD3 from species other than human, for example cynomolgus CD3.

The term “human CD3,” as used herein, refers to CD3 of human origin, such as the complete amino acid sequence of human CD3.

The term “cynomolgus CD3,” as used herein, refers to CD3 derived from cynomolgus monkey, such as the complete amino acid sequence of Rhesus macaque CD3.

The term “anti-EGFR antibody,” as used herein, refers to an antibody that specifically binds to EGFR. An “anti-EGFR antibody” may include monovalent antibodies with a single specificity. Exemplary anti-EGFR antibodies are described elsewhere herein.

The term “Epidermal growth factor receptor (EGFR)” is a 170 kilodalton (kDa) membrane-bound protein expressed on the surface of epithelial cells. EGFR is a member of the growth factor receptor family of protein tyrosine kinases, a class of cell cycle regulatory molecules. (W. J. Gullick et al., 1986, Cancer Res., 46:285-292). EGFR is activated when its ligand (either EGF or TGF-α) binds to the extracellular domain, resulting in autophosphorylation of the receptor's intracellular tyrosine kinase domain (S. Cohen et al., 1980, J. Biol. Chem., 255:4834-4842; A. B. Schreiber et al., 1983, J. Biol. Chem., 258:846-853).

EGFR is the protein product of a growth promoting oncogene, erbB or ErbB1, that is one member of a family, i.e., the ERBB family of protooncogenes, believed to play pivotal roles in the development and progression of many human cancers. In particular, increased expression of EGFR has been observed in breast, bladder, lung, head, neck and stomach cancer as well as glioblastomas.

The term “bivalent,” as used herein refers to an antibody or an antigen-binding fragment having two antigen-binding sites; the term “monovalent” refers to an antibody or an antigen-binding fragment having only one single antigen-binding site; and the term “multivalent” refers to an antibody or an antigen-binding fragment having multiple antigen-binding sites. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

The term “bispecific antigen-binding molecule” means a protein, polypeptide or molecular complex comprising at least a first antigen-binding domain (also referred to as a first antigen-binding site herein) and a second antigen-binding domain (also referred to as a second antigen-binding site herein). In some embodiment, the “bispecific antigen-binding molecule” is a “bispecific antibody”. Each antigen-binding domain within the bispecific antibody comprises at least one CDR that alone, or in combination with one or more additional CDRs and/or FRs, specifically binds to a particular antigen. In the context of the present disclosure, the first antigen-binding site specifically binds to a first antigen (e.g., CD3), and the second antigen-binding site specifically binds to a second, distinct antigen (e.g., EGFR).

The terms “anti-CD3/anti-EGFR antibody”, “anti-CD3/anti-EGFR bispecific antibody”, “antibody against CD3 and EGFR”, “anti-CD3×EGFR bispecific antibody”, “CD3×EGFR antibody”, as used herein interchangeably, refer to a bispecific antibody that specifically binds to CD3 and EGFR.

The term “monoclonal antibody” or “mAb”, as used herein, refer to a preparation of antibody molecules of single molecular composition. A monoclonal antibody displays a single binding specificity and affinity for a particular epitope.

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody,” as used herein, refers to an antibody in which the variable region sequences are derived from one species and the constant region sequences are derived from another species, such as an antibody in which the variable region sequences are derived from a mouse antibody and the constant region sequences are derived from a human antibody.

The term “recombinant antibody,” as used herein, refers to an antibody that is prepared, expressed, created or isolated by recombinant means, such as antibodies isolated from an animal that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

The term “spacer” as used herein refers to an artificial amino acid sequence having 1, 2, 3, 4 or 5 amino acid residues, or a length of between 5 and 15, 20, 30, 50 or more amino acid residues, joined by peptide bonds and are used to link one or more polypeptides. A spacer may or may not have a secondary structure. Spacer sequences are known in the art, see, for example, Holliger et al., Proc. Natl. Acad. Sci. USA 90:6444-6448 (1993); Poljak et al. Structure 2:1121-1123 (1994). Any suitable spacers known in the art can be used. For example, a useful spacer in the present disclosure may be rich in glycine and proline residues. Examples include spacers having a single or repeated sequence(s) composed of threonine/serine and glycine, such as TGGGG, GGGGS or SGGGG or its tandem repeats (e.g. 2, 3, 4, or more repeats).

The term “operably link” or “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “epitope” as used herein refers to the specific group of atoms or amino acids on an antigen to which an antibody binds. Epitopes can be formed both from contiguous amino acids (also called linear or sequential epitope) or noncontiguous amino acids juxtaposed by tertiary folding of a protein (also called configurational or conformational epitope). Epitopes formed from contiguous amino acids are typically arranged linearly along the primary amino acid residues on the protein and the small segments of the contiguous amino acids can be digested from an antigen binding with major histocompatibility complex (MHC) molecules or retained on exposure to denaturing solvents whereas epitopes formed by tertiary folding are typically lost on treatment with denaturing solvents. An epitope typically includes at least 3, and more usually, at least 5, about 7, or about 8-10 amino acids in a unique spatial conformation. Two antibodies may bind the same or a closely related epitope within an antigen if they exhibit competitive binding for the antigen. For example, if an antibody or antigen-binding moiety blocks binding of a reference antibody to the antigen by at least 85%, or at least 90%, or at least 95%, then the antibody or antigen-binding moiety may be considered to bind the same/closely related epitope as the reference antibody.

The term “specific binding” or “specifically binds” as used herein refers to a non-random binding reaction between two molecules, such as for example between an antibody and an antigen.

K_{D} is used to refer to the ratio of the dissociation rate to the association rate (k_{off}/kₒₙ), which may be determined by using any conventional method known in the art, including but not limited to surface plasmon resonance method, microscale thermophoresis method, HPLC-MS method and flow cytometry (such as FACS) method. In certain embodiments, the K_{D} value can be appropriately determined by using flow cytometry.

The term “fusion” or “fused” when used with respect to amino acid sequences (e.g. peptide, polypeptide, or protein) refers to combination of two or more amino acid sequences, for example by chemical bonding or recombinant means, into a single amino acid sequence that does not exist naturally. A fusion amino acid sequence may be produced by genetic recombination of two encoding polynucleotide sequences, and can be expressed by a method of introducing a construct containing the recombinant polynucleotides into a host cell.

The term “antigenic specificity” refers to a particular antigen or an epitope thereof that is selectively recognized by an antigen-binding molecule.

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “immunogenicity,” as used herein, refers to ability of stimulating the formation of specific antibodies or sensitized lymphocytes in organisms. It not only refers to the property of an antigen to stimulate a specific immunocyte to activate, proliferate and differentiate so as to finally generate immunologic effector substance such as antibody and sensitized lymphocyte, but also refers to the specific immune response that antibody or sensitized T lymphocyte can be formed in immune system of an organism after stimulating the organism with an antigen. Immunogenicity is the most important property of an antigen. Whether an antigen can successfully induce the generation of an immune response in a host depends on three factors, properties of an antigen, reactivity of a host, and immunization means.

The term “substitution” with regard to amino acid residue as used herein refers to naturally occurring or induced replacement of one or more amino acids with another in a peptide, polypeptide, or protein. Substitution in a polypeptide may result in diminishment, enhancement, or elimination of the polypeptide's function.

The term “mutation” or “mutated” with regard to an amino acid residue as used herein refers to substitution, insertion, or addition of an amino acid residue.

A native “T cell receptor” or a native “TCR” is a heterodimeric T cell surface protein which is associated with invariant CD3 chains to form a complex capable of mediating signal transduction. TCR belongs to the immunoglobulin superfamily, and is similar to a half antibody with a single heavy chain and a single light chain. a native TCR has an extracellular portion, a transmembrane portion, and an intracellular portion. The extracellular domain of a TCR has a membrane-proximal constant region and a membrane-distal variable region. In some embodiments disclosed herein, the bispecific antibodies comprise a soluble chimeric protein with the variable domains of an antibody and the constant domains of a TCR, wherein the subunits (such as alpha and beta domains) of the TCR constant domains are linked by an engineered disulfide bond.

The term “Ka,” as used herein, is intended to refer to the association rate of a particular antibody-antigen interaction, whereas the term “Kd” as used herein, is intended to refer to the dissociation rate of a particular antibody-antigen interaction. Kd values for antibodies can be determined using methods well established in the art. The term “K_{D}” as used herein, is intended to refer to the dissociation constant of a particular antibody-antigen interaction, which is obtained from the ratio of Kd to Ka (i.e., Kd/Ka) and is expressed as a molar concentration (M). A preferred method for determining the Kd of an antibody is by using surface plasmon resonance, preferably using a biosensor system such as a Biacore® system.

The term “high affinity” for an IgG antibody, as used herein, refers to an antibody having a K_{D} of 1×10⁻⁷ M or less, more preferably 5×10⁻⁸ M or less, even more preferably 1×10⁻⁸ M or less, even more preferably 5×10⁻⁹ M or less and even more preferably 1×10⁻⁹ M or less for a target antigen.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The term “compete for binding,” as used herein, refers to the interaction of two antibodies in their binding to a binding target. A first antibody competes for binding with a second antibody if binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not, be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s).

The ability of “inhibit binding,” as used herein, refers to the ability of an antibody or antigen-binding fragment thereof to inhibit the binding of two molecules (e.g., human CD3/EGFR and human anti-CD3/anti-EGFR antibody) to any detectable level. In certain embodiments, the binding of the two molecules can be inhibited at least 50% by the antibody or antigen-binding fragment thereof. In certain embodiments, such an inhibitory effect may be greater than 60%, greater than 70%, greater than 80%, or greater than 90%.

The term “epitope,” as used herein, refers to a portion on antigen that an immunoglobulin or antibody specifically binds to. “Epitope” is also known as “antigenic determinant”. Epitope or antigenic determinant generally consists of chemically active surface groups of a molecule such as amino acids, carbohydrates or sugar side chains, and generally has a specific three-dimensional structure and a specific charge characteristic. For example, an epitope generally comprises at least 3, 4, 5, 6, 7, 8, 9, 10, 11, 12, 13, 14 or 15 consecutive or non-consecutive amino acids in a unique steric conformation, which may be “linear” or “conformational”. See, for example, Epitope Mapping Protocols in Methods in Molecular Biology, Vol. 66, G. E. Morris, Ed. (1996). In a linear epitope, all the interaction sites between a protein and an interaction molecule (e.g., an antibody) are present linearly along the primary amino acid sequence of the protein. In a conformational epitope, the interaction sites span over amino acid residues that are separate from each other in a protein. Antibodies may be screened depending on competitiveness of binding to the same epitope by conventional techniques known by a person skilled in the art. For example, study on competition or cross-competition may be conducted to obtain antibodies that compete or cross-compete with each other for binding to antigens (e.g. RSV fusion protein). High-throughput methods for obtaining antibodies binding to the same epitope, which are based on their cross-competition, are described in an international patent application WO 03/48731.

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody,” as used herein, is intended to refer to an antibody that is substantially free of other antibodies having different antigenic specificities (e.g., an isolated antibody that specifically binds a CD3/EGFR protein is substantially free of antibodies that specifically bind antigens other proteins than CD3/EGFR). An isolated antibody that specifically binds a human CD3/EGFR protein may, however, have cross-reactivity to other antigens, such as CD3/EGFR proteins from other species. Moreover, an isolated antibody can be substantially free of other cellular material and/or chemicals.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells; cells from prokaryotes or eukaryotic microbes, such as bacteria cells (e.g., eubacteria, such as Gram-negative or Gram-positive organisms, for example, Enterobacteriaceae such as Escherichia, e.g., E. coli), and fungal cells (e.g., yeast cells, filamentous fungi cells, etc.); plant cells or animal cells such as insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

The term “transfection,” as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197.

The term “SPR” or “surface plasmon resonance,” as used herein, refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 5 and Jonsson, U., et al. (1993) Ann. Biol. Clin. 51:19-26; Jonsson, U., et al. (1991) Biotechniques 11:620-627; Johnsson, B., et al. (1995)J. Mol. Recognit. 8:125-131; and Johnnson, B., et al. (1991) Anal. Biochem. 198:268-277.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017-11-09.). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “subject” or “individual” or “animal” or “patient” as used herein refers to a human or non-human animal, including a mammal or a primate, in need of diagnosis, prognosis, amelioration, prevention, and/or treatment of a disease or condition. Mammalian subjects include humans, domestic animals, farm animals, and zoo, sports, or pet animals such as dogs, cats, guinea pigs, rabbits, rats, mice, horses, swine, cows, bears, and so on.

The term “effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and phagocytosis.

The term “antibody-dependent cell-mediated cytotoxicity” or “ADCC,” as used herein, refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγTRIII only, whereas monocytes express FcγTRI, FcγTRII and FcγTRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The term “complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202: 163 (1996), may be performed.

The term “cancer” as used herein refers to any medical condition characterized by malignant cell growth or neoplasm, abnormal proliferation, infiltration or metastasis, and includes both solid tumors and non-solid cancers (hematologic malignancies) such as leukemia. As used herein “solid tumor” refers to a solid mass of neoplastic and/or malignant cells. Examples of cancer or tumors include hematological malignancies, oral carcinomas (for example of the lip, tongue or pharynx), digestive organs (for example esophagus, stomach, small intestine, colon, large intestine, or rectum), peritoneum, liver and biliary passages, pancreas, respiratory system such as larynx or lung (small cell and non-small cell), bone, connective tissue, skin (e.g., melanoma), breast, reproductive organs (fallopian tube, uterus, cervix, testicles, ovary, or prostate), urinary tract (e.g., bladder or kidney), brain and endocrine glands such as the thyroid. In certain embodiments, the cancer is selected from ovarian cancer, breast cancer, head and neck cancer, renal cancer, bladder cancer, hepatocellular cancer, and colorectal cancer.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen. For instance, the “an effective amount,” when used in connection with treatment of CD3/EGFR-related diseases or conditions, refers to an antibody or antigen-binding portion thereof in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro AR, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

Bispecific Antibodies and Antigen-Binding Fragments Thereof

In certain embodiments, the antibodies and antigen-binding fragments thereof provided herein are bispecific. In some embodiments, the bispecific antibodies and antigen-binding fragments thereof provided herein have a first specificity for CD3, and a second specificity for a second antigen different from CD3 and whose blockade may produce a synergetic effect than blocking one antigen alone.

In certain embodiments, the second specificity is for a tumor associated antigen or an epitope thereof. The term “tumor associated antigen” refers to a target antigen expressed by tumor cells, however may be expressed by the cognate cell (or healthy cells) prior to transforming into a tumor. In some embodiments, the tumor associated antigens can be presented only by tumor cells and not by normal, i.e. non-tumor cells. In some other embodiments, the tumor associated antigens can be exclusively expressed on tumor cells or may represent a tumor specific mutation compared to non-tumor cells. In some other embodiments, the tumor associated antigens can be found in both tumor cells and non-tumor cells, but is overexpressed on tumor cells when compared to non-tumor cells or are accessible for antibody binding in tumor cells due to the less compact structure of the tumor tissue compared to non-tumor tissue. In some embodiments, the tumor associated antigen is located on the vasculature of a tumor.

Illustrative examples of a tumor associated antigen are LAG-3, CD10, CD19, CD20, CD22, CD21, CD22, CD25, CD30, CD33, CD34, CD37, CD44v6, CD45, CD133, Fms-like tyrosine kinase 3 (FLT-3, CD135), chondroitin sulfate proteoglycan 4 (CSPG4, melanoma-associated chondroitin sulfate proteoglycan), Epidermal growth factor receptor (EGFR), Her2neu, Her3, IGFR, IL3R, fibroblast activating protein (FAP), CDCP1, Derlin1, Tenascin, frizzled 1-10, the vascular antigens VEGFR2 (KDR/FLK1), VEGFR3 (FLT4, CD309), PDGFR-alpha (CD140a), PDGFR-beta (CD140b) Endoglin, CLEC14, Tem1-8, and Tie2. Further examples may include A33, CAMPATH-1 (CDw52), Carcinoembryonic antigen (CEA), Carboanhydrase IX (MN/CA IX), de2-7 EGFR, EGFRvIII, EpCAM, Ep-CAM, Folate-binding protein, G250, Fms-like tyrosine kinase 3 (FLT-3, CD135), c-Kit (CD117), CSF1R (CD115), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72.

In certain embodiments, the second specificity is for an infectious disease-associated antigen or an epitope thereof. Non-limiting examples of infectious disease-associated antigens include, e.g., an antigen that is expressed on the surface of a virus particle, or preferentially expressed on a cell that is infected with a virus, wherein the virus is selected from the group consisting of HIV, hepatitis (A, B or C), herpes virus (e.g., HSV-1, HSV-2, CMV, HAV-6, VZV, Epstein Barr virus), adenovirus, influenza virus, flavivirus, echovirus, rhinovirus, coxsackie virus, coronavirus, respiratory syncytial virus, mumps virus, rotavirus, measles virus, rubella virus, parvovirus, vaccinia virus, HTLV, dengue virus, papillomavirus, molluscum virus, poliovirus, rabies virus, JC virus, and arboviral encephalitis virus. Alternatively, the target antigen can be an antigen that is expressed on the surface of a bacterium, or preferentially expressed on a cell that is infected with a bacterium, wherein the bacterium is selected from the group consisting of chlamydia, rickettsia, mycobacteria, staphylococci, streptococci, pneumonococci, meningococci, gonococci, klebsiella, proteus, serratia, pseudomonas, legionella, diphtheria, salmonella, bacilli, cholera, tetanus, botulism, anthrax, plague, leptospira, and Lyme disease bacteria. In certain embodiments, the target antigen is an antigen that is expressed on the surface of a fungus, or preferentially expressed on a cell that is infected with a fungus, wherein the fungus is selected from the group consisting of Candida (albicans, krusei, glabrata, tropicalis, etc.), Crytococcus neoformans, Aspergillus (fumigatus, niger, etc.), Mucorales (mucor, absidia, rhizopus, etc.), Sporothrix schenkii, Blastomyces dermatitidis, Paracoccidioides brasiliensis, Coccidioides immitis, and Histoplasma capsulatum. In certain embodiments, the target antigen is an antigen that is expressed on the surface of a parasite, or preferentially expressed on a cell that is infected with a parasite, wherein the parasite is selected from the group consisting of Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondii, Nippostrongylus brasiliensis, Taenia crassiceps, and Brugia malayi. Non-limiting examples of specific pathogen-associated antigens include, e.g., HIV gp120, HIV CD4, hepatitis B glucoprotein L, hepatitis B glucoprotein M, hepatitis B glucoprotein S, hepatitis C E1, hepatitis C E2, hepatocyte-specific protein, herpes simplex virus gB, cytomegalovirus gB, and HTLV envelope protein.

According to certain exemplary embodiments, the present disclosure includes a bispecific antibody or the antigen-binding portion thereof, comprising a first antigen-binding site that specifically binds to CD3 and a second antigen-binding site that specifically binds to EGFR. Such antibodies may be referred to herein as, e.g., “anti-CD3/anti-EGFR,” or “anti-CD3/EGFR,” or “anti-CD3×EGFR” or “CD3×EGFR” bispecific antibodies, or other similar terminology.

The bispecific antibody of the disclosure binds to human CD3 and human EGFR with high affinity. The binding of an antibody of the disclosure to CD3 or EGFR can be assessed using one or more techniques well established in the art, for instance, ELISA. The binding specificity of an antibody of the disclosure can also be determined by monitoring binding of the antibody to cells expressing a CD3 protein or an EGFR protein, e.g., flow cytometry. For example, an antibody can be tested by a flow cytometry assay in which the antibody is reacted with a cell line that expresses human CD3, such as CHO cells that have been transfected to express CD3 on their cell surface. Additionally or alternatively, the binding of the antibody, including the binding kinetics (e.g., K_{D} value) can be tested in BIAcore binding assays. Still other suitable binding assays include ELISA or FACS assays, for example using a recombinant CD3 protein.

In some embodiments, the bispecific antibody or an antigen-binding portion thereof of the disclosure comprises a CD3 antigen binding moiety and an EGFR antigen binding moiety,

wherein the CD3 antigen binding moiety comprises a Fab comprising a first VH (VH1) of an anti-CD3 antibody operably linked to a heavy chain CHI constant region domain, and a first VL (VL1) of the anti-CD3 antibody operably linked to a light chain constant region (CL), and

the EGFR antigen binding moiety comprises a chimeric Fab comprising a second heavy chain variable domain (VH2) of an anti-EGFR antibody operably linked to a first T cell receptor (TCR) constant region (C1), and a second light chain variable domain (VL2) of the anti-EGFR antibody operably linked to a second TCR constant region (C2), and wherein C1 and C2 are capable of forming a dimer via a non-native interchain disulphide bond which is capable of stabilizing the dimer,

wherein:

(A) the CD3 antigen binding moiety comprises:

a heavy chain CDR1 comprising or consisting of an amino acid sequence represented by SEQ ID NO: 1,

a heavy chain CDR2 comprising or consisting of an amino acid sequence represented by SEQ ID NO: 2,

a heavy chain CDR3 comprising or consisting of an amino acid sequence represented by SEQ ID NO: 3,

a light chain CDR1 comprising or consisting of an amino acid sequence represented by SEQ ID NO: 4,

a light chain CDR2 comprising or consisting of an amino acid sequence represented by SEQ ID NO: 5, and

a light chain CDR3 comprising or consisting of an amino acid sequence represented by SEQ ID NO: 6,

and

(B) the EGFR antigen binding moiety comprises:

a heavy chain CDR1 comprising or consisting of an amino acid sequence represented by SEQ ID NO: 7,

a heavy chain CDR2 comprising or consisting of an amino acid sequence represented by SEQ ID NO: 8,

a heavy chain CDR3 comprising or consisting of an amino acid sequence represented by SEQ ID NO: 9,

a light chain CDR1 comprising or consisting of an amino acid sequence represented by SEQ ID NO: 10,

a light chain CDR2 comprising or consisting of an amino acid sequence represented by SEQ ID NO: 11, and

a light chain CDR3 comprising or consisting of an amino acid sequence represented by SEQ ID NO: 12.

In some embodiments, the bispecific antibody or the antigen-binding portion thereof has one or more of the following properties:

(a) specifically binding to human CD3 and EGFR protein simultaneously with a high affinity;

(b) specifically binding to human CD3 and/or cyno CD3 protein;

(c) specifically binding to human EGFR and/or cyno EGFR protein;

(d) capable of inducing potent T cell activation in the presence of EGFR-expression tumor cells compared to anti-CD3 antibodies, anti-EGFR antibodies, a combination thereof, and other bispecific antibodies targeting CD3 and EGFR;

(e) providing good thermal stability and being stable in human serum; and

(f) providing superior anti-tumor effect compared to anti-CD3 antibodies, anti-EGFR antibodies, a combination thereof, and other bispecific antibodies targeting CD3 and EGFR.

For instance, the bispecific antibody of the disclosure binds to a human CD3 protein with a K_{D} of 1×10⁻⁷ M or less, binds to a human CD3 protein with a K_{D} of 5×10⁻⁸ M or less, binds to a human CD3 protein with a K_{D} of 4×10⁻⁸ M or less, binds to a human CD3 protein with a K_{D} of 3×10⁻⁸ M or less, binds to a human CD3 protein with a K_{D} of 2×10⁻⁸ M or less, binds to a human CD3 protein with a K_{D} of 1×10⁻⁸ M or less, binds to a human CD3 protein with a K_{D} of 5×10⁻⁹ M or less, or binds to a human CD3 protein with a K_{D} of 4.70×10⁻⁹ M or less.

For instance, the bispecific antibody of the disclosure binds to a human EGFR protein with a K_{D} of 1×10⁻⁷ M or less, binds to a human EGFR protein with a K_{D} of 5×10⁻⁸ M or less, binds to a human EGFR protein with a K_{D} of 1×10⁻⁸ M or less, or binds to a human EGFR protein with a K_{D} of 6.20×10⁻⁹ M or less.

For instance, as studied in a tumor-bearing mice model, the bispecific antibody of the present disclosure achieved desirable tumor growth inhibition (TGI) as compared with Panitumumab (an anti-EGFR antibody), and unexpectedly achieved higher TGI at lower dose (e.g., 0.08 mg/kg body weight) than the TGI at a normal dose (e.g., 0.3 mg/kg body weight).

The First Antigen-Binding Moiety that Specifically Binds to CD3

The first antigen-binding moiety specifically binds to CD3, and thus, it is also referred to as the CD3 antigen binding moiety in the disclosure. The two terms can be used interchangeably.

The first antigen-binding moiety comprises a Fab comprising a first VH (VH1) of an anti-CD3 antibody operably linked to a heavy chain CH1 constant region domain, and a first VL (VL1) of the anti-CD3 antibody operably linked to a light chain constant region (CL).

In some embodiments, the first antigen-binding moiety comprises:

a) a heavy chain CDR1 comprising or consisting of an amino acid sequence selected from the group consisting of SEQ ID NO: 1 and an amino acid sequence different from SEQ ID NO: 1 by an amino acid addition, deletion or substitution of not more than 2 amino acids,
b) a heavy chain CDR2 comprising or consisting of an amino acid sequence selected from the group consisting of SEQ ID NO: 2 and an amino acid sequence different from SEQ ID NO: 2 by an amino acid addition, deletion or substitution of not more than 2 amino acids,
c) a heavy chain CDR3 comprising or consisting of an amino acid sequence selected from the group consisting of SEQ ID NO: 3 and an amino acid sequence different from SEQ ID NO: 3 by an amino acid addition, deletion or substitution of not more than 2 amino acids,
d) a light chain CDR1 comprising or consisting of an amino acid sequence selected from the group consisting of SEQ ID NO: 4 and an amino acid sequence different from SEQ ID NO: 4 by an amino acid addition, deletion or substitution of not more than 2 amino acids,
e) a light chain CDR2 comprising or consisting of an amino acid sequence selected from the group consisting of SEQ ID NO: 5 and an amino acid sequence different from SEQ ID NO: 5 by an amino acid addition, deletion or substitution of not more than 1 amino acids, and
f) a light chain CDR3 comprising or consisting of an amino acid sequence selected from the group consisting of SEQ ID NO: 6 and an amino acid sequence different from SEQ ID NO: 6 by an amino acid addition, deletion or substitution of not more than 1 amino acids.

In some embodiments, the first antigen-binding moiety comprises:

a) a heavy chain CDR1 comprising an amino acid sequence represented by SEQ ID NO: 1,
b) a heavy chain CDR2 comprising an amino acid sequence represented by SEQ ID NO: 2,
c) a heavy chain CDR3 comprising an amino acid sequence represented by SEQ ID NO: 3,
d) a light chain CDR1 comprising an amino acid sequence represented by SEQ ID NO: 4,
e) a light chain CDR2 comprising an amino acid sequence represented by SEQ ID NO: 5, and
f) a light chain CDR3 comprising an amino acid sequence represented by SEQ ID NO: 6.

In some embodiments, the first antigen-binding moiety comprises:

a) a heavy chain CDR1 consisting of an amino acid sequence represented by SEQ ID NO: 1,
b) a heavy chain CDR2 consisting of an amino acid sequence represented by SEQ ID NO: 2,
c) a heavy chain CDR3 consisting of an amino acid sequence represented by SEQ ID NO: 3,
d) a light chain CDR1 consisting of an amino acid sequence represented by SEQ ID NO: 4,
e) a light chain CDR2 consisting of an amino acid sequence represented by SEQ ID NO: 5, and
f) a light chain CDR3 consisting of an amino acid sequence represented by SEQ ID NO: 6.

In some embodiments, the heavy chain variable domain (VH1) of the first antigen-binding moiety comprises:

(i) an amino acid sequence of SEQ ID NO: 13, and

(ii) an amino acid sequence at least 85%, for example, 86%, 87%, 88%, 89%, 90%, 91%, 92%, 93%, 94%, 95%, 96%, 97%, 98%, or 99% identical to SEQ ID NO: 13 and at the same time maintaining the binding specificity to CD3; or

(iii) an amino acid sequence with addition, deletion and/or substitution of one or more amino acids (e.g., 1 to 18, 1 to 15, 1 to 10, or 1 to 5) compared with SEQ ID NO: 13 and at the same time maintaining the binding specificity to CD3.

In some embodiments, the light chain variable domain (VL1) of the first antigen-binding moiety comprises:

(i) an amino acid sequence of SEQ ID NO: 14, and

(ii) an amino acid sequence at least 85%, for example, 86%, 87%, 88%, 89%, 90%, 91%, 92%, 93%, 94%, 95%, 96%, 97%, 98%, or 99% identical to SEQ ID NO: 14 and at the same time maintaining the binding specificity to CD3; or

(iii) an amino acid sequence with addition, deletion and/or substitution of one or more amino acids (e.g., 1 to 17, 1 to 15, 1 to 10, or 1 to 5) compared with SEQ ID NO: 14 and at the same time maintaining the binding specificity to CD3.

In some embodiments, the heavy chain variable domain (VH1) of the first antigen-binding moiety consists of an amino acid sequence of SEQ ID NO: 13, and the light chain variable domain (VL1) of the first antigen-binding moiety consists of an amino acid sequence of SEQ ID NO: 14.

In some embodiments, the first antigen-binding moiety comprises two polypeptide chains:

i) a first heavy chain represented by VH1-CH1-Hinge1-CH2-CH3; and

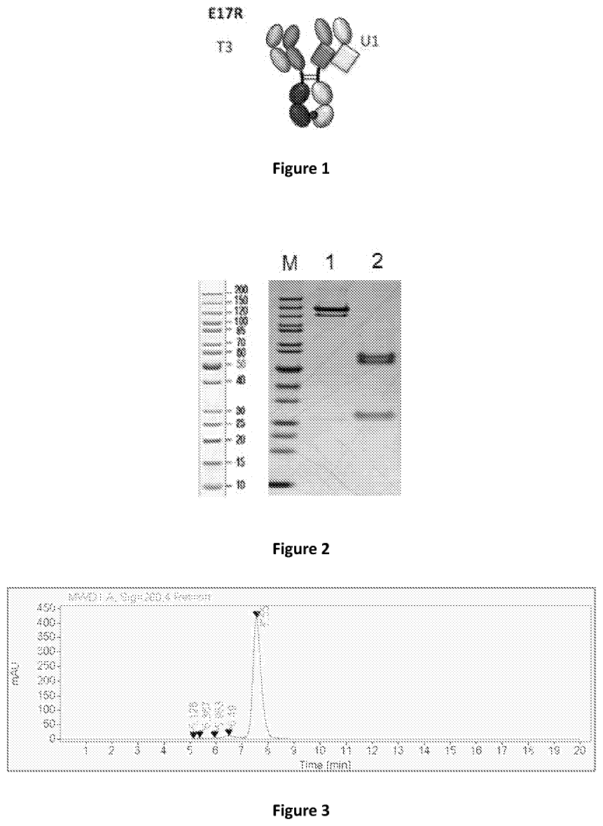

ii) a first light chain represented by VL1-CL;

wherein VH1-CH1 portion of i) and VL1-CL form an anti-CD3 arm (referred to as T3, see FIG. 1).

In some embodiments, the first antigen-binding moiety comprises two polypeptide chains:

i) a first heavy chain represented by SEQ ID NO: 23 or an amino acid sequence having at least 85%, for example, 86%, 87%, 88%, 89%, 90%, 91%, 92%, 93%, 94%, 95%, 96%, 97%, 98%, 99%, or even 100% sequence identity to SEQ ID NO: 23 and at the same time maintaining the binding specificity to CD3; and

ii) a first light chain represented by SEQ ID NO: 22 or an amino acid sequence having at least 85%, for example, 86%, 87%, 88%, 89%, 90%, 91%, 92%, 93%, 94%, 95%, 96%, 97%, 98%, 99%, or even 100% sequence identity to SEQ ID NO: 22 and at the same time maintaining the binding specificity to CD3.

In some embodiments, the first antigen-binding moiety comprises two polypeptide chains:

i) a first heavy chain represented by SEQ ID NO: 23; and

ii) a first light chain represented by SEQ ID NO: 22.

In some embodiments, the first antigen-binding moiety consists of two polypeptide chains:

i) a first heavy chain represented by SEQ ID NO: 23; and

ii) a first light chain represented by SEQ ID NO: 22.

In some embodiments, the first antigen-binding moiety is operably linked to an Fc region. Preferably, Fc region is operably linked to the CH1 domain of the CD3 antigen binding moiety.

In some embodiments, the Fc region is a human Fc region, such as a human IgG Fc region, especially a human IgG4 or IgG1 Fc region. Preferably, the Fc region is human IgG4 Fc region containing mutations S228P, F234A and L235A.

The Second Antigen-Binding Moiety that Specifically Binds to EGFR

The second antigen-binding moiety provided herein specifically binds to EGFR, and thus, it is also referred to as the EGFR antigen binding moiety in the disclosure. The two terms can be used interchangeably.

The second antigen-binding moiety comprises a chimeric Fab comprising a second heavy chain variable domain (VH2) of an anti-EGFR antibody operably linked to a first T cell receptor (TCR) constant region (C1), and a second light chain variable domain (VL2) of the anti-EGFR antibody operably linked to a second TCR constant region (C2), and wherein C1 and C2 are capable of forming a dimer via a non-native interchain disulphide bond which is capable of stabilizing the dimer.

In some embodiments, the second antigen-binding moiety comprises:

a heavy chain CDR1 comprising or consisting of an amino acid sequence selected from the group consisting of SEQ ID NO: 7 and an amino acid sequence different from SEQ ID NO: 7 by an amino acid addition, deletion or substitution of not more than 2 amino acids,

a heavy chain CDR2 comprising or consisting of an amino acid sequence selected from the group consisting of SEQ ID NO: 8 and an amino acid sequence different from SEQ ID NO: 8 by an amino acid addition, deletion or substitution of not more than 2 amino acids,

a heavy chain CDR3 comprising or consisting of an amino acid sequence selected from the group consisting of SEQ ID NO: 9 and an amino acid sequence different from SEQ ID NO: 9 by an amino acid addition, deletion or substitution of not more than 1 amino acids,

a light chain CDR1 comprising or consisting of an amino acid sequence selected from the group consisting of SEQ ID NO: 10 and an amino acid sequence different from SEQ ID NO: 10 by an amino acid addition, deletion or substitution of not more than 2 amino acids,

a light chain CDR2 comprising or consisting of an amino acid sequence selected from the group consisting of SEQ ID NO: 11 and an amino acid sequence different from SEQ ID NO: 11 by an amino acid addition, deletion or substitution of not more than 1 amino acids, and

a light chain CDR3 comprising or consisting of an amino acid sequence selected from the group consisting of SEQ ID NO: 12 and an amino acid sequence different from SEQ ID NO: 12 by an amino acid addition, deletion or substitution of not more than 1 amino acids.

In some embodiments, the second antigen-binding moiety comprises:

a) a heavy chain CDR1 comprising an amino acid sequence represented by SEQ ID NO: 7,
b) a heavy chain CDR2 comprising an amino acid sequence represented by SEQ ID NO: 8,
c) a heavy chain CDR3 comprising an amino acid sequence represented by SEQ ID NO: 9,
d) a light chain CDR1 comprising an amino acid sequence represented by SEQ ID NO: 10,
e) a light chain CDR2 comprising an amino acid sequence represented by SEQ ID NO: 11, and
f) a light chain CDR3 comprising an amino acid sequence represented by SEQ ID NO: 12.

In some embodiments, the second antigen-binding moiety comprises:

a) a heavy chain CDR1 consisting of an amino acid sequence represented by SEQ ID NO: 7,
b) a heavy chain CDR2 consisting of an amino acid sequence represented by SEQ ID NO: 8,
c) a heavy chain CDR3 consisting of an amino acid sequence represented by SEQ ID NO: 9,
d) a light chain CDR1 consisting of an amino acid sequence represented by SEQ ID NO: 10,
e) a light chain CDR2 consisting of an amino acid sequence represented by SEQ ID NO: 11, and
f) a light chain CDR3 consisting of an amino acid sequence represented by SEQ ID NO: 12.

In some embodiments, the heavy chain variable domain (VH2) of the second antigen-binding moiety comprises:

(i) an amino acid sequence of SEQ ID NO: 15, and

(ii) an amino acid sequence at least 85%, for example, 86%, 87%, 88%, 89%, 90%, 91%, 92%, 93%, 94%, 95%, 96%, 97%, 98%, or 99% identical to SEQ ID NO: 15 and at the same time maintaining the binding specificity to EGFR; or

(iii) an amino acid sequence with addition, deletion and/or substitution of one or more amino acids (e.g., 1 to 18, 1 to 15, 1 to 10, or 1 to 5) compared with SEQ ID NO: 15 and at the same time maintaining the binding specificity to EGFR.

In some embodiments, the light chain variable domain (VL2) of the second antigen-binding moiety comprises:

(i) an amino acid sequence of SEQ ID NO: 16, and

(ii) an amino acid sequence at least 85%, for example, 86%, 87%, 88%, 89%, 90%, 91%, 92%, 93%, 94%, 95%, 96%, 97%, 98%, or 99% identical to SEQ ID NO: 16 and at the same time maintaining the binding specificity to EGFR; or

(iii) an amino acid sequence with addition, deletion and/or substitution of one or more amino acids (e.g., 1 to 16, 1 to 15, 1 to 10, or 1 to 5) compared with SEQ ID NO: 16 and at the same time maintaining the binding specificity to EGFR.

In some embodiments, the heavy chain variable domain (VH2) of the second antigen-binding moiety consists of an amino acid sequence of SEQ ID NO: 15, and the light chain variable domain (VL2) of the second antigen-binding moiety consists of an amino acid sequence of SEQ ID NO: 16.

In some embodiments, the second antigen-binding moiety comprises two polypeptide chains:

i) a second heavy chain represented by VH2-C1-Hinge2-CH2-CH3, and

ii) a second light chain represented by VL2-C2,

wherein VH2-C1 of iii) and VL2-C2 form an anti-EGFR arm (referred to as U1, see FIG. 1),

wherein C1 and C2 are capable of forming a dimer comprising at least one non-native inter-chain bond, and the two hinge regions and/or the two CH3 domains are capable of forming one or more inter-chains bond that can facilitate dimerization.

In some embodiments, the second antigen-binding moiety comprises two polypeptide chains:

i) a second heavy chain represented by SEQ ID NO: 24 or an amino acid sequence having at least 85%, for example, 86%, 87%, 88%, 89%, 90%, 91%, 92%, 93%, 94%, 95%, 96%, 97%, 98%, 99%, or even 100% sequence identity to SEQ ID NO: 24 and at the same time maintaining the binding specificity to EGFR; and

ii) a second light chain represented by SEQ ID NO: 21 or an amino acid sequence having at least 85%, for example, 86%, 87%, 88%, 89%, 90%, 91%, 92%, 93%, 94%, 95%, 96%, 97%, 98%, 99%, or even 100% sequence identity to SEQ ID NO: 21 and at the same time maintaining the binding specificity to EGFR.

In some embodiments, the second antigen-binding moiety comprises two polypeptide chains:

i) a second heavy chain represented by SEQ ID NO: 24; and

ii) a second light chain represented by SEQ ID NO: 21.

In some embodiments, the second antigen-binding moiety consists of two polypeptide chains:

i) a second heavy chain represented by SEQ ID NO: 24; and

ii) a second light chain represented by SEQ ID NO: 21.

In some embodiments, the first T cell receptor (TCR) constant region (C1 domain) comprises a TCRβ constant region comprising the amino acid sequence of SEQ ID NO: 29, and in one preferred embodiment, the C1 domain comprises or consists of a TCRβ constant region represented by SEQ ID NO: 29.

In some embodiments, the second T cell receptor (TCR) constant region (C2 domain) comprises a TCRα constant region comprising the amino acid sequence of SEQ ID NO: 30, and in one preferred embodiment, the C2 domain comprises or consists of a TCRα constant region represented by SEQ ID NO: 30.

In some embodiments, the C1 domain comprises the amino acid sequence of SEQ ID NO: 29, and the C2 domain comprises the amino acid sequence of SEQ ID NO: 30.

The assignment of amino acids to each CDR or to each VH or VL may be in accordance with one of the numbering schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co. unless otherwise noted.

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinforg.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

In other embodiments, the amino acid sequences of CDRs can be at least 90%, 91%, 92%, 93%, 94%, 95%, 96%, 97%, 98% or 99% identical to the respective sequences set forth above. In other embodiments, the amino acid sequences of the variable region can be at least 90%, 91%, 92%, 93%, 94%, 95%, 96%, 97%, 98% or 99% identical to the respective sequences set forth above.

Preferably, the CDRs of the isolated antibody or the antigen-binding portion thereof contain a conservative substitution of not more than 2 amino acids, or not more than 1 amino acid. The term “conservative substitution”, as used herein, refers to amino acid substitutions which would not disadvantageously affect or change the essential properties of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, having similar size, shape, charge, chemical property including the capability of forming covalent bond or hydrogen bond, etc.) to the corresponding amino acid residue. The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having β-branched side chains (such as threonine, valine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem. 32: 1180-1187 (1993); Kobayashi et al., Protein Eng. 12(10): 879-884 (1999); and Burks et al., Proc. Natl. Acad. Sci. USA 94: 412-417 (1997), which are incorporated herein by reference).

In certain embodiments, the first antigen-binding moiety and the second antigen-binding moiety of the bispecific antibody may be connected to one another by a linker.

In certain embodiments, the bispecific antibody further comprises a Fc region that is operably linked to the first antigen-binding moiety or the second antigen-binding moiety. In some embodiments, the Fc region is operably linked to the CH1 domain of the CD3 antigen binding moiety.

The Fc region of the bispecific antibodies of the present disclosure may be human Fc region. The Fc region of the bispecific antibodies of the present disclosure may be of any isotype, including, but not limited to, IgG1, IgG2, IgG3 or IgG4. In one embodiment of this method, the Fc region is of the IgG4 isotype.

In the context of bispecific antibodies of the present disclosure, the Fc region may comprise one or more amino acid changes (e.g., insertions, deletions or substitutions) as compared to the specified chimeric version of the Fc region, without changing the desired functionality. For example, the disclosure includes bispecific antigen-binding molecules comprising one or more modifications in the Fc region that results in a modified Fc region having a modified binding interaction (e.g., enhanced or diminished) between Fe and FcRn. Non-limiting examples of such Fc modifications include, e.g., a mutation of serine (“S”) to proline (“P”) at position 228 of the amino acid sequence of human IgG4 Fc region.

In certain embodiments, the first and/or the second antigen binding moiety is bivalent. The terms “bivalent” denotes the presence of two binding site respectively, in an antigen-binding molecule. This, in certain embodiments, provides for stronger binding to the antigen or the epitope than a monovalent counterpart. In certain embodiments, in a bivalent antigen-binding moiety, the first valent of binding site and the second valent of binding site are structurally identical (i.e. having the same sequences).

TCR Constant Region

Human TCR alpha chain constant region is known as TRAC, with the NCBI accession number of P01848 (https://www.uniprot.org/uniprot/P01848), the sequence of WT TCR alpha domain is:

IQNPDPAVYQLRDSKSSDKSVCLFTDFDSQTNVSQSKDSDVYITDKTVLD
MRSMDFKSNSAVAWSNKSDFACANAFNNSIIPEDTFFPSPESS.

The engineered TCR alpha constant domain in the invention is:

(SEQ ID NO: 30)
PDIQNPDPAVYQLRDSKSSDKSVCLFTDFDSQTQVSQSKDSDVYITDKCV
LDMRSMDFKSNSAVAWSQKSDFACANAFQNSIIPEDTFFPSPESS.

Human TCR beta chain constant region has two different variants, known as TRBC1 and TRBC2 (IMGT nomenclature). In the invention, the sequence of wild type TCR beta domain is: DLKNVFPPKVAVFEPSEAEISHTQKATLVCLATGFYPDHVELSWWVNGKEVHSGVSTD PQPLKEQPALNDSRYCLSSRLRVSATFWQNPRNHFRCQVQFYGLSENDEWTQDRAKPV TQIVSAEAWGR, with the NCBI accession number of A0A5B9 (https://www.uniprot.org/uniprot/A0A5B9) and the engineered TCR beta constant domain in the invention is:

(SEQ ID NO: 29)
LEDLKNVFPPEVAVFEPSEAEISHTQKATLVCLATGFYPDHVELSWWVNG
KEVHSGVCTDPQPLKEQPALQDSRYALSSRLRVSATFWQNPRNHFRCQVQ
FYGLSENDEWTQDRAKPVTQIVSAEAWGR.

In the present disclosure, the first and the second TCR constant regions of the polypeptide complexes provided herein are capable of forming a dimer comprising, between the TCR constant regions, at least one non-native interchain bond that is capable of stabilizing the dimer.

The term “dimer” as used herein refers to an associated structure formed by two molecules, such as polypeptides or proteins, via covalent or non-covalent interactions. A homodimer or homodimerization is formed by two identical molecules, and a heterodimer or heterodimerization is formed by two different molecules. The dimer formed by the first and the second TCR constant regions is a heterodimer.

An interchain bond is formed between one amino acid residue on one TCR constant region and another amino acid residue on the other TCR constant region. In certain embodiments, the non-native interchain bond can be any bond or interaction that is capable of associating two TCR constant regions into a dimer. Examples of suitable non-native interchain bond include, a disulphide bond, a hydrogen bond, electrostatic interaction, a salt bridge, or hydrophobic-hydrophilic interaction, a knobs-into-holes or the combination thereof.

A “disulphide bond” refers to a covalent bond with the structure R—S—S—R′. The amino acid cysteine comprises a thiol group that can form a disulphide bond with a second thiol group, for example from another cysteine residue. The disulphide bond can be formed between the thiol groups of two cysteine residues residing respectively on the two polypeptide chains, thereby forming an interchain bridge or interchain bond.

A “non-native” interchain bond as used herein refers to an interchain bond which is not found in a native association of the native counterpart TCR constant regions. For example, a non-native interchain bond can be formed between a mutated amino acid residue and a native amino acid residue, each residing on a respective TCR constant region; or alternatively between two mutated amino acid residues residing respectively on the TCR constant regions. In certain embodiments, the at least one non-native interchain bond is formed between a first mutated residue comprised in the first TCR constant region and a second mutated residue comprised in the second TCR constant region of the polypeptide complex.

The term “contact interface” as used herein refers to the particular region (s) on the polypeptides where the polypeptides interact/associate with each other. A contact interface comprises one or more amino acid residues that are capable of interacting with the corresponding amino acid residue (s) that comes into contact or association when interaction occurs. The amino acid residues in a contact interface may or may not be in a consecutive sequence. For example, when the interface is three-dimensional, the amino acid residues within the interface may be separated at different positions on the linear sequence.

Generation of Bispecific Antibodies

The bispecific antibodies and antigen-binding fragments provided herein can be made with any suitable methods known in the art. In a conventional approach, two immunoglobulin heavy chain-light chain pairs can be co-expressed in a host cell to produce bispecific antibodies in a recombinant way (see, for example, Milstein and Cuello, Nature, 305: 537 (1983)), followed by purification by affinity chromatography.

Recombinant approach may also be used, where sequences encoding the antibody heavy chain variable domains for the two specificities are respectively fused to immunoglobulin constant domain sequences, followed by insertion to an expression vector which is co-transfected with an expression vector for the light chain sequences to a suitable host cell for recombinant expression of the bispecific antibody (see, for example, WO 94/04690; Suresh et al., Methods in Enzymology, 121:210 (1986)). Similarly, scFv dimers can also be recombinantly constructed and expressed from a host cell (see, e.g. Gruber et al., J. Immunol., 152:5368 (1994).) In another method, leucine zipper peptides from the Fos and Jun proteins can be linked to the Fab′ portions of two different antibodies by gene fusion. The linked antibodies are reduced at the hinge region to four half antibodies (i.e. monomers) and then re-oxidized to form heterodimers (Kostelny et al., J. Immunol., 148(5):1547-1553 (1992)).

The two antigen-binding domains may also be conjugated or cross-linked to form a bispecific antibody or antigen-binding fragment. For example, one antibody can be coupled to biotin while the other antibody to avidin, and the strong association between biotin and avidin would complex the two antibodies together to form a bispecific antibody (see, for example, U.S. Pat. No. 4,676,980 B2; WO 91/00360, WO 92/00373, and EP 03089). For another example, the two antibodies or antigen-binding fragments can be cross-linked by conventional methods known in the art, for example, as disclosed in U.S. Pat. No. 4,676,980 B2.

Bispecific antigen-binding fragments may be generated from a bispecific antibody, for example, by proteolytic cleavage, or by chemical linking. For example, an antigen-binding fragment (e.g. Fab⁵) of an antibody may be prepared and converted to Fab′-thiol derivative and then mixed and reacted with another converted Fab⁵ derivative having a different antigenic specificity to form a bispecific antigen-binding fragment (see, for example, Brennan et al., Science, 229: 81 (1985)).

Nucleic Acid Molecules Encoding the Antibody of the Disclosure

In some aspects, the disclosure is directed to an isolated nucleic acid molecule, comprising a nucleic acid sequence encoding the bispecific antibody or the antigen-binding portion as disclosed herein. For example, the nucleic acid sequence may encode the heavy chain and/or the light chain of the bispecific antibody.

An isolated nucleic acid molecule encoding the heavy chain variable domain (VH1) of the CD3 binding moiety may comprise a nucleic acid sequence selected from the group consisting of:

(A) a nucleic acid sequence that encodes the heavy chain variable domain (VH1) as set forth in SEQ ID NO: 13;

(B) a nucleic acid sequence as set forth in SEQ ID NO: 17; or

(C) a nucleic acid sequence that hybridized under high stringency conditions to the complementary strand of the nucleic acid sequence of (A) or (B).

An isolated nucleic acid molecule encoding the light chain variable domain (VL1) of the CD3 binding moiety may comprise a nucleic acid sequence selected from the group consisting of:

(A) a nucleic acid sequence that encodes the light chain variable domain (VL1) as set forth in SEQ ID NO: 14;

(B) a nucleic acid sequence as set forth in SEQ ID NO: 18; or

(C) a nucleic acid sequence that hybridized under high stringency conditions to the complementary strand of the nucleic acid sequence of (A) or (B).

An isolated nucleic acid molecule encoding the heavy chain variable domain (VH2) of the EGFR binding moiety may comprise a nucleic acid sequence selected from the group consisting of:

(A) a nucleic acid sequence that encodes the heavy chain variable domain (VH1) as set forth in SEQ ID NO: 15;

(B) a nucleic acid sequence as set forth in SEQ ID NO: 19; or

(C) a nucleic acid sequence that hybridized under high stringency conditions to the complementary strand of the nucleic acid sequence of (A) or (B).

An isolated nucleic acid molecule encoding the light chain variable domain (VL2) of the EGFR binding moiety may comprise a nucleic acid sequence selected from the group consisting of:

(A) a nucleic acid sequence that encodes light chain variable domain (VL2) as set forth in SEQ ID NO: 16;

(B) a nucleic acid sequence as set forth in SEQ ID NO: 20; or

(C) a nucleic acid sequence that hybridized under high stringency conditions to the complementary strand of the nucleic acid sequence of (A) or (B).

In some embodiments, the present disclosure provides an isolated nucleotide sequence encoding the heavy chain of the CD3 binding moiety, wherein the isolated nucleotide sequence encoding the heavy chain of the CD3 binding moiety comprises or consists of:

(A) a nucleic acid sequence that encodes the heavy chain as set forth in SEQ ID NO: 23;

(B) a nucleic acid sequence as set forth in SEQ ID NO: 27; or

(C) a nucleic acid sequence that hybridized under high stringency conditions to the complementary strand of the nucleic acid sequence of (A) or (B).

In some embodiments, the present disclosure provides an isolated nucleotide sequence encoding the light chain of the CD3 binding moiety, wherein the isolated nucleotide sequence encoding the light chain of the CD3 binding moiety comprises or consists of:

(A) a nucleic acid sequence that encodes the heavy chain as set forth in SEQ ID NO: 22;

(B) a nucleic acid sequence as set forth in SEQ ID NO: 26; or

(C) a nucleic acid sequence that hybridized under high stringency conditions to the complementary strand of the nucleic acid sequence of (A) or (B).

In some embodiments, the present disclosure provides an isolated nucleotide sequence encoding the heavy chain of the EGFR binding moiety, wherein the isolated nucleotide sequence encoding the heavy chain of the EGFR binding moiety comprises or consists of:

(A) a nucleic acid sequence that encodes the heavy chain as set forth in SEQ ID NO: 24;

(B) a nucleic acid sequence as set forth in SEQ ID NO: 28; or

(C) a nucleic acid sequence that hybridized under high stringency conditions to the complementary strand of the nucleic acid sequence of (A) or (B).

In some embodiments, the present disclosure provides an isolated nucleotide sequence encoding the light chain of the EGFR binding moiety, wherein the isolated nucleotide sequence encoding the light chain of the EGFR binding moiety comprises or consists of:

(A) a nucleic acid sequence that encodes the heavy chain as set forth in SEQ ID NO: 21;

(B) a nucleic acid sequence as set forth in SEQ ID NO: 25; or

(C) a nucleic acid sequence that hybridized under high stringency conditions to the complementary strand of the nucleic acid sequence of (A) or (B).

In some aspects, the disclosure is directed to a vector comprising the nucleic acid sequence as disclosed herein. In a further embodiment, the expression vector further comprises a nucleotide sequence encoding the constant region of a bispecific antibody, e.g. a humanized bispecific antibody.

A vector in the context of the present disclosure may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one embodiment, a CD3 or a EGFR antibody-encoding nucleic acid is comprised in a naked DNA or RNA vector, including, for example, a linear expression element (as described in for instance Sykes and Johnston, Nat Biotech 17, 355-59 (1997)), a compacted nucleic acid vector (as described in for instance U.S. Pat. No. 6,077,835 and/or WO 00/70087), a plasmid vector such as pBR322, pUC 19/18, or pUC 118/119, a “midge” minimally-sized nucleic acid vector (as described in for instance Schakowski et al., Mol Ther 3, 793-800 (2001)), or as a precipitated nucleic acid vector construct, such as a CaP04-precipitated construct (as described in for instance WO200046147, Benvenisty and Reshef, PNAS USA 83, 9551-55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In one embodiment, the vector is suitable for expression of the anti-CD3 antibody and/or anti-EGFR antibody in a bacterial cell. Examples of such vectors include expression vectors such as BlueScript (Stratagene), pIN vectors (Van Heeke & Schuster, J Biol Chem 264, 5503-5509 (1989), pET vectors (Novagen, Madison Wis.) and the like). A vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH (reviewed in: F. Ausubel et al., ed. Current Protocols in Molecular Biology, Greene Publishing and Wiley InterScience New York (1987), and Grant et al., Methods in Enzymol 153, 516-544 (1987)).

A vector may also or alternatively be a vector suitable for expression in mammalian cells, e.g. a vector comprising glutamine synthetase as a selectable marker, such as the vectors described in Bebbington (1992) Biotechnology (NY) 10: 169-175.

A nucleic acid and/or vector may also comprise a nucleic acid sequence encoding a secretion/localization sequence, which can target a polypeptide, such as a nascent polypeptide chain, to the periplasmic space or into cell culture media. Such sequences are known in the art, and include secretion leader or signal peptides.

The vector may comprise or be associated with any suitable promoter, enhancer, and other expression-facilitating elements. Examples of such elements include strong expression promoters (e. g., human CMV IE promoter/enhancer as well as RSV, SV40, SL3-3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE.

In an even further aspect, the disclosure relates to a host cell comprising the vector specified herein above.

Thus, the present disclosure also relates to a recombinant eukaryotic or prokaryotic host cell which produces a bispecific antibody of the present disclosure, such as a transfectoma.

The CD3-specific antibody may be expressed in a recombinant eukaryotic or prokaryotic host cell, such as a transfectoma, which produces an antibody of the disclosure as defined herein or a bispecific antibody of the disclosure as defined herein. The EGFR-specific antibody may likewise be expressed in a recombinant eukaryotic or prokaryotic host cell, such as a transfectoma, which produces an antibody of the disclosure as defined herein or a bispecific antibody of the disclosure as defined herein.

Examples of host cells include yeast, bacterial, plant and mammalian cells, such as CHO, CHO-S, HEK, HEK293, HEK-293F, Expi293F, PER.C6 or NSO cells or lymphocytic cells. For example, in one embodiment, the host cell may comprise a first and second nucleic acid construct stably integrated into the cellular genome. In another embodiment, the present disclosure provides a cell comprising a non-integrated nucleic acid, such as a plasmid, cosmid, phagemid, or linear expression element, which comprises a first and second nucleic acid construct as specified above.

In an even further aspect, the disclosure relates to a transgenic non-human animal or plant comprising nucleic acids encoding one or two sets of a human heavy chain and a human light chain, wherein the animal or plant produces a bispecific antibody of the disclosure.

In a further aspect, the disclosure relates to a hybridoma which produces an antibody for use in a bispecific antibody of the disclosure as defined herein. In an even further aspect, the disclosure relates to a transgenic non-human animal or plant comprising nucleic acids encoding one or two sets of a human heavy chain and a human light chain, wherein the animal or plant produces an antibody for use in a bispecific antibody or a bispecific antibody of the disclosure.

In one aspect, the disclosure relates to an expression vector comprising:

(i) a nucleic acid sequence encoding a heavy chain variable region of the first antigen-binding moiety and/or a heavy chain variable region of the second antigen-binding moiety according to any one of the embodiments disclosed herein, optionally, further encoding the CH1 domain or CL domain;

(ii) a nucleic acid sequence encoding a light chain variable region of the first antigen-binding moiety and/or a light chain variable region of the second antigen-binding moiety according to any one of the embodiments disclosed herein;

(iii) a nucleic acid sequence encoding the TCR beta constant domain or the TCR alpha constant domain;

(iv) a nucleic acid sequence encoding a Fc region;

(v) a nucleic acid sequence encoding a linker; or

(vi) the combinations of at least two of the above.

In one aspect, the disclosure relates to a nucleic acid construct encoding one or more amino acid sequences set forth in the sequence listing.

In one aspect, the disclosure relates to a method for producing a bispecific antibody according to any one of the embodiments as disclosed herein, comprising the steps of culturing a host cell as disclosed herein comprising an expression vector or more than one expression vectors as disclosed herein expressing the bispecific antibody as disclosed herein and purifying said antibody from the culture media. In one aspect, the disclosure relates to a host cell comprising an expression vector as defined above. In one embodiment, the host cell is a recombinant eukaryotic, recombinant prokaryotic, or recombinant microbial host cell.

Pharmaceutical Compositions

In some aspects, the disclosure is directed to a pharmaceutical composition comprising at least one antibody or antigen-binding portion thereof as disclosed herein and a pharmaceutically acceptable carrier.

Components of the Compositions

The pharmaceutical composition may optionally contain one or more additional pharmaceutically active ingredients, such as another antibody or a drug. The pharmaceutical compositions of the disclosure also can be administered in a combination therapy with, for example, another immune-stimulatory agent, anti-cancer agent, an antiviral agent, or a vaccine, such that the anti-CD3/anti-EGFR bispecific antibody enhances the immune response against the vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed in the present disclosure, in a solvent containing an antibody or an antigen-binding fragment of the present disclosure discloses compositions include one or more anti-oxidants such as methionine, reducing antibody or antigen binding fragment thereof may be oxidized. The oxidation reduction may prevent or reduce a decrease in binding affinity, thereby enhancing antibody stability and extended shelf life. Thus, in some embodiments, the present disclosure provides a composition comprising one or more antibodies or antigen binding fragment thereof and one or more anti-oxidants such as methionine. The present disclosure further provides a variety of methods, wherein an antibody or antigen binding fragment thereof is mixed with one or more anti-oxidants, such as methionine, so that the antibody or antigen binding fragment thereof can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethyl celluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetra-acetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the antibody or the antigen binding portion thereof of the disclosure may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight per dose.

In any event, the antibody or the antigen binding portion thereof of the disclosure is preferably administered as needed to a subject in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the antibody or the antigen-binding portion thereof of the instant disclosure will comprise multiple doses of the selected drug product over a period of weeks or months. More specifically, the antibody or the antigen-binding portion thereof of the instant disclosure may be administered once every day, every two days, every four days, every week, every ten days, every two weeks, every three weeks, every month, every six weeks, every two months, every ten weeks or every three months. In this regard, it will be appreciated that the dosages may be altered, or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the antibody or antigen-binding portion thereof as disclosed herein in concentrations of from about 10 μg/ml to about 100 mg/ml. In certain selected embodiments, the concentrations of the antibody or the antigen binding portion thereof will comprise 20 μg/ml, 40 μg/ml, 60 μg/ml, 80 μg/ml, 100 μg/ml, 200 μg/ml, 300, g/ml, 400 μg/ml, 500 μg/ml, 600 μg/ml, 700 μg/ml, 800 μg/ml, 900 μg/ml or 1 mg/ml. In other preferred embodiments ADC concentrations will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml.

Applications of the Disclosure

In some aspects, the present disclosure provides a method of treating a disorder in a subject, which comprises administering to the subject (for example, a human) in need of treatment a therapeutically effective amount of the antibody or antigen-binding portion thereof as disclosed herein. For example, the disorder is a cancer.

A variety of cancers where CD3 and/or EGFR is implicated, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; skin cancers such as basal cell carcinoma, squamous cell carcinoma, melanoma, Kaposi's sarcoma, and Paget's disease; head and neck cancers; eye-related cancers such as retinoblastoma and 58hlorambuci melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers. In a specific embodiment, the cancer is colon cancer. Combined use with chemotherapies

The antibody or the antigen-binding portion thereof may be used in combination with an anti-cancer agent, a cytotoxic agent or chemotherapeutic agent.

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in selected embodiments as discussed above, such anti-cancer agents may comprise conjugates and may be associated with the disclosed site-specific antibodies prior to administration. More specifically, in certain embodiments selected anti-cancer agents will be linked to the unpaired cysteines of the engineered antibodies to provide engineered conjugates as set forth herein. Accordingly, such engineered conjugates are expressly contemplated as being within the scope of the instant disclosure. In other embodiments, the disclosed anti-cancer agents will be given in combination with site-specific conjugates comprising a different therapeutic agent as set forth above.

As used herein the term “cytotoxic agent” means a substance that is toxic to the cells and decreases or inhibits the function of cells and/or causes destruction of cells. In certain embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, 59hlorambu officinalis inhibitor, gelonin, mitegellin, restrictocin, phenomycin, neomycin, and the tricothecenes) or animals, (e.g., cytotoxic Rnases, such as extracellular pancreatic Rnases; Dnase I, including fragments and/or variants thereof).

For the purposes of the instant disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the site-specific constructs of the present disclosure (either as a component of a site specific conjugate or in an unconjugated state) include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Combined Use with Radiotherapies

The present disclosure also provides for the combination of the antibody or the antigen-binding portion thereof with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed conjugates may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

Pharmaceutical Packs and Kits

Pharmaceutical packs and kits comprising one or more containers, comprising one or more doses of the antibody or the antigen-binding portion thereof are also provided. In certain embodiments, a unit dosage is provided wherein the unit dosage contains a predetermined amount of a composition comprising, for example, the antibody or the antigen-binding portion thereof, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the conjugate composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed conjugate composition is used for treating the neoplastic disease condition of choice.

The present disclosure also provides kits for producing single-dose or multi-dose administration units of site-specific conjugates and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed conjugates in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the engineered conjugate and, optionally, one or more anti-cancer agents in the same or different containers. The kits may also contain other pharmaceutically acceptable formulations, either for diagnosis or combined therapy. For example, in addition to the antibody or the antigen-binding portion thereof of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed the antibody or the antigen-binding portion thereof, with or without additional components, or they may have distinct containers for each desired agent. Where combined therapeutics are provided for conjugation, a single solution may be pre-mixed, either in a molar equivalent combination, or with one component in excess of the other. Alternatively, the conjugates and any optional anti-cancer agent of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody or the antigen-binding portion thereof and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

Sequence Listing Summary

Appended to the instant application is a sequence listing comprising a number of amino acid sequences and nucleotide sequences. The following Tables A to provides a summary of the included sequences.

One illustrative antibody as disclosed herein, which is an anti-CD3/anti-EGFR bispecific antibody, is designated as “W3448-T3U1.E17R-1.uIgG4V9” and is referred to as the lead BsAb hereinafter.

TABLE A
CDR amino acid sequences

| moiety | portion | CDR1 | CDR2 | CDR3 |
|---|---|---|---|---|
| T3 (anti-CD3 arm) | HCDR | SEQ ID NO: 1 | SEQ ID NO: 2 | SEQ ID NO: 3 |
| | | GFAFTDYYIH | WISPGNVNTKYN | DGYSLYYFD |
| | | | ENFKG | Y |
| | LCDR | SEQ ID NO: 4 | SEQ ID NO: 5 | SEQ ID NO: 6 |
| | | KSSQSLLNSRTR | WASTRQS | TQSHTLRT |
| | | KNYLA | | |
| U1 (anti-EGFR arm) | HCDR | SEQ ID NO: 7 | SEQ ID NO: 8 | SEQ ID NO: 9 |
| | | GGSVSSGDYYWT | HIYYSGNTNYNP | DRVTGAFDI |
| | | | SLKS | |
| | LCDR | SEQ ID NO: 10 | SEQ ID NO: 11 | SEQ ID NO: 12 |
| | | QASQDISNYLN | DASNLET | QHFDHLPLA |

TABLE B
Amino acid sequences of variable region

| moiety | VH | VL |
|---|---|---|
| T3 | SEQ ID NO: 13 | SEQ ID NO: 14 |
| (anti-CD3 arm) | QVQLVQSGAEVKKPGSSVKVSCKASGFAFT | DIVMTQSPDSLAVSLGERATINCKSSQ |
| | DYYIHWVRQAPGQGLEWMGWISPGNVNTKY | SLLNSRTRKNYLAWYQQKPGQPPKLLI |
| | NENFKGRVTITADKSTSTAYMELSSLRSED | YWASIRQSGVPDRFSGSGSGTDFTLTI |
| | TAVYYCARDGYSLYYFDYWGQGTLVTVSS | SSLQAEDVAVYYCTQSHTLRTFGGGTK |
| | | VEIK |
| Ul | SEQ ID NO: 15 | SEQ ID NO: 16 |
| (anti-EGFR arm) | QVQLQESGPGLVKPSETLSLTCTVSGGSVS | DIQMTQSPSSLSASVGDRVTITCQASQ |
| | SGDYYWTWIRQSPGKGLEWIGHIYYSGNTN | DISNYLNWYQQKPGKAPKLLIYDASNL |
| | YNPSLKSRLTISIDTSKTQFSLKLSSVTAA | ETGVPSRFSGSGSGTDFTFTISSLQPE |
| | DTAIYYCVRDRVTGAFDIWGQGTMVTV | DIATYFCQHFDHLPLAFGGGTKVEIK |

TABLE C
Nucleotide sequences of variable region

| moiety | VH | VL |
|---|---|---|
| T3 | SEQ ID NO: 17 | SEQ ID NO: 18 |
| (anti-CD3 arm) | CAGGTGCAGCTTGTGCAGTCTGGGGCAG | GATATCGTGATGACCCAGAGCCCAGA |
| | AAGTGAAGAAGCCTGGGTCTAGTGTCAA | CTCCCTTGCTGTCTCCCTCGGCGAAA |
| | GGTGTCATGCAAGGCTAGCGGGTTCGCC | GAGCAACCATCAACTGCAAGAGCTCC |
| | TTTACTGACTACTACATCCACTGGGTGC | CAAAGCCTGCTGAACTCCAGGACCAG |
| | GGCAGGCTCCCGGACAAGGGTTGGAGTG | GAAGAATTACCTGGCCTGGTATCAGC |
| | GATGGGATGGATCTCCCCAGGCAATGTC | AGAAGCCCGGCCAGCCTCCTAAGCTG |
| | AACACAAAGTACAACGAGAACTTCAAAG | CTCATCTACTGGGCCTCCACCCGGCA |
| | GCCGCGTCACCATTACCGCCGACAAGAG | GTCTGGGGTGCCCGATCGGTTTAGTG |
| | CACCTCCACAGCCTACATGGAGCTGTCC | GATCTGGGAGCGGGACAGACTTCACA |
| | AGCCTCAGAAGCGAGGACACTGCCGTCT | TTGACAATTAGCTCACTGCAGGCCGA |
| | ACTACTGTGCCAGGGATGGGTACTCCCT | GGACGTGGCCGTCTACTACTGTACTC |
| | GTATTACTTTGATTACTGGGGCCAGGGC | AGAGCCACACTCTCCGCACATTCGGC |
| | ACACTGGTGACAGTGAGCTCC | GGAGGGACTAAAGTGGAGATTAAG |
| U1 | SEQ ID NO: 19 | SEQ ID NO: 20 |
| (anti-EGFR arm) | CAGGTGCAACTGCAGGAAAGCGGACCAG | GATATCCAGATGACCCAGTCCCCTTC |
| | GACTTGTGAAGCCCTCTGAGACTTTGTC | CTCCTTGTCCGCAAGTGTGGGAGATA |
| | CCTGACCTGTACCGTCTCCGGGGGCTCT | GAGTGACCATCACATGCCAGGCTTCT |
| | GTCAGTTCAGGGGATTACTACTGGACAT | CAGGACATCTCTAACTACCTGAACTG |
| | GGATCAGGCAGAGTCCTGGGAAAGGCCT | GTACCAACAGAAGCCCGGCAAGGCCC |
| | GGAGTGGATTGGGCACATCTACTACTCA | CTAAGCTCCTTATCTACGACGCCTCA |
| | GGGAACACCAACTACAATCCCAGCCTCA | AATCTGGAGACCGGAGTCCCAAGCAG |
| | AGAGCAGACTGACCATCAGCATTGACAC | GTTCAGCGGCAGCGGGAGCGGGACAG |
| | CTCCAAGACACAGTTCTCCCTGAAGCTC | ATTTCACTTTTACAATTAGCTCCCTC |
| | AGCAGCGTGACTGCCGCCGACACAGCAA | CAGCCAGAAGACATTGCCACATATTT |
| | TCTACTATTGCGTGCGCGACCGGGTGAC | CTGTCAGCACTTTGACCATCTGCCCC |
| | AGGCGCTTTTGATATTTGGGGCCAGGGC | TGGCCTTTGGGGGCGGGACTAAAGTG |
| | ACAATGGTCACTGTG | GAGATTAAG |

TABLE D
Full length amino acid sequences of light chain and heavy chain

| | | |
|---|---|---|
| W3448- | U1-light chain- | DIQMTQSPSSLSASVGDRVTITCQASQDISNYLNWYQQKPGKAPKLLIYDA |
| T3U1.E17R-1. | SEQ ID NO: 21 | SNLETGVPSRFSGSGSGTDFTFTISSLQPEDIATYFCQHFDHLPLAFGGGT |
| uIgG4V9 | | KVEIKPDIQNPDPAVYQLRDSKSSDKSVCLFTDFDSQTQVSQSKDSDVYIT |
| | | DKCVLDMRSMDFKSNSAVAWSQKSDFACANAFQNSIIPEDTFFPSPESS |
| | T3-light chain | DIVMTQSPDSLAVSLGERATINCKSSQSLLNSRIRKNYLAWYQQKPGQPPK |
| | SEQ ID NO: 22 | LLIYWASIRQSGVPDRFSGSGSGTDFTLTISSLQAEDVAVYYCTQSHTLRT |
| | | FGGGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEVTHQ |
| | | GLSSPVTKSFNRGEC |
| | T3-heavy chain | QVQLVQSGAEVKKPGSSVKVSCKASGFAFTDYYIHWVRQAPGQGLEWMGWI |
| | SEQ ID NO: 23 | SPGNVNTKYNENFKGRVTITADKSTSTAYMELSSLRSEDTAVYYCARDGYS |
| | | LYYFDYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCNV |
| | | DHKPSNTKVDKRVESKYGPPCPPCPAPEAAGGPSVFLFPPKPKDTLMISRT |
| | | PEVTCVVVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPCQEEM |
| | | TKNQVSLWCLVKGFYPSDIAVEWESNGQPENNYKTTPPVLDSDGSFFLYSR |
| | | LTVDKSRWQEGNVFSCSVMHEALHNHYTQKSLSLSLGK |
| | Ul-heavy chain | QVQLQESGPGLVKPSETLSLTCTVSGGSVSSGDYYWTWIRQSPGKGLEWIG |
| | SEQ ID NO: 24 | HIYYSGNTNYNPSLKSRLTISIDTSKTQFSLKLSSVTAADTAIYYCVRDRV |
| | | TGAFDIWGQGTMVTVLEDLKNVFPPEVAVFEPSEAEISHTQKATLVCLATG |
| | | FYPDHVELSWWVNGKEVHSGVCTDPQPLKEQPALQDSRYALSSRLRVSATF |
| | | WQNPRNHFRCQVQFYGLSENDEWTQDRAKPVTQIVSAEAWGRYGPPCPPCP |
| | | APEAAGGPSVFLFPPKPKDTLMISRTPEVTCVVVDVSQEDPEVQFNWYVDG |
| | | VEVHNAKTKPREEQFNSTYRVVSVLTVLHQDWLNGKEYKCKVSNKGLPSSI |
| | | EKTISKAKGQPREPQVCTLPPSQEEMTKNQVSLSCAVKGFYPSDIAVEWES |
| | | NGQPENNYKTTPPVLDSDGSFFLVSRLTVDKSRWQEGNVFSCSVMHEALHN |
| | | HYTQKSLSLSLGK |

TABLE E
Full length nucleotide sequences of light chain and heavy chain

| | | |
|---|---|---|
| W3448- | U1-light chain- | GATATCCAGATGACCCAGTCCCCTTCCTCCTTGTCCGCAAGTGTGGGAG |
| T3U1.E17R-1. | SEQ ID NO: 25 | ATAGAGTGACCATCACATGCCAGGCTTCTCAGGACATCTCTAACTACCT |
| uIgG4V9 | | GAACTGGTACCAACAGAAGCCCGGCAAGGCCCCTAAGCTCCTTATCTAC |
| | | GACGCCTCAAATCTGGAGACCGGAGTCCCAAGCAGGTTCAGCGGCAGCG |
| | | GGAGCGGGACAGATTTCACTTTTACAATTAGCTCCCTCCAGCCAGAAGA |
| | | CATTGCCACATATTTCTGTCAGCACTTTGACCATCTGCCCCTGGCCTTT |
| | | GGGGGCGGGACTAAAGTGGAGATTAAGCCCGACATCCAGAACCCCGACC |
| | | CCGCCGTGTACCAGCTGAGAGACAGCAAGAGCAGCGACAAGAGCGTGTG |
| | | CCTGTTCACCGACTTCGACAGCCAGACCCAGGTGAGCCAGAGCAAGGAC |
| | | TCCGACGTGTATATCACCGACAAGTGCGTGCTGGACATGAGGAGCATGG |
| | | ACTTCAAGAGCAACAGCGCCGTGGCCTGGAGCCAGAAGAGCGACTTCGC |
| | | CTGCGCCAACGCCTTCCAGAACAGCATCATCCCCGAGGACACCTTCTTC |
| | | CCCAGCCCCGAGAGCAGC |
| | T3-light chain | GATATCGTGATGACCCAGAGCCCAGACTCCCTTGCTGTCTCCCTCGGCG |
| | SEQ ID NO: 26 | AAAGAGCAACCATCAACTGCAAGAGCTCCCAAAGCCTGCTGAACTCCAG |
| | | GACCAGGAAGAATTACCTGGCCTGGTATCAGCAGAAGCCCGGCCAGCCT |
| | | CCTAAGCTGCTCATCTACTGGGCCTCCACCCGGCAGTCTGGGGTGCCCG |
| | | ATCGGTTTAGTGGATCTGGGAGCGGGACAGACTTCACATTGACAATTAG |
| | | CTCACTGCAGGCCGAGGACGTGGCCGTCTACTACTGTACTCAGAGCCAC |
| | | ACTCTCCGCACATTCGGCGGAGGGACTAAAGTGGAGATTAAGCGTACGG |
| | | TGGCTGCACCATCTGTCTTCATCTTCCCGCCATCTGATGAGCAGTTGAA |
| | | ATCTGGAACTGCCTCTGTTGTGTGCCTGCTGAATAACTTCTATCCCAGA |
| | | GAGGCCAAAGTACAGTGGAAGGTGGATAACGCCCTCCAATCGGGTAACT |
| | | CCCAGGAGAGTGTCACAGAGCAGGACAGCAAGGACAGCACCTACAGCCT |
| | | CAGCAGCACCCTGACGCTGAGCAAAGCAGACTACGAGAAACACAAAGTC |
| | | TACGCCTGCGAAGTCACCCATCAGGGCCTGAGCTCGCCCGTCACAAAGA |
| | | GCTTCAACAGGGGAGAGTGT |
| | T3-heavy chain | CAGGTGCAGCTTGTGCAGTCTGGGGCAGAAGTGAAGAAGCCTGGGTCTA |
| | SEQ ID NO: 27 | GTGTCAAGGTGTCATGCAAGGCTAGCGGGTTCGCCTTTACTGACTACTA |
| | | CATCCACTGGGTGCGGCAGGCTCCCGGACAAGGGTTGGAGTGGATGGGA |
| | | TGGATCTCCCCAGGCAATGTCAACACAAAGTACAACGAGAACTTCAAAG |
| | | GCCGCGTCACCATTACCGCCGACAAGAGCACCTCCACAGCCTACATGGA |
| | | GCTGTCCAGCCTCAGAAGCGAGGACACTGCCGTCTACTACTGTGCCAGG |
| | | GATGGGTACTCCCTGTATTACTTTGATTACTGGGGCCAGGGCACACTGG |
| | | TGACAGTGAGCTCCGCGTCGACCAAGGGCCCATCCGTCTTCCCCCTGGC |
| | | GCCCTGCTCCAGGAGCACCTCCGAGAGCACAGCCGCCCTGGGCTGCCTG |
| | | GTCAAGGACTACTTCCCCGAACCGGTGACGGTGTCGTGGAACTCAGGCG |
| | | CCCTGACCAGCGGCGTGCACACCTTCCCGGCTGTCCTACAGTCCTCAGG |
| | | ACTCTACTCCCTCAGCAGCGTGGTGACCGTGCCCTCCAGCAGCTTGGGC |
| | | ACGAAGACCTACACCTGCAACGTAGATCACAAGCCCAGCAACACCAAGG |
| | | TGGACAAGAGAGTTGAGTCCAAATATGGTCCCCCATGCCCACCATGCCC |
| | | AGCACCTGAGGCAGCAGGGGGACCATCAGTCTTCCTGTTCCCCCCAAAA |
| | | CCCAAGGACACTCTCATGATCTCCCGGACCCCTGAGGTCACGTGCGTGG |
| | | TGGTGGACGTGAGCCAGGAAGACCCCGAGGTCCAGTTCAACTGGTACGT |
| | | GGATGGCGTGGAGGTGCATAATGCCAAGACAAAGCCGCGGGAGGAGCAG |
| | | TTCAACAGCACGTACCGTGTGGTCAGCGTCCTCACCGTCCTGCACCAGG |
| | | ACTGGCTGAACGGCAAGGAGTACAAGTGCAAGGTCTCCAACAAAGGCCT |
| | | CCCGTCCTCCATCGAGAAAACCATCTCCAAAGCCAAAGGGCAGCCCCGA |
| | | GAGCCACAGGTGTACACCCTGCCCCCATGCCAGGAGGAGATGACCAAGA |
| | | ACCAGGTCAGCCTGTGGTGCCTGGTCAAAGGCTTCTACCCCAGCGACAT |
| | | CGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAGGC |
| | | TAACCGTGGACAAGAGCAGGTGGCAGGAGGGGAATGTCTTCTCATGCTC |
| | | CGTGATGCATGAGGCTCTGCACAACCACTACACACAGAAGAGCCTCTCC |
| | | CTGTCTCTGGGTAAA |
| | U1-heavy chain | CAGGTGCAACTGCAGGAAAGCGGACCAGGACTTGTGAAGCCCTCTGAGA |
| | SEQ ID NO: 28 | CTTTGTCCCTGACCTGTACCGTCTCCGGGGGCTCTGTCAGTTCAGGGGA |
| | | TTACTACTGGACATGGATCAGGCAGAGTCCTGGGAAAGGCCTGGAGTGG |
| | | ATTGGGCACATCTACTACTCAGGGAACACCAACTACAATCCCAGCCTCA |
| | | AGAGCAGACTGACCATCAGCATTGACACCTCCAAGACACAGTTCTCCCT |
| | | GAAGCTCAGCAGCGTGACTGCCGCCGACACAGCAATCTACTATTGCGTG |
| | | CGCGACCGGGTGACAGGCGCTTTTGATATTTGGGGCCAGGGCACAATGG |
| | | TCACTGTGCTGGAGGACCTGAAGAACGTGTTCCCTCCCGAGGTGGCCGT |
| | | GTTCGAACCCAGCGAGGCCGAGATCAGCCACACCCAGAAGGCCACCCTG |
| | | GTGTGTCTGGCCACCGGCTTCTACCCCGACCACGTGGAGCTGAGCTGGT |
| | | GGGTGAACGGCAAGGAGGTGCACAGCGGCGTGTGTACCGACCCTCAGCC |
| | | CCTGAAGGAGCAGCCCGCCCTGCAGGACAGCAGGTACGCCCTGAGCAGC |
| | | AGGCTGAGAGTGAGCGCCACCTTCTGGCAGAACCCCAGGAACCACTTCA |
| | | GGTGCCAGGTGCAGTTCTACGGCCTGAGCGAGAACGACGAGTGGACCCA |
| | | GGACAGGGCCAAGCCCGTGACCCAGATCGTGAGCGCTGAGGCCTGGGGC |
| | | AGATATGGTCCCCCATGCCCACCATGCCCAGCACCTGAGGCAGCAGGGG |
| | | GACCATCAGTCTTCCTGTTCCCCCCAAAACCCAAGGACACTCTCATGAT |
| | | CTCCCGGACCCCTGAGGTCACGTGCGTGGTGGTGGACGTGAGCCAGGAA |
| | | GACCCCGAGGTCCAGTTCAACTGGTACGTGGATGGCGTGGAGGTGCATA |
| | | ATGCCAAGACAAAGCCGCGGGAGGAGCAGTTCAACAGCACGTACCGTGT |
| | | GGTCAGCGTCCTCACCGTCCTGCACCAGGACTGGCTGAACGGCAAGGAG |
| | | TACAAGTGCAAGGTCTCCAACAAAGGCCTCCCGTCCTCCATCGAGAAAA |
| | | CCATCTCCAAAGCCAAAGGGCAGCCCCGAGAGCCACAGGTGTGCACCCT |
| | | GCCCCCATCCCAGGAGGAGATGACCAAGAACCAGGTCAGCCTGAGCTGC |
| | | GCGGTCAAAGGCTTCTACCCCAGCGACATCGCCGTGGAGTGGGAGAGCA |
| | | ATGGGCAGCCGGAGAACAACTACAAGACCACGCCTCCCGTGCTGGACTC |
| | | CGACGGCTCCTTCTTCCTCGTTAGCAGGCTAACCGTGGACAAGAGCAGG |
| | | TGGCAGGAGGGGAATGTCTTCTCATGCTCCGTGATGCATGAGGCTCTGC |
| | | ACAACCACTACACACAGAAGAGCCTCTCCCTGTCTCTGGGTAAA |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Materials, Benchmark (BMK) Antibodies and Cell Lines

Information on the commercially available materials used in the examples are provided in Table F. Unless specified otherwise, the reagents and other materials used in the experiments are commercially available.

TABLE F
Commercial Materials

| | | Catalog |
|---|---|---|
| Materials | Vendor | No. (Cat.) |
| Human CD4⁺T Cell Isolation Kit | Stemcell | 19052 |
| Human CD8⁺ T Cell Isolation Kit | Stemcell | 19053 |
| Human CD3⁺T Cell Isolation Kit | Stemcell | 17951 |
| Jurkat.2B8 (Human Acute T Cell Leukemia) | ATCC | TIB-152 |
| A431 (Human Epidermoid Carcinoma) | ATCC | CRL-1555 |
| HT-29 (Human Colorectal Adenocarcinoma) | ATCC | HTB-38 |
| MCF-7 (Human Breast Cancer) | ATCC | HTB-22 |
| HCC1419 (Human Breast Cancer) | ATCC | CRL-2326 |
| Human CD3 epsilon (CD3ε) protein (His Tag) | Sino | 10977-H08H |
| | Biological | |
| | Inc. | |

TABLE G
Abbreviations

| Abbreviation | Full Name |
|---|---|
| CD3 | Cluster of differentiation 3 |
| EGFR | Epidermal growth factor receptor |
| BsAb | Bispecific antibody |
| ECD | Extra-cellular domains |
| CHOK1-cynoPro1 | Cynomolgus monkey EGFR-expressing cell line |
| cyno | Cynomolgus monkey |
| PBMC | Peripheral Blood Mononuclear Cell |
| ADCC | Antibody-dependent cell-mediated cytotoxicity |
| CDC | Complement dependent cytotoxicity |
| DSF | Differential scanning fluorimetry |
| LDH | Lactate dehydrogenase |
| PBST | Phosphate buffered saline with 0.05% (v/v) |
| | Tween 20 |
| TMB | Tetramethylbenzidine |
| ELISA | Enzyme-linked immuno sorbent assay |
| FACS | Fluorescence-activated cell sorting |

TABLE H
Materials code

| Materials Code | Materials Name/Information |
|---|---|
| W3311-2.306.4-z1-uIgG1K | Humanized anti-CD3 antibody, |
| | prepared according to standard |
| | molecular methods, see |
| | WO/2019/057099. |
| Panitumumab | Anti-EGFR antibody from U.S. Pat. |
| | No. 6,235,883 |
| WBP336-hBMK1.IgG1 | Cetuximab, from U.S. Pat. No. |
| | 6,217,866 B1 |
| W331-hPro1.ECD.His (Sino) | Human CD3 epsilon (CD3ε) protein |
| | with His tag |
| W3448-T3U1.E17R-.uIgG4V9 | Bispecific antibody of the present |
| | disclosure |

### 1.1 Production of BMK Antibodies

In the present disclosure, the anti-EGFR monoclonal antibody (Panitumumab and Cetuximab) and the anti-CD3 monoclonal antibody (W3311-2.306.4-z1-uIgG1K, refer to WO/2019/057099 for details of the preparation) as well as the anti-CD3 and anti-EGFR bispecific antibody and some BMK control antibodies were prepared according to standard molecular methods.

DNA sequences encoding the variable regions of anti-CD3 antibody, Panitumumab and Cetuximab were synthesized and cloned into expression vectors with the constant region of humanIgG1, IgG2 and IgG1, respectively.

Generally, the recombinant plasmids were transfected into EXpi293 cells according to manufacturer's instructions (Expi293F Transfection Kit, Invitrogen). The cells were cultured in an incubator at 37° C., 8% CO₂ and then the supernatant were collected after 5 days of culturing. The proteins were purified using Protein A column and SEC column.

### 1.2 Generation of Target-Expressing Cell Lines

The full-length coding gene of cynomolgus monkey EGFR was cloned into an expression vector for development of cyno EGFR-expressing cell line. Briefly, the CHO-K1 cells at 70-90% confluents were transfected with a recombinant plasmid containing the full-length coding gene of cyno EGFR by using lipofectamine 2000 reagent. The transfected cells were cultured in an incubator at 37° C., 5% CO2. 24 hours after transfection, blasticidin at a final concentration of 2-g/mL was used to select the stable pool. Then, the positive pool cells were subcloned by limited dilution. Single clone was picked and tested via FACS by using anti-EGFR antibody. The resultant cyno EGFR-expressing cell line was designated as CHOK1-cynoPro1 (EGFR+/CD3−) cell line.

Additionally, the following cell lines cultured in complete medium (RPMI 1640 supplemented with 10% FBS, 100 U/ml penicillin, and 100 μg/ml streptomycin) were used: Jurkat.2B8 (CD3+/EGFR−) cells; MCF-7 (CD3−/EGFRLow) cells and HCC1419 (CD3−/EGFR−) cells.

Other cell lines cultured in corresponding medium were used: A431 (CD3−/EGFRHigh) in DMEM supplemented with 10% FBS; HT29 (CD3−/EGFRMed) cells in MCCoy's 5A supplemented with 10% FBS.

Human and cyno peripheral blood mononuclear cells (PBMC) were freshly isolated by Ficoll-Paque PLUS (GE Healthcare-17-1440-03) density centrifugation from heparinized venous blood collected from healthy normal donors. The primary human CD8+ T cells and human CD4+ T cells were isolated from fresh human PBMC by EasySep kit (Stemcell-19053) and EasySep kit (Stemcell-19052), respectively, while human CD3+ T cells were isolated by EasySep kit (Stemcell-17951). Cyno T cells were isolated by Pan T Cell Isolation Kit_non-human primate (Miltenyi-130-091-993).

### Example 2

### Generation of Bispecific Antibody

### 2.1 Generate Bispecific Antibodies to Select Antibody Pairs

For generation of bispecific antibody, the sequence of anti-CD3 antibody (W3311-2.306.4-z1-uIgGK, made in Example 1) and the sequence of anti-EGFR antibody (Panitumumab) were used for constructing different BsAb formats.

### Results:

The pairs of antibodies for generating the bispecific antibody are identified. The results were shown in Table 1.

TABLE 1
Sequence information of antibody for preparing the bispecific antibody

| Target | | T/U |
|---|---|---|
| name | Antibody | format |
| CD3 | W3311-2.306.4-z1-uIgG1K (Humanized anti-CD3 | T3 |
| | mAb) | |
| EGFR | Panitumumab (anti-EGFR mAb) | U1 |

### 2.2 Generate Different Formats of Bispecific Antibodies

In the polypeptide complex provided herein, the first antigen-binding moiety and the second binding moiety are associated into an Ig-like structure. An Ig-like structure is like a natural antibody having a Y shaped construct, with two arms for antigen-binding and one stem for association and stabilization. The resemblance to natural antibody can provide for various advantages such as good in vivo pharmakinetics, desired immunological response and stability etc. It has been found that the Ig-like structure comprising the first antigen-binding moiety provided herein associated with the second antigen-binding moiety provided herein has thermal stability which is comparable to that of an Ig (e.g. an IgG). In certain embodiments, the Ig-like structure provided herein is at least 70%, 80%, 90%, 95% or 100% of that of a natural IgG.

The bispecific polypeptide complex provided herein comprises four polypeptide chains: i) a first heavy chain represented by VH1-CH1-Hinge1-CH2-CH3; ii) a first light chain represented by VL1-CL; iii) a second heavy chain represented by VH2-C1-Hinge2-CH2-CH3, and iv) a second light chain represented by VL2-C2, wherein C1 and C2 are capable of forming a dimer comprising at least one non-native inter-chain bond, and the two hinge regions and/or the two CH3 domains are capable of forming one or more inter-chains bond that can facilitate dimerization; wherein VH1-CH1 portion of i) and VL1-CL form an anti-CD3 arm (referred to as T3, see FIG. 1), and VH2-C1 of iii) and VL2-C2 form an anti-EGFR arm (referred to as U1, see FIG. 1).

### 2.3 Preparation of Bispecific Antibody for In Vivo Studies

The nucleic acid sequences encoding VL and VH of anti-CD3 antibody were amplified by PCR from an anti-CD3 monoclonal antibody (W3311-2.306.4-z1-uIgG1K). The VL and VH of anti-EGFR antibody were from ABX (Panitumumab), and their encoding nucleic acid sequences were synthesized by Genewiz Inc, respectively. CAlpha and CBeta genes were synthesized by Genewiz Inc. Anti-CD3 Native or Anti-EFGR chimeric light chain genes were inserted into a linearized vector containing a CMV promoter and a kappa signal peptide. The DNA fragments of Anti-CD3 VH-CH1 were inserted into a linearized vector containing human IgG4V9 (mutation S228P, F234A and L235A) constant region CH2-CH3 with a knob mutation. The DNA fragments of Anti-EGFRVH-Cbeta were inserted into a linearized vector containing human IgG4V9 (mutation S228P, F234A and L235A) constant region CH2-CH3 with a hole mutation. The vector contains a CMV promoter and a human antibody heavy chain signal peptide.

The resultant recombinant expression plasmids encoding heavy chain and light chain were co-transfected into Expi293 cells using Expi293 expression system kit (ThermoFisher-A14635) according to the manufacturer's instructions. Five days after transfection, the supernatants were collected, and the protein was purified by using Protein A column (GE Healthcare-17543 802) and further purified by using size exclusion column (GE Healthcare-17104301). Antibody concentration was measured by Nano Drop. The purity of proteins was evaluated by SDS-PAGE and HPLC-SEC.

### Results:

Anti-CD3×EGFR antibody, W3448-T3U1.E17R-1.uIgG4V9, was designed, constructed and produced. The format of the bispecific antibody was showed in FIG. 1, the SDS-PAGE and SEC-HPLC chromatogram were showed in FIG. 2 and FIG. 3, respectively. The expression titer of antibody W3448-T3U1.E17R-1.uIgG4V9 was higher than 170 mg/L through transient expression and with purity reaches 93.82%.

### Example 3

### In Vitro Characterization of the Bispecific Antibody

### 3.1 Human CD3 and EGFR Binding Activity Measured by FACS

Binding of EGFR×CD3 bispecific antibodies to human CD3 and EGFR expressing cells was determined by flow cytometry.

Briefly, 1×10⁵ Jurkat.2B8 (CD3+/EGFR−) or A431 (EGFR+/CD3−) cells were incubated for 60 minutes at 4° C. with serial dilutions of EGFR×CD3 bispecific antibody or hIgG4 isotype control antibodies. After washing twice with cold PBS supplemented with 1% bovine serum albumin (wash buffer), cell surface bound antibody was detected by incubating the cells with Fluorescence-labeled anti-human IgG antibody for 30 minutes at 4° C. Cells were washed twice in the same buffer and the mean fluorescence (MFI) of stained cells was measured using a FACS Canto II cytometer (BD Biosciences). Wells containing no antibody or secondary antibody only were used to establish background fluorescence. Four-parameter non-linear regression analysis was used to obtain EC₅₀ values for cell binding using GraphPad Prism software.

### Results:

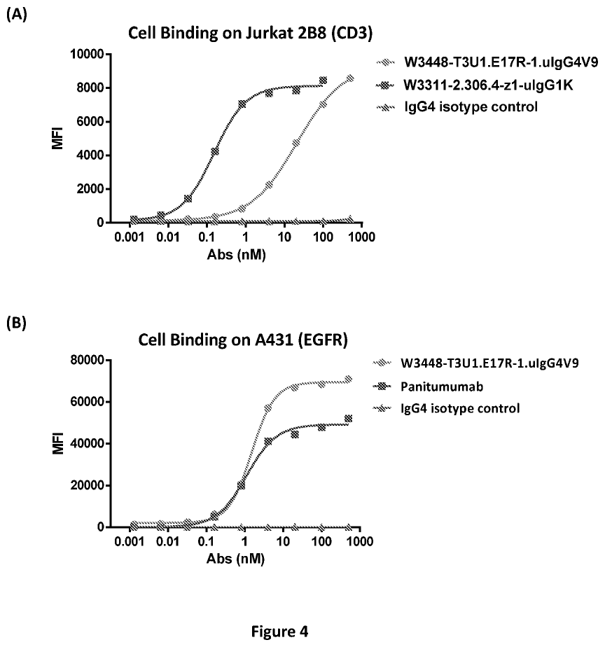

The binding activity of the lead BsAb (i.e., W3448-T3U1.E17R-1.uIgG4V9) to human CD3 and EGFR was performed by FACS using Jurkat.2B8 and A431 cell lines. The results were shown in FIG. 4 and Table 2. The data indicated that lead BsAb could bind to human CD3 with moderate affinity (i.e., as shown in FIG. 4A, the binding activity of the lead BsAb to Jurkat.2B8 (CD3+/EGFR−) cells was lower than the humanized anti-CD3 antibody, W3311-2.306.4-z1-uIgG1K), while showed strong binding activity to human EGFR-expression cells (i.e., as shown in FIG. 4B, the binding activity of the lead BsAb to A431 (EGFR+/CD3−) cells was higher than the Anti-EGFR antibody, Panitumumab).

TABLE 2
Binding activity of the lead BsAb to Human CD3 and EGFR

| | Jurkat.2B8 | | A431 | |
|---|---|---|---|---|
| | (CD3+/EGFR−) | | (EGFR+/CD3−) | |
| | cells | | cells | |
| | EC₅₀ | Top | EC₅₀ | Top |
| Abs | (nM) | MFI | (nM) | MFI |
| W3448-T3U1.E17R-1.uIgG4V9 | 20.83 | 8577 | 1.5 | 71000 |
| W3311-2.306.4-z1-uIgG1K | 0.15 | 8459 | / | / |
| Panitumumab | / | / | 1.11 | 52100 |
| IgG4 isotype control | NA | 235 | NA | 137 |

NA: not analyzed.

### 3.2 Cynomolgus Monkey CD3 and EGFR Cross Binding Activity Measured by FACS

Binding of EGFR×CD3 bispecific antibodies to cynomolgus monkey CD3 and EGFR expressing cells was determined by flow cytometry.

Briefly, 1×10⁵ cynomolgus monkey PBMC(CD3+/EGFR−) cells or CHOK1-cynoPro1 (EGFR+/CD3−) cells were incubated for 60 minutes at 4° C. with serial dilutions of EGFR×CD3 bispecific or hIgG4 isotype control antibodies. After washing twice with cold PBS supplemented with 1% bovine serum albumin (wash buffer), cell surface bound antibody was detected by incubating the cells with Fluorescence-labeled anti-human IgG antibody for 30 minutes at 4° C. Cells were washed twice in the same buffer and the mean fluorescence (MFI) of stained cells was measured using a FACS Canto II cytometer (BD Biosciences). Wells containing no antibody or secondary antibody only were used to establish background fluorescence. Four-parameter non-linear regression analysis was used to obtain EC₅₀ values for cell binding using GraphPad Prism software.

### Results:

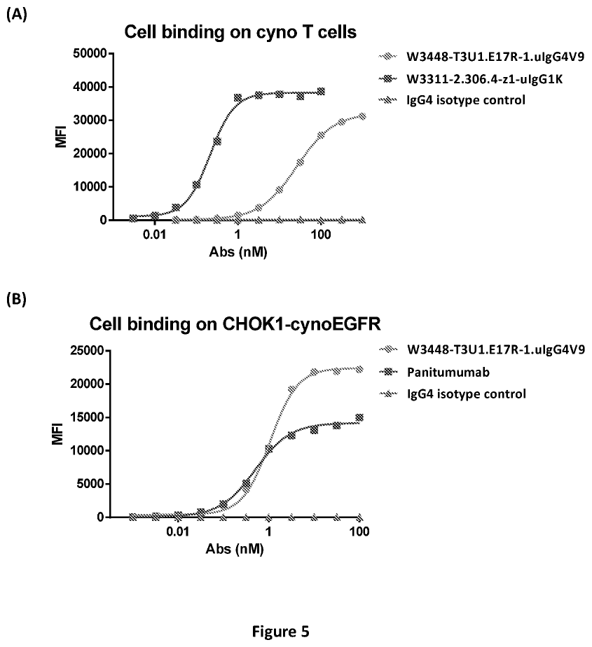

The binding activity of the lead BsAb (i.e., W3448-T3U1.E17R-1.uIgG4V9) to cynomolgus monkey CD3 and EGFR was performed via FACS by using cynomolgus monkey PBMC cells and EGFR-expressing stable CHOK1 cells. The results were showed in FIG. 5 and Table 3. The data indicated that the lead BsAb could bind to cyno CD3 with moderate affinity (i.e., as shown in FIG. 5A, the binding activity of the lead BsAb to cyno T cells was lower than the humanized anti-CD3 antibody, W3311-2.306.4-z1-uIgG1K), while showed strong binding activity to cyno EGFR-expression cells (i.e., as shown in FIG. 5B, the binding activity of the lead BsAb to CHOK1-cynoPro1 (EGFR+/CD3−) cell was higher than the Anti-EGFR antibody, Panitumumab).

TABLE 3
Binding activity of the lead BsAb to cyno CD3 and EGFR

| | | | CHOK1-cynoPro1 | |
|---|---|---|---|---|
| | Cyno T cells | | cells | |
| | EC₅₀ | Top | EC₅₀ | Top |
| Abs | (nM) | MFI | (nM) | MFI |
| W3448-T3U1.E17R-1.uIgG4V9 | 26.35 | 31200 | 1.07 | 22200 |
| W3311-2.306.4-z1-uIgG1K | 0.21 | 38700 | / | / |
| Panitumumab | / | / | 0.5 | 15000 |
| IgG4 isotype control | NA | 210 | NA | 38 |

NA: not analyzed.

### 3.3 Human CD3 and EGFR Binding Affinity Measured by FACS

Binding affinity of EGFR×CD3 bispecific antibodies to human CD3 and EGFR was measured by FACS analysis. A431 (EGFR+/CD3−) cells and Jurkat.2B8 (CD3+/EGFR−) cells were transferred in to 96-well U-bottom plates at a density of 5×104 cells/ml, respectively. Tested antibodies were serially diluted in wash buffer (1×PBS/1% BSA) and incubated with cells at 4° C. for 1 h. The secondary antibody goat anti-human IgG Fc FITC (Jackson, 109-095-098, 3.6 moles FITC per mole IgG) was added and incubated at 4° C. in the dark for 0.5 h. The cells were then washed once and re-suspended in 1×PBS/1% BSA, and analyzed by flow cytometry. Fluorescence intensity will be converted to bound molecules/cell based on the quantitative beads (Quantum™ MESF Kits, Bangs Laboratories, Inc.).

### Results:

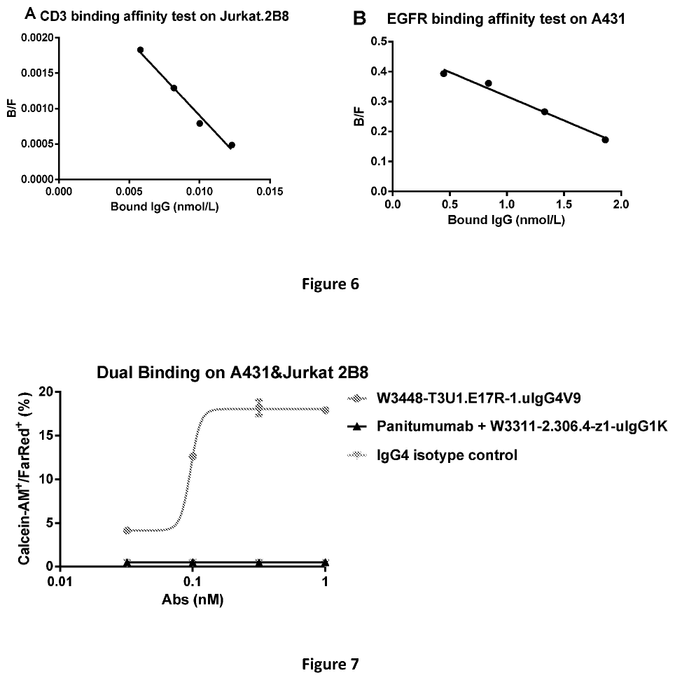

The binding affinity of W3448-T3U1.E17R-1.uIgG4V9 to human CD3 and EGFR was tested on Jurkat.2B8 cells and A431 cells by flow cytometry. As showed in FIG. 6 and Table 4, the fitted KD values of binding to CD3 (showed in FIG. 6A) and EGFR (showed in FIG. 6B) were 4.7 nM and 6.2 nM, respectively.

TABLE 4
Binding affinity of the lead BsAb to human CD3 and EGFR

| Sample | Jurkat.2B8 (hCD3) | A431 (hEGFR) |
|---|---|---|
| Bmax (M) | 1.40 × 10⁻¹¹ | 3.00 × 10⁻⁹ |
| KD (M) | 4.70 × 10⁻⁹ | 6.20 × 10⁻⁹ |
| r² | 0.98 | 0.98 |

### 3.4 Dual Binding on Target Cells Measured by FACS

For testing the simultaneous binding of the lead BsAb to human CD3 and EGFR expressing cells, 1×10⁶/ml A431 (EGFR+/CD3−) cells and 1×10⁶/ml Jurkat.2B8 (CD3+/EGFR−) cells were labeled with 50 nM Calcein-AM (Invitrogen-C3099) and 20 nM FarRed (Invitrogen-C34572), respectively. After washing with cold 1% BSA/1×PBS, the labelled A431 and Jurkat.2B8 cells were resuspended and mixed to a final concentration of 1×10⁶/ml at the ratio of 2:3 (ratio of cell number). 1×10⁵/well of the mixed cells were plated and serial dilutions of BsAb was then added. After incubation at 4° C. for 60 minutes, the percentage of Calcein-AM and FarRed double positive cells was analyzed by FACS.

### Results:

The bridging binding activity of W3448-T3U1.E17R-1.uIgG4V9 to CD3 and EGFR-expressing cells was tested using pre-labeled Jurkat.2B8 and A431 cells by flow cytometry. As showed in FIG. 7, compared with the negative control, roughly 18% of the population of bridged Jurkat.2B8 and A431 cells was bridged through bispecific antibody W3448-T3U1.E17R-1.uIgG4V9.

### 3.5 Assessment of Human T Cell Activation

Freshly isolated human PBMCs were used as effector cells and activated by EGFR×CD3 bispecific antibodies as measured by the induction of CD69 and CD25 surface expression. PBMCs were freshly isolated by Ficoll-Paque PLUS (GE Healthcare, #17-1440-03) density centrifugation from heparinized venous blood and then cultured overnight in complete media (RPMI 1640 supplemented with 10% FBS, 100 U/ml penicillin, and 100 μg/ml streptomycin). On the assay day, PBMCs with or without A431, HT-29 and HCC1419 (Target cells: 1×10⁴ cells/well; E:T Ratio, 10:1) and antibodies in RPMI 1640/10% FBS were co-incubated at 37° C. for 24 hours. After washing once with 1% BSA, the cell pellets were re-suspended with staining buffer containing anti-human Ab panel (FITC labeled anti-human CD4 (BD Pharmingen-550628); PerCP-Cy5.5 labeled anti-human CD8 (BD Pharmingen-560662); PE labeled anti-human CD69 (BD Pharmingen-555531) and APC labeled anti-human CD25 (BD Pharmingen-555434)). The cells were washed twice with 1% BSA after 30 minutes incubation at 4° C. The percentage of PE or APC positive cells in FITC or PerCp-Cy5.5 positive cells was determined by flow cytometry (BD Biosciences).

T cell activation by BsAbs was determined by flow cytometry measuring the percentage of CD69 or CD25 expressing effector cells. Freshly isolated purified human CD4⁺ T cells and CD8⁺ T cells were examined as effector cells, respectively. Briefly, 5×10⁴ CD4⁺ T or CD8⁺ T cells were plated in 110 μl/well complete media containing serial dilution of BsAbs or hIgG4 isotype control antibody, in the presence or absence of 1×10⁴ A431, HT-29 or HCC1419 cells/well for 24 hours at 37° C. After incubation, the cells were washed twice with 1% BSA/1×DPBS and then stained with anti-human Ab panel (FITC labeled anti-human CD4 (BD Pharmingen-550628); PerCP-Cy5.5 labeled anti-human CD8 (BD Pharmingen-560662); PE labeled anti-human CD69 (BD Pharmingen-555531) and APC labeled anti-human CD25 (BD Pharmingen-555434)) at 4° C. for 30 minutes. T cell activation evaluated by CD69 or CD25 expression was analyzed by FACS. EC50 of T-cell activation was determined by using Prism four-parameter non-linear regression analysis.

### Results:

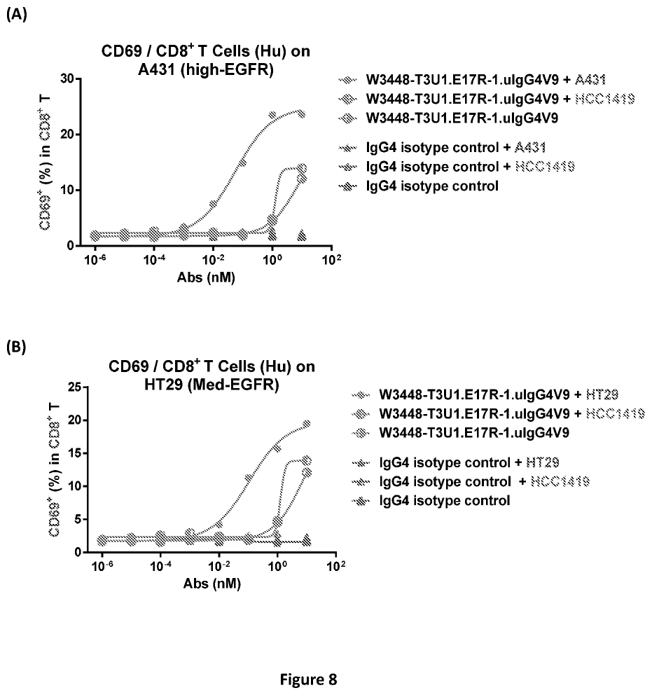

To test W3448-T3U1.E17R-1.uIgG4V9 human T cell activation, tumor cells A431 (high EGFR expression), HT-29 (medium EGFR expression) and HCC1419 (negative EGFR expression) were used as target cells. As showed in FIG. 8 and Table 5, in human PBMC T cell activation assay, W3448-T3U1.E17R-1.uIgG4V9 potently induced CD8+T-cell activation in the presence of EGFR-expressing cells (A431 and HT-29), whereas there was much lower extent T-cell activation in the presence of EGFR negative expressing cells (HCC1419).

TABLE 5
Human T cell activation in the presence of tumor cells

| | CD69 on CD 8+ T cells (human) | | | | | | | |
|---|---|---|---|---|---|---|---|---|
| | CD 8+ T + A431 | | CD 8+ T + HT29 | | CD 8+ T + HCC1419 | | | |
| | (high) | | (medium) | | (negative) | | CD 8+ T only | |
| Abs | EC₅₀ (nM) | Max % | EC₅₀ (nM) | Max % | EC₅₀ (nM) | Max % | EC₅₀ (nM) | Max % |
| W3448-T3U1.E17R- | 0.055 | 23.6 | 0.107 | 19.5 | NA | 13.9 | NA | 12.1 |
| 1.uIgG4V9 | | | | | | | | |
| IgG4 isotype control | >10 | 2.3 | >10 | 2.2 | NA | 2.8 | NA | 2.1 |

NA: not analyzed.

### 3.6 Cynomolgus Monkey T Cell Activation on Tumor Cells

To test cynomolgus monkey T cell activation of W3448-T3U1.E17R-1.uIgG4V9, tumor cells A431 (high EGFR expression), HT-29 (medium EGFR expression) and HCC1419 (negative EGFR expression) were used was target cells. The procedures are similar to Section 3.5.

### Results:

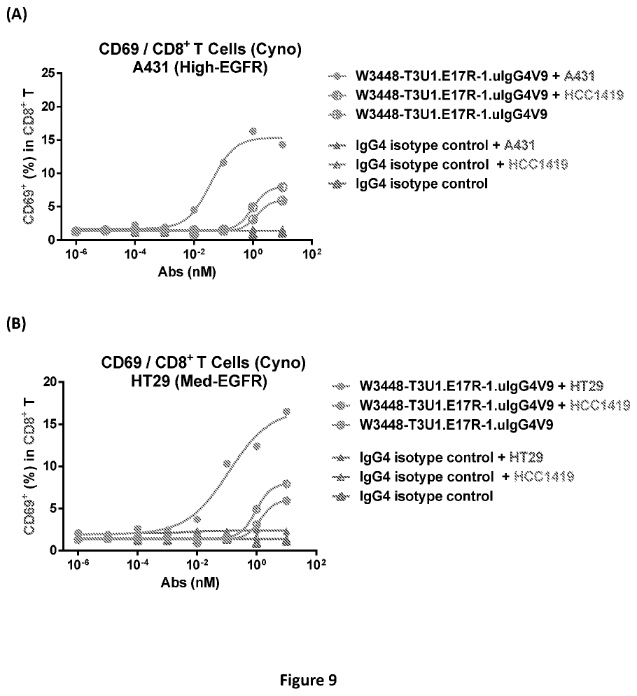

As showed in FIG. 9 and Table 6, in monkey PBMC T cell activation assay, W3448-T3U1.E17R-1.uIgG4V9 potently induced CD8+T-cell activation in the presence of EGFR-expressing cells (A431 and HT-29), whereas there was much lower extent T-cell activation in the presence of EGFR negative expressing cells (HCC1419).

TABLE 6
Cynomolgus monkey T cell activation in the presence of tumor cells

| | CD69 on CD 8+ T cells (cyno) | | | | | | | |
|---|---|---|---|---|---|---|---|---|
| | CD 8+ T + A431 | | CD 8+ T + HT29 | | CD 8+ T + HCC1419 | | | |
| | (high) | | (medium) | | (negative) | | CD 8+ T only | |
| Abs | EC₅₀ (nM) | Max % | EC₅₀ (nM) | Max % | EC₅₀ (nM) | Max % | EC₅₀ (nM) | Max % |
| W3448-T3U1.E17R- | 0.037 | 16.3 | 0.115 | 16.5 | NA | 7.9 | NA | 5.9 |
| 1.uIgG4V9 | | | | | | | | |
| IgG4 isotype control | >10 | 1.6 | 0.005 | 2.5 | NA | 1.7 | NA | 1.4 |

NA: not analyzed.

### 3.7 Cytotoxicity Assay Against Tumor Cells

Pre-activated human PBMCs were used as effector cells to determine the ability of the EGFR×CD3 bispecific antibodies to mediate specific tumor cell lysis in a luciferase quantitative assay. In brief, isolated human PBMCs were cultured for 3 days in complete media (RPMI1640 supplemented with 10% FBS, 100 U/ml penicillin, 100 μg/ml streptomycin) containing 50 IU/ml recombinant human IL-2 (Delusheng, #20171166b) and 10 ng/ml OKT-3 (eBioscience, #16-0037-85). On the assay day, target cells were seeded in duplicate in 96-well microplates with effector cells (PBMC/target cell ratio 10:1) and serial dilution of bispecific antibodies or parental monoclonal antibodies in complete media. Effector and tumor cells were allowed to interact at 37° C. for 3 days. On the test day, plates were washed with 180 μL/well DPBS twice, and then added 50 μL/well Cell Titer Glo reagent (Promega, #G7573). The bioluminescent signal was measured by Envision (PerkinElmer) after 10 minutes incubation in dark.

Percent cytotoxicity was calculated using the equation:

% cytotoxicity=(Luc S−Luc Effector cell only)/(Luc Tumor cell only−Luc Effector cell only)*100%

wherein “Luc S” is the bioluminescent signal of the test well, “Luc Effector cell only” is bioluminescent signal of the residual effector cells and “Luc Tumor cell only” is bioluminescent signal of tumor cell only control well.

Results are expressed as the % specific lysis (mean±SD) from duplicate wells.

### Results:

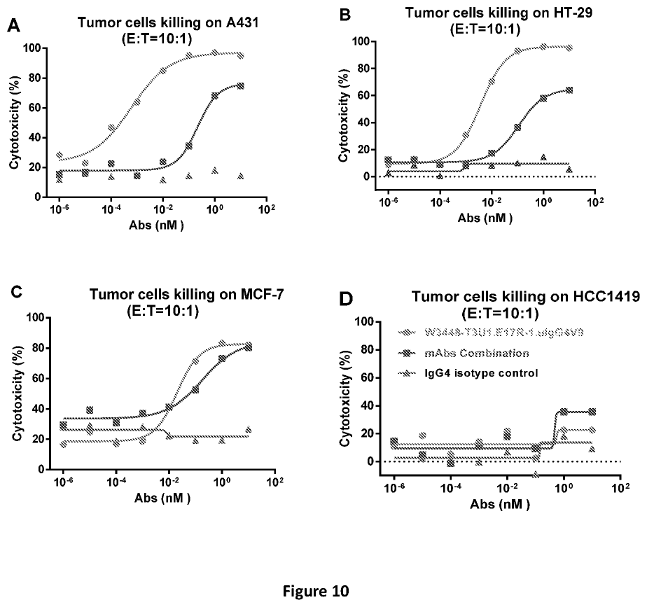

The cytotoxicity activity of W3448-T3U1.E17R-1.uIgG4V9 was evaluated using four varying EGFR-expressing tumor cells in the presence of primary T cells. As showed in FIG. 10 and Table 7, the lead BsAb showed selective and more potent cytotoxicity on EGRF-expressing tumor cells, but only negligible killing on EGFR negative cells. The cytotoxicity with an EC50 value from 0.6 to 19.88, which was accordance with EFGR expression on tumor cells.

TABLE 7
T cells killing against tumor cells

| | A431 (high EGFR | | HT-29 (medium | | MCF-7 (low EGFR | | HCC1419 (No | |
|---|---|---|---|---|---|---|---|---|
| | expression) | | EGFR expression) | | expression) | | EGFR expression) | |
| | IC50 | Max | IC50 | Max | IC50 | Max | IC50 | Max |
| Abs | (pM) | Cyto % | (pM) | Cyto % | (pM) | Cyto % | (pM) | Cyto % |
| W3448-T3U1.E17R-1.uIgG4V9 | 0.6 | 97.1 | 3.7 | 96.2 | 19.88 | 83.1 | NA | 22.5 |
| W3311-2.306.4-z1-uIgG1K + | 214.9 | 74.8 | 111.5 | 64.1 | 181 | 80.3 | NA | 35.6 |
| Panitumumab | | | | | | | | |
| IgG4 isotype control | NA | 18 | NA | 14.5 | NA | 31.3 | NA | 18.2 |

NA: not analyzed.

### 3.8 ADCC and CDC Assays

Antibody dependent cell-mediated cytotoxicity (ADCC) or complement-dependent cytotoxicity (CDC) was determined by a LDH release assay. Human peripheral blood mononuclear cells (PBMCs) were freshly isolated by Ficoll-Paque PLUS (GE Healthcare, #17-1440-03) density centrifugation from heparinized venous blood and then cultured overnight in complete media (RPMI1640 supplemented with 10% FBS, 100 U/ml penicillin, 100 μg/ml streptomycin). NK cells were isolated by Human CD56 Positive Selection Kit (Miltenyi-130-050-401). In brief, on the day of the ADCC assay, EGFR expressing target cells A431 or CD3 expressing target cell Jurkat.2B8 (2E⁴/well) were plated in 110 μL with effector cells (NK/target cell ratio 2.5:1) and serial dilution of antibodies or hIgG isotype control in complete media for 4 hours at 37° C. Following incubation, the plates were centrifuged and 70 μL supernatants were transferred to a clear bottom 96-well plate (Corning, #3599) and 50 μL reaction mixture (Roche, #116447930, Cytotoxicity Reaction Kit) was added to each well and incubate for 15 minutes. After adding stop solution, plates were read by M5e to measure the absorbance of the samples at 492 nm and 600 nm.

Percent cytotoxicity was calculated using the equation:

% cytotoxicity=(Sample−Effector cell control−target cell control)/(target cell lysis−target cell control)*100%

For CDC assay, EGFR expressing target cells A431 or CD3 expressing target cell Jurkat.2B8 (2E⁴/well) were plated in 110 μL with human normal serum (final 1:50 diluted) (Quidel, #A113) and serial dilution of antibodies or hIgG isotype control in complete media for 2 hours at 37° C. Following incubation, the plates were centrifuged and 70 μL supernatants were transferred to a clear bottom 96-well plate (Corning, #3599) and 50 μL reaction mixture (Roche, #116447930, Cytotoxicity Reaction Kit) was added to each well and incubate for 15 minutes. After adding stop solution, plates were read by M5e to measure the absorbance of the samples at 492 nm and 600 nm.

Percent cytotoxicity was calculated using the equation:

% cytotoxicity=(Sample−target cell control)/(Target Cell lysis−target cell control)*100%

The IC₅₀ values for killing were determined using GraphPad Prism software with values calculated using a four-parameter non-linear regression analysis.

### Results:

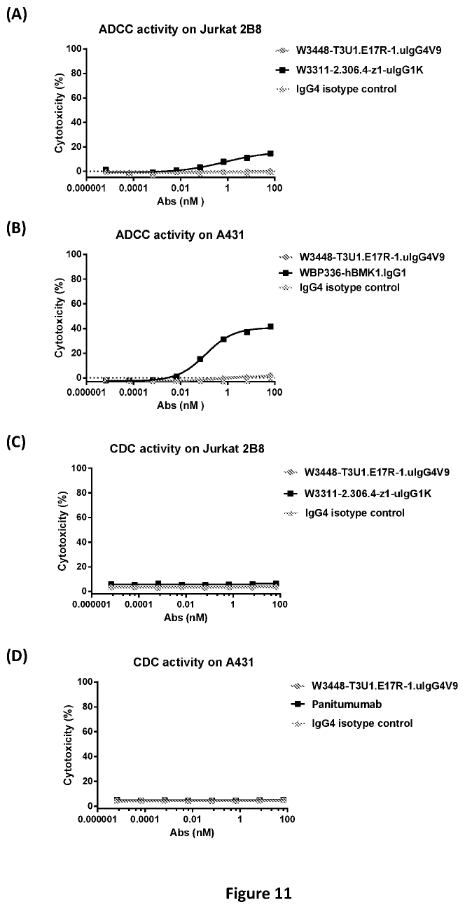

The cead antibody, W3448-T3U1.E17R-1.uIgG4V9 was evaluated for its ADCC and CDC ability on Jurkat. 2B8 and A431 cells. As showed in FIG. 11, the lead BsAb induced no ADCC and CDC activity both on Jurkat. 2B8 and A431 cells tumor cells.

### 3.9 Thermal Stability (DSF)

Tₘ of antibodies was investigated using QuantStudio™ 7 Flex Real-Time PCR system (Applied Biosystems).

Briefly, 19 μL of antibody solution was mixed with 1 μL of 62.5×SYPRO Orange solution (Invitrogen) and transferred to a 96-well plate (Biosystems). The plate was heated from 26° C. to 95° C. at a rate of 0.9° C./min, and the resulting fluorescence data was collected. The negative derivatives of the fluorescence changes with respect to different temperatures were calculated, and the maximal value was defined as melting temperature Tₘ. If a protein has multiple unfolding transitions, the first two Tₘ were reported, named as Tₘ1 and Tₘ2, respectively. Data collection and Tₘ calculation were conducted automatically by the operation software (QuantStudio™ Real Time PCR software v1.3).

### Results:

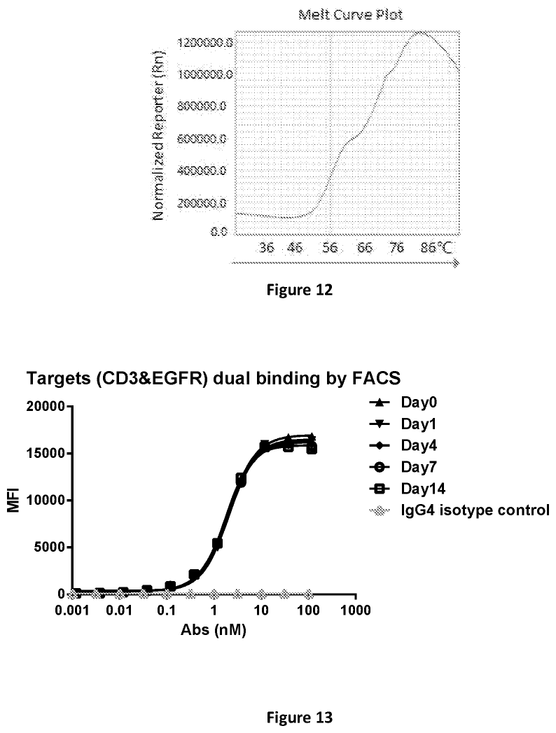

Differential scanning fluorometry (DSF) was used to evaluate W3448-T3U1.E17R-1.uIgG4V9 thermal stability. As showed in Table 8 and in FIG. 12, Tₘ1 and Tₘ2 of W3448-T3U1.E17R-1.uIgG4V9 were 55.4° C. and 71.8° C., respectively.

TABLE 8
Thermal stability parameters of DSF assay

| Protein Name | pI | Buffer | Tₘ1 (° C.) | Tₘ2 (° C.) |
|---|---|---|---|---|
| W3448-T3U1.E17R- | 6.1 | 50 mM Sodium | 55.4 | 71.8 |
| 1.uIgG4V9 | | Acetate, 7% Sucrose, | | |
| | | pH 5.6 | | |

### 3.10 Serum Stability

For dual binding FACS analysis, the samples from different time points were free-thawed simultaneously at 4° C. The thawed antibodies were serially diluted and added to 1×10⁵ A431 cells/well and incubated for 1 hour at 4° C. The cells were washed twice with PBS supplemented with 1% bovine serum albumin. W331-hPro1.ECD.His (Sino) (3.16 nM) were added to the cells and incubated at 4° C. for 1 hour. After 1-hour incubation, cells are washed twice with PBS supplemented with 1% bovine serum albumin, then the His Tag Antibody [Biotin], mAb, Mouse (GenScript-A00613) diluted 1:400 in 1% bovine serum albumin were added to the cells and incubated at 4° C. for 30 minutes. After the cells were washed twice with 1% bovine serum albumin, Alexa647-conjugated Streptavidin (Jackson Immuno Research-016-600-084) diluted 1:4000 with 1% bovine serum albumin were added to the cells and incubated at 4° C. for 30 minutes. Cells were washed twice in the same buffer and the mean fluorescence (MFI) of stained cells was measured using a FACS Canto II cytometer (BD Biosciences) and analyzed by FlowJo. Wells containing no antibody or secondary antibody only were used to establish background fluorescence. Four-parameter non-linear regression analysis was used to obtain EC50 values for cell binding using GraphPad Prism software.

### Results:

Serum stability of W3448-T3U1.E17R-1.uIgG4V9 was performed in human serum. The lead BsAb, W3448-T3U1.E17R-1.uIgG4V9 was co-cultured with human serum at 37° C. for 0, 1, 4, 7 and 14 days and the binding activity was tested by FACS. As showed in FIG. 13, the data showed serum culturing had no adverse effect on W3448-T3U1.E17R-1.uIgG4V9 binding ability to CD3 and EGFR.

### Example 4

### In Vivo Anti-Tumor Efficacy Study in Human PBMC-HT29 Model

Anti-tumor efficacy of W3448-T3U1.E17R-1.uIgG4V9 was tested in human PBMC-HT29 model in NCG female mice (Nanjing Galaxy Biopharmaceutical Co.). Female NCG mice of 13-14-week-old (purchased from NCG) were used in the study. HT29 cells were maintained in vitro as a monolayer culture in RPMI 1640 medium supplemented with 10% fetal bovine serum, 100 U/mL penicillin and 100 μg/mL streptomycin at 37° C. in an atmosphere of 5% CO₂ in air. The tumor cells were routinely sub-cultured twice a week with 0.25% trypsin-EDTA treatment. The cells growing in an exponential growth phase were harvested and counted for tumor inoculation.

For the therapeutic model establishment, on Day 0, each mouse was inoculated subcutaneously at the right front flank with HT29 tumor cells (2.0×10⁶ cells in 100 ul PBS) and injected 2.0×10⁶ PBMC cells (Hemacare, Lot Number: 19054078) in 100 ul PBS intraperitoneally. On Day 11 post PBMC implantation, peripheral human/mouse CD45, Human CD3 was detected by FACS. Animals with absolute human CD3>3% were selected for following study; On Day 12 post PBMC implantation, when the average tumor volume reached approximately to 130 mm³, animals were randomly grouped into 4 groups and each group contained 5 mice. The 4 groups of mice received following injections intraperitoneally weekly for total 4 injections, respectively: Isotype control at 0.3 mg/kg body weight; 0.3 mg/kg body weight of Panitumumab; 0.3 mg/kg body weight of W3448-T3U1.E17R-1.uIgG4V9; 0.08 mg/kg body weight of W3448-T3U1.E17R-1.uIgG4V9. The day of the first injection was considered as day 0. For all tumor studies, mice were weighed, and tumor growth was measured twice a week using calipers. Peripheral human CD45, human CD3 were detected periodically. All the procedures related to animal handling, care and the treatment in the study were performed according to the guidelines approved by the Institutional Animal Care and Use Committee (IACUC) of Shanghai SIPPR-BK Laboratory Animal Co., Ltd following the guidance of the Association for Assessment and Accreditation of Laboratory Animal Care (AAALAC). Tumor volume was calculated with the formula (12 (length×width²). TGI (tumor growth inhibition) was calculated for each group using the formula:

TGI (%)=[1−(Ti−T0)/(Vi−V0)]×100.

Ti is the average tumor volume of a treatment group on a given day. T0 is the average tumor volume of the treatment group on the first day of treatment. Vi is the average tumor volume of the vehicle control group on the same day with Ti and V0 is the average tumor volume of the vehicle group on the first day of treatment.

The results were represented by mean and the standard error (Mean±SEM). Data were analyzed using Two way ANOVA Tukey's multiple comparison test with Graphpad Prism 6.0 and p<0.05 was considered to be statistically significant.

### Results:

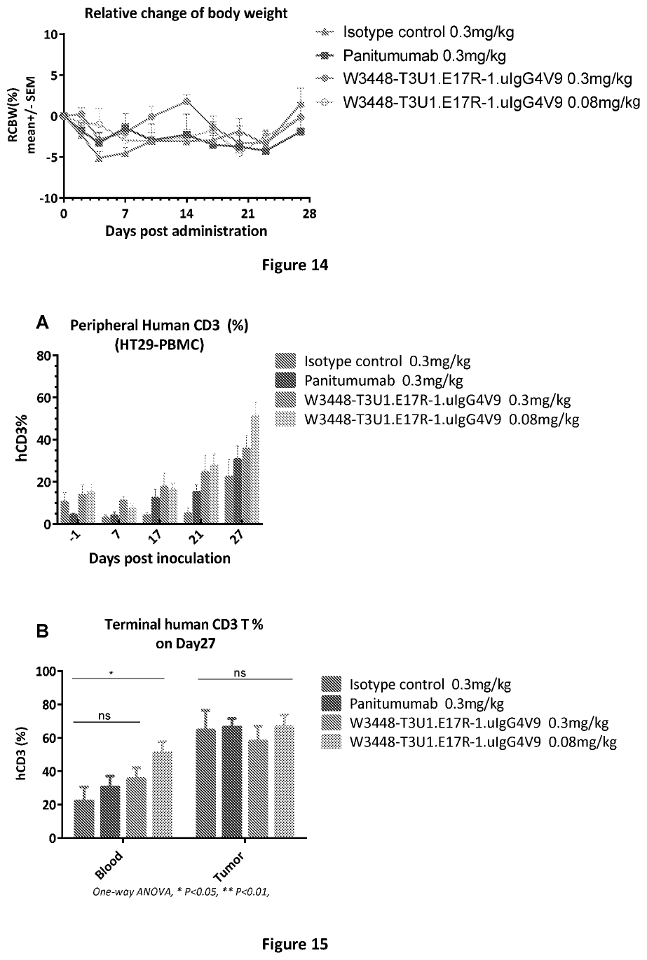

All mice were survived over the period of study. Two animals (one in Isotype control and one in 0.3 mg/kg body weight of W3448-T3U1.E17R-1.uIgG4V9 showed obvious bodyweight loss (>15%), which were identified as GVHD (graft-versus-host disease) and excluded in final data analysis. As showed in FIG. 14, overall mean bodyweight loss in all groups were within 5%, without statistical difference observed among each group (p>0.05, Two way ANOVA).

As showed in FIG. 15A, the percentage of peripheral human CD3 were monitored over the period of study. No obvious T cell depletion was observed in W3448-T3U1.E17R-1.uIgG4V9 groups at 0.08 mg/kg and 0.3 mg/kg. As showed in FIG. 15B, on Day 27 post treatment, comparing to the isotype control, significant increase in peripheral human CD3 was observed in low dose of W3448-T3U1.E17R-1.uIgG4V9 groups (0.08 mg/kg body weight, p<0.01, One-way ANOVA); in tumor tissue, no difference in human CD3 was observed among all groups.

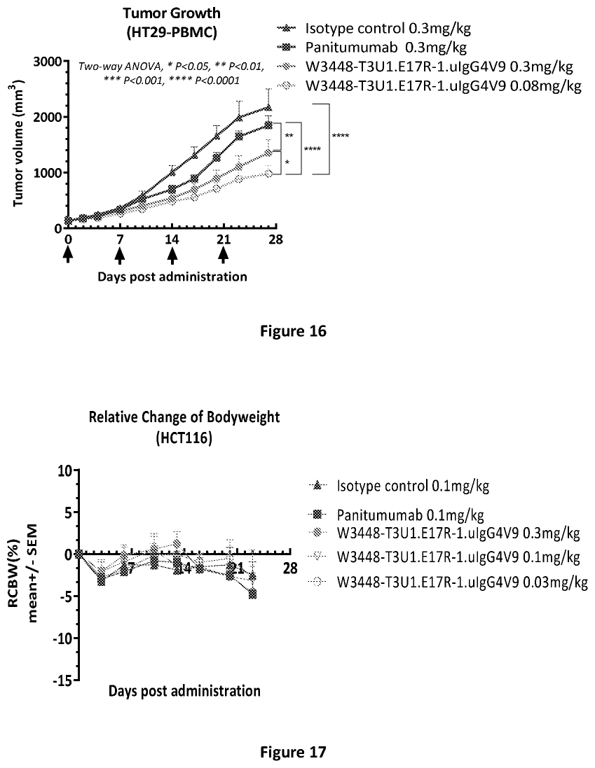

As showed in FIG. 16, on Day 27 post the first dosing, the mean tumor volume of Isotype control group was 2174 mm³, which indicated HT29 model was well established. Compared with Isotype group, Panitumumab at 0.3 mg/kg body weight did not inhibit tumor growth (p>0.05); W3448-T3U1.E17R-1.uIgG4V9 significantly inhibited tumor growth at 0.08 mpk and 0.3 mpk (p<0.0001 and p<0.001, respectively). Comparing to Panitumumab at 0.3 mg/kg body weight, W3448-T3U1.E17R-1.uIgG4V9 at 0.3 mg/kg body weight significantly inhibited tumor growth (p<0.01), W3448-T3U1.E17R-1.uIgG4V9 at 0.08 mg/kg body weight significantly inhibited tumor growth (p<0.0001). The TGI at day 27 of each group was 16.21% for Panitumumab at 0.3 mg/kg body weight, 33.35% for W3448-T3U1.E17R-1.uIgG4V9 at 0.3 mg/kg body weight, and 58.61% for W3448-T3U1.E17R-1.uIgG4V9 at 0.08 mg/kg body weight, showed in Table 9.

The data in FIG. 16 and Table 9 showed that W3448-T3U1.E17R-1.uIgG4V9 unexpectedly achieved higher TGI at lower dose 0.08 mg/kg than the TGI at 0.3 mg/kg.

TABLE 9
Tumor growth inhibition

| | Tumor growth inhibition | | |
|---|---|---|---|
| | (TGI %, Day) | | |
| | (Compared with isotype control) | | |
| Abs | Day 14 | Day 20 | Day 27 |
| Panitumumab, 0.3 mg/kg | 35.5 | 25.76 | 16.21 |
| W3448-T3U1.E17R-1.uIgG4V9, | 41.72 | 42.49 | 33.35 |
| 0.3 mg/kg | | | |
| W3448-T3U1.E17R-1.uIgG4V9, | 59.96 | 62.16 | 58.61 |
| 0.08 mg/kg | | | |

### Example 5

### In Vivo Anti-Tumor Efficacy Study in Human PBMC-HCT116 NCG Mouse Model

Anti-tumor efficacy of W3448-T3U1.E17R-1.uIgG4V9 was investigated in human PBMC-HCT116-NCG mouse model (Nanjing Galaxy Biopharmaceutical Co.). 10-11 week-old female NCG mice were used in the study. HCT116 cells were maintained in vitro as a monolayer culture in McCoy's 5A medium supplemented with 10% fetal bovine serum, 100 U/mL penicillin and 100 μg/mL streptomycin at 37° C. in an atmosphere of 5% CO₂ in air. The tumor cells were routinely sub-cultured twice a week with 0.25% trypsin-EDTA treatment. The cells growing into an exponential growth phase were harvested and counted for tumor inoculation.

For the therapeutic model establishment, on Day 0, each mouse was inoculated subcutaneously at the right front flank with HCT116 tumor cells (1.5×10⁶ cells in 100 ul PBS) and intraperitoneally injected 3.0×106 PBMC cells (Hemacare, Lot Number: 19054078) in 100 ul PBS. On Day 11 post PBMC implantation, peripheral human/mouse CD45, human CD3 was detected by FACS. Animals with absolute human CD3>3% were selected for following study: on Day 11 post PBMC implantation, when the average tumor volume reached approximately to 120 mm³, animals were randomly grouped into 5 groups and each group contained 7 mice. The 5 groups of mice received following weekly intraperitoneal injections for total 4 injections, respectively: Isotype control at 0.1 mg/kg; 0.1 mg/kg of Panitumumab (WBP336-hBMK2.IgG2); 0.3 mg/kg of W3448-T3U1.E17R-1.uIgG4V9; 0.1 mg/kg of W3448-T3U1.E17R-1.uIgG4V9; 0.03 mg/kg of W3448-T3U1.E17R-1.uIgG4V9. The day of the first injection was indicated as day 0. For all tumor studies, mice were weighed and tumor growth was measured twice a week using calipers. Peripheral human CD45, human CD3 were detected periodically. All the procedures related to animal handling, care and the treatment in the study were performed according to the guidelines approved by the Institutional Animal Care and Use Committee (IACUC) of Shanghai SIPPR-BK Laboratory Animal Co., Ltd following the guidance of the Association for Assessment and Accreditation of Laboratory Animal Care (AAALAC). Tumor volume was calculated with the formula (12 (length×width²). TGI (tumor growth inhibition) was calculated for each group using the formula: TGI (%)=[1−(Ti−T0)/(Vi−V0)]×100, wherein Ti is the average tumor volume of a treatment group on a given day, T0 is the average tumor volume of the treatment group on the first day of treatment, Vi is the average tumor volume of the vehicle control group on the same day with Ti, and V0 is the average tumor volume of the vehicle group on the first day of treatment. The results were represented by mean and the standard error (Mean±SEM). Data were analyzed using Two way ANOVA Tukey's multiple comparisons test with Graphpad Prism 6.0 and p<0.05 was considered to be statistically significant.

### Results:

All mice were survived over the period of study. The proportion of peripheral blood lymphocyte reconstitution was measured by flow cytometry. Few CD45+ cells were confirmed in three animals, which were excluded in final data analysis (one in 0.3 mg/kg W3448-T3U1.E17R-1.uIgG4V9; one in 0.1 mg/kg of W3448-T3U1.E17R-1.uIgG4V9 and one in 0.03 mg/kg of W3448-T3U1.E17R-1.uIgG4V9). As shown in FIG. 17, overall mean bodyweight loss in all groups were not more than 5%, without statistical difference observed among each group (p>0.05, Two way ANOVA).

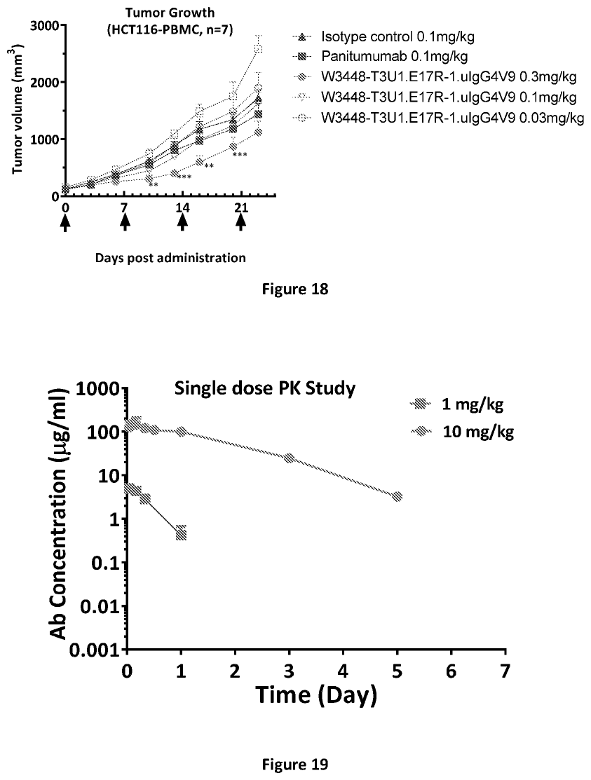

As shown in FIG. 18, on Day 23 post the first dosing, the mean tumor volume of Isotype control group was 1720 mm³, which indicated HCT116 model was well established. Compared with Isotype group, Panitumumab at 0.1 mg/kg did not inhibited tumor growth (p>0.05); W3448-T3U1.E17R-1.uIgG4V9 significantly inhibited tumor growth at 0.3 mg/kg (p<0.001), TGI was listed in table 10.

TABLE 10
Tumor growth inhibition

| | Tumor growth inhibition | | |
|---|---|---|---|
| | (TGI %, Day) | | |
| | (Compared with isotype control) | | |
| Abs | Day 13 | Day 16 | Day 20 |
| Panitumumab, 0.1 mg/kg | 10.28 | 19.32 | 13.32 |
| W3448-T3U1.E17R-1.uIgG4V9, | 56.22 | 46.69 | 29.72 |
| 0.3 mg/kg | | | |
| W3448-T3U1.E17R-1.uIgG4V9, | 24.98 | 16.09 | 4.97 |
| 0.1 mg/kg | | | |
| W3448-T3U1.E17R-1.uIgG4V9, | 1.20 | −11.06 | −18.51 |
| 0.03 mg/kg | | | |

### Example 6

### Single Dose PK and Preliminary Toxicity Study of W3448-T3U1.E17R-1.uIgG4V9 in Cynomolgus Monkeys

4 cynomolgus monkeys (2 animals/group) were divided into two groups. Animals in groups 1 and 2 were administered with W3448-T3U1.E17R-1.uIgG4V9 at 1 mg/kg and 10 mg/kg by single intravenous bolus administration, respectively. Animal information was listed in table 11. Cage-side observations for general health and appearance, especially skin irritation was performed. Whole blood sample analysis for hematology (CBC) and serum analysis for chemistry detection were determined by hematology analyzer (ADVIA2120) and chemistry (HITACHI 7180), respectively. Samples collection information was listed in table 12.

TABLE 11
Animal information

| | |
|---|---|
| Species | Macaca |
| Name | Cynomolgus Monkey |
| Sex | Female |
| Body weight | 2-2.24 kg |
| Ages | 3-4 years |
| Animal No. | 4 (1 mg/kg for C1501 and C1502; 10 mg/kg |
| | for C2501 and C2502) |
| Animal supplier: | Hainan Jingang Biotech Co., Ltd |

TABLE 12
Sample collection strategy

| | | | Shipping | | |
|---|---|---|---|---|---|
| Matrix | Detect factor | Volume | Destination | Group | Time-points |
| Whole blood | Hematology* | 500 μL | WuXi AppTec | Group | Pre-dose, Day 1 (24 h), Day 3 (72 h), |
| | | | (Suzhou) Co., Ltd. | 1~2 | Day 7 (168 h), Day 14 (336 h), Day 21 (504 h), |
| Serum | Clinical Chemistry* | 500 μL | | | Day 28 (672 h) |
| Serum | PK | 200 μL | WuXi Biologies | | Pre-dose, 1 h, 4 h, 8 h, 24 h, Day 3 (72 h), |
| | | | | | Day 5 (120 h), Day 7 (168 h), Day 10 (240 h), |
| | | | | | Day 12 (288 h), Day 14 (336 h), Day 21 (504 h), |
| | | | | | Day 28 (672 h) |
| Clinical | Twice daily (approximately 9:30 a.m. | NA | WuXi AppTec | | NA |
| observation | and 4:00 p.m.), Cage-side | | (Suzhou) Co., Ltd. | | |
| | observations for general health and | | | | |
| | appearance, especially skin irritation. | | | | |
| Serum | ADA | 500 μL | WuXi Biologies | | Pre-dose, Day 14 (336 h) and Day 28 (672 h) |
| Serum | Cytokine* | 100 μL | WuXi Biologies | | Pre-dose, 1 h, 4 h, 8 h, 24 h, Day 3 (72 h), |
| | | | | | Day 7 (168 h) |
| Whole blood | Immunophenotyping* | 1000 μL | WuXi Biologics | | Pre-dose, 24 h, Day 3 (72 h), Day 7 (168 h), |
| | | | | | Day 14 (336 h), Day 21 (504 h), Day 28 (672 h) |

Note:
Day 0 is the first day of dosing.
The extra serum samples for PK study were sent to Biologies for analysis.
*Cytokine: IL-2, IFN-γ, TNF-α, IL-4, IL-5, IL-6
*Immunophenotyping: CD45, CD3, CD4, CD8
*Clinical Chemistry: ALT, AST, ALP, GGT, CK, CRE, TP, ALB, GLB, A/G, TBIL, TCHO, TG, GLU, UREA, Ca, P, Na, K, Cl, LDH
*Hematology: WBC, RBC, HCT, MCV, RDW, HGB, MCH, MCHC, PLT, MPV, RT, MON, NEUT, LYM, EOS, BAS

### Results:

The objective of this study was to determine pharmacokinetics and preliminary toxic effects of W3448-T3U1.E17R-1.uIgG4V9 following single intravenous bolus administrations in naïve female cynomolgus monkeys. Serum drug concentration was showed in FIG. 19, T1/2 was 7.75 h and 19.4 h for 1 mg/kg and 10 mg/kg, respectively. PK parameters were listed in table 13.

### Preliminary Toxic Study in Cynomolgus Monkeys

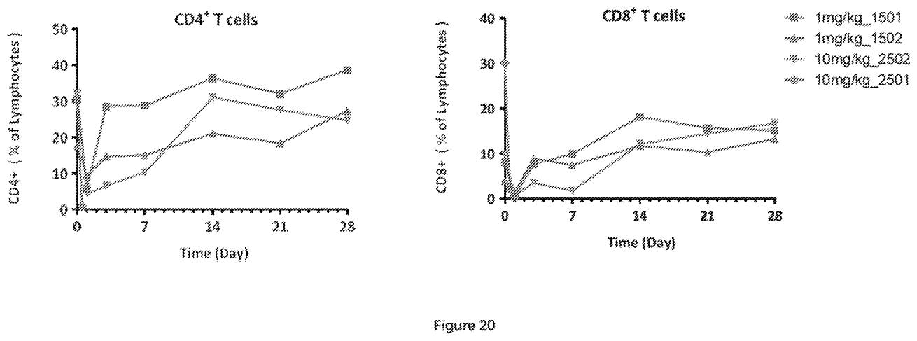

As showed in FIG. 20, at 24 h after dosing, the percentage of CD4+ and CD8+ T cells was decreased in peripheral blood. For 1 mg/kg group, the level of peripheral CD4+ and CD8+ T cell population was fully recovered 3 days post administration; For 10 mg/kg group, T cells were fully recovered 14 days post administration. Dose-dependent effects were observed in the 2 dosing schemes.

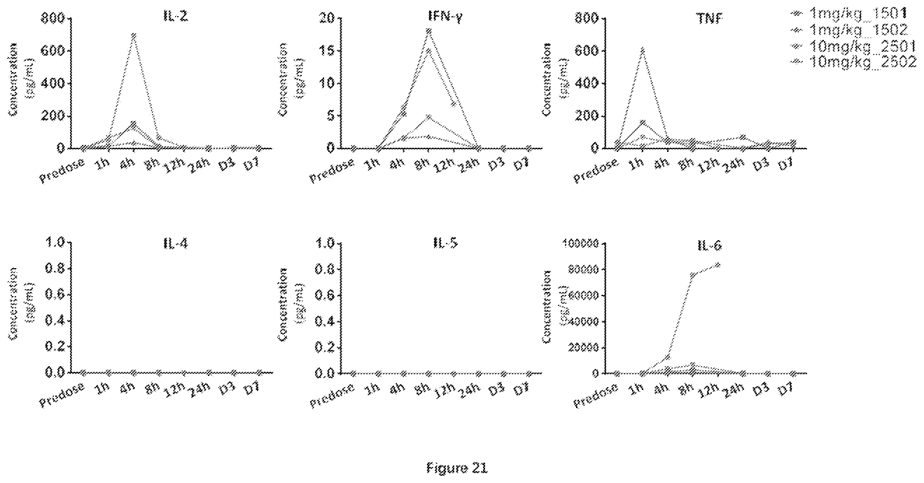

As showed in FIG. 21, no significant change in IL-4 and IL5 concentration was observed after the administration of W3448-T3U1.E17R-1.uIgG4V9. IFN-7 increased slightly in 8 hours after the administration at 1 mg/kg and 10 mg/kg. A transient increase in TNF-α concentration was observed at 1 hour after the administration at 1 mg/kg (C1502).

At 10 mg/kg, one monkey (C2501, female) was found moribund at 8h post injection, with an observed increase in IL-2 concentration at 4 h and very high IL-6 concentration detected 8 h and 12 h post injection, and this monkey was euthanized at 12 h after dosing.

TABLE 13
PK parameters in cynomolgus monkeys

| | W3448-T3U1.E17R-1.uIgG4V9 | | | |
|---|---|---|---|---|
| | 1 mg/kg | | 10 mg/kg | |
| PK parameters | C1501 | C1502 | C2501* | C2502 |
| C₀ (μg/mL) | 4.88 | 5.46 | 91.2 | 162 |
| T_{1/2} (h) | NC | 7.75 | — | 19.4 |
| AUC₀₋ₗₐₛₜ (h*μg/mL) | 35.4 | 30.6 | 1320 | 5981 |
| CL (mL/day/kg) | NC | 399 | — | 39.5 |
| MRT_{0-inf} (h) | NC | 11.2 | — | 32.7 |
| Vss (mL/kg) | NC | 187 | — | 53.9 |

“NC” means not calculated due to less than 3 quantifiable values.
“—” means not applicable.
C2501* was euthanized at 12 h post treatment.

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present disclosure is not limited to the particular embodiments that have been described in detail herein. Rather, reference should be made to the appended claims as indicative of the scope and content of the disclosure.

### REFERENCE

1. Patent: EP 0018795 B1; 1986, 06 26. (OKT3). Monoclonal-antibody-producing hybrid cell line, antibody and method of preparing it, therapeutic composition containing it and its diagnostic and therapeutic uses.
2. Patent No: U.S. Pat. No. 6,217,866 B1, Apr. 17, 2001; Jun. 7, 1995 (Erbitux). Monoclonal antibodies specific to human epidermal growth factor receptor and therapeutic methods employing same.
3. Patent No: U.S. Pat. No. 6,235,883 B1; May 22, 2001; May 5, 1997 (Panitumumab). Human monoclonal antibodies to Epidermal Growth Factor Receptor.
4. WO/2019/057099
5. The EGFR family and its ligands in human cancer: signaling mechanisms and therapeutic opportunities. Y Yarden. European Journal of Cancer 37 (2001) S3-S8
6. Anti-CD3 X Anti-EGFR Bispecific antibody redirect T-cell cytolytic activity to EGFR-positive cancer In vitro and in an animal model. Ursula Reusch, et al. Clin Cancer Res 2006; 12(1).
7. Cytotoxicity of cytokine-induced killer cells targeted by a bispecific antibody to gastric cancer cells. Lin Zhang, et al. Oncology letters 5, 2013
8. Human IgG2 Antibodies against Epidermal Growth Factor Receptor Effectively Trigger Antibody-Dependent Cellular cytotoxicity but, in Contrast to IgG1, Only by Cells of myeloid Lineage. Tanja Schneider-Merck, et al. J Immunol 2010; 184:512-520
9. Targeting the EGFR signaling pathway in cancer therapy. Parthasarathy Seshacharyulu, et al. Expert Opin Ther Targets. 2012 January; 16(1): 15-31
10. Anti-EGFR Therapy: Mechanism and Advances in Clinical Efficacy in Breast Cancer. John F. Flynn, et al. Journal of Oncology Volume 2009
11. EGFR Antagonists in Cancer Treatment. Fortunato Ciardiello, et. al. N Engl J Med 2008; 358:1160-74
12. Emergence of KRAS mutations and acquired resistance to anti-EGFR therapy in colorectal cancer. Sandra Misael, et al. Nature volume 486, Pages 532-536(2012)

1. A bispecific antibody or an antigen-binding portion thereof, comprising a CD3 antigen binding moiety and an EGFR antigen binding moiety,
wherein the CD3 antigen binding moiety comprises a Fab comprising a first VH (VH1) of an anti-CD3 antibody operably linked to a heavy chain CH1 constant region domain, and a first VL (VL1) of the anti-CD3 antibody operably linked to a light chain constant region (CL), and
the EGFR antigen binding moiety comprises a chimeric Fab comprising a second heavy chain variable domain (VH2) of an anti-EGFR antibody operably linked to a first T cell receptor (TCR) constant region (C1), and a second light chain variable domain (VL2) of the anti-EGFR antibody operably linked to a second TCR constant region (C2), and wherein C1 and C2 are capable of forming a dimer via a non-native interchain disulphide bond which is capable of stabilizing the dimer,
wherein:
(A) the CD3 antigen binding moiety comprises:
a heavy chain CDR1 comprising or consisting of an amino acid sequence represented by SEQ ID NO: 1,
a heavy chain CDR2 comprising or consisting of an amino acid sequence represented by SEQ ID NO: 2,
a heavy chain CDR3 comprising or consisting of an amino acid sequence represented by SEQ ID NO: 3,
a light chain CDR1 comprising or consisting of an amino acid sequence represented by SEQ ID NO: 4,
a light chain CDR2 comprising or consisting of an amino acid sequence represented by SEQ ID NO: 5, and
a light chain CDR3 comprising or consisting of an amino acid sequence represented by SEQ ID NO: 6, and
(B) the EGFR antigen binding moiety comprises:
a heavy chain CDR1 comprising or consisting of an amino acid sequence represented by SEQ ID NO: 7,
a heavy chain CDR2 comprising or consisting of an amino acid sequence represented by SEQ ID NO: 8,
a heavy chain CDR3 comprising or consisting of an amino acid sequence represented by SEQ ID NO: 9,
a light chain CDR1 comprising or consisting of an amino acid sequence represented by SEQ ID NO: 10,
a light chain CDR2 comprising or consisting of an amino acid sequence represented by SEQ ID NO: 11, and
a light chain CDR3 comprising or consisting of an amino acid sequence represented by SEQ ID NO: 12.

2. (canceled)

3. The bispecific antibody or the antigen-binding portion thereof of claim 1, wherein
(A) the CD3 antigen binding moiety comprises:
(i) a heavy chain variable domain (VH1) sequence comprising or consisting of SEQ ID NO: 13, and
(ii) a light chain variable domain (VL1) sequence comprising or consisting of SEQ ID NO: 14; and
(B) the anti-EGFR antibody comprises:
(i) a heavy chain variable domain (VH2) sequence comprising or consisting of SEQ ID NO: 15, and
(ii) a light chain variable domain (VL2) sequence comprising or consisting of SEQ ID NO: 16.

4. (canceled)

5. The bispecific antibody or the antigen-binding portion thereof of claim 1, wherein the bispecific antibody or the antigen-binding portion thereof comprises four polypeptide chains:
i) a first heavy chain consisting of VH1-CH1-Hinge1-CH2-CH3, wherein the first heavy chain is represented by SEQ ID NO: 23;
ii) a first light chain consisting of VL1-CL, wherein the first light chain is represented by SEQ ID NO: 22;
iii) a second heavy chain consisting of VH2-C1-Hinge2-CH2-CH3, wherein the second heavy chain is represented by SEQ ID NO: 24, and
iv) a second light chain consisting of VL2-C2, wherein the second light chain is represented by SEQ ID NO: 21,
wherein VH1-CH1 portion of i) and VL1-CL form an anti-CD3 arm, and VH2-C1 of iii) and VL2-C2 form an anti-EGFR arm.

6. (canceled)

7. The bispecific antibody or the antigen binding portion thereof of claim 1, wherein the C1 domain comprises an engineered TCR beta constant region comprising an amino acid sequence of SEQ ID NO: 29; and the C2 domain comprises an engineered TCR alpha constant region comprising an amino acid sequence of SEQ ID NO: 30.

8. (canceled)

9. The bispecific antibody or the antigen-binding portion thereof of claim 1, wherein the Fc region is operably linked to the CH1 domain of the CD3 antigen binding moiety.

10. (canceled)

11. The bispecific antibody or the antigen-binding portion thereof of claim 9, wherein the human Fc region is a human IgG4 or IgG1 Fc region.

12. The bispecific antibody or the antigen-binding portion thereof of claim 1, wherein the bispecific antibody or the antigen-binding portion thereof is a humanized antibody.

13. An isolated nucleic acid molecule, comprising a nucleic acid sequence encoding the bispecific antibody or the antigen-binding portion thereof of claim 1.

14. The isolated nucleic acid molecule of claim 12, wherein the isolated nucleic acid molecule comprises:
a nucleic acid sequence that encodes the heavy chain variable domain (VH1) as set forth in SEQ ID NO: 13,
a nucleic acid sequence that encodes the light chain variable domain (VL1) as set forth in SEQ ID NO: 14,
a nucleic acid sequence that encodes the heavy chain variable domain (VH2) as set forth in SEQ ID NO: 15, and
a nucleic acid sequence that encodes light chain variable domain (VL2) as set forth in SEQ ID NO: 16.

15-21. (canceled)

22. A vector comprising the nucleic acid molecule of claim 13.

23. A host cell comprising the nucleic acid molecule of claim 13.

24. A pharmaceutical composition comprising the bispecific antibody or the antigen-binding portion thereof of claim 1 and a pharmaceutically acceptable carrier.

25. A method for producing the bispecific antibody or the antigen-binding portion thereof of claim 1, comprising the steps of:
expressing the bispecific antibody or the antigen-binding portion thereof in a host cell comprising a nucleic acid sequence encoding the bispecific antibody or the antigen-binding portion thereof; and
isolating the bispecific antibody or antigen-binding portion thereof from the host cell.

26. A method for modulating an immune response in a subject, comprising administering to the subject the bispecific antibody or the antigen-binding portion thereof of claim 1 or a pharmaceutical composition comprising the bispecific antibody or the antigen-binding portion thereof and a pharmaceutically acceptable carrier.

27. A method for inhibiting growth of tumor cells in a subject, comprising administering to the subject an effective amount of the bispecific antibody or the antigen-binding portion thereof of claim 1 or a pharmaceutical composition comprising the bispecific antibody or the antigen-binding portion thereof and a pharmaceutically acceptable carrier.

28. A method for preventing or treating CD3-related and/or EGFR-related diseases in a subject, comprising administering to the subject an effective amount of the bispecific antibody or the antigen-binding portion thereof of claim 1 or a pharmaceutical composition comprising the bispecific antibody or the antigen-binding portion thereof and a pharmaceutically acceptable carrier, wherein the CD3-related and/or EGFR-related diseases are proliferative disorders, immune disorders, or infections.

29. The method of claim 28, wherein the proliferative disorder is cancer selected from colon cancer, lung cancer, liver cancer, cervical cancer, breast cancer, ovarian cancer, pancreatic cancer, melanoma, glioblastoma, prostate cancer, esophageal cancer, or gastric cancer; and wherein the infection is a chronic infection.

30. (canceled)

31. The method of claim 28, wherein the bispecific antibody or antigen-binding portion thereof is administered in combination with a chemotherapeutic agent, radiation and/or other agents for use in cancer immunotherapy.

32-36. (canceled)

37. A kit, comprising a container comprising the bispecific antibody or the antigen-binding portion thereof of claim 1.

38. A host cell comprising the vector of claim 22.


SEQUENCE LISTING (30 sequences)

SEQ ID NO: 1 - protein, 10 aa, Artificial Sequence
  CDR1
GFAFTDYYIH

SEQ ID NO: 2 - protein, 17 aa, Artificial Sequence
  CDR2
WISPGNVNTKYNENFKG

SEQ ID NO: 3 - protein, 10 aa, Artificial Sequence
  CDR3
DGYSLYYFDY

SEQ ID NO: 4 - protein, 17 aa, Artificial Sequence
  CDR1
KSSQSLLNSRTRKNYLA

SEQ ID NO: 5 - protein, 7 aa, Artificial Sequence
  CDR2
WASTRQS

SEQ ID NO: 6 - protein, 8 aa, Artificial Sequence
  CDR3
TQSHTLRT

SEQ ID NO: 7 - protein, 12 aa, Artificial Sequence
  CDR1
GGSVSSGDYYWT

SEQ ID NO: 8 - protein, 16 aa, Artificial Sequence
  CDR2
HIYYSGNTNYNPSLKS

SEQ ID NO: 9 - protein, 9 aa, Artificial Sequence
  CDR3
DRVTGAFDI

SEQ ID NO: 10 - protein, 11 aa, Artificial Sequence
  CDR1
QASQDISNYLN

SEQ ID NO: 11 - protein, 7 aa, Artificial Sequence
  CDR2
DASNLET

SEQ ID NO: 12 - protein, 9 aa, Artificial Sequence
  CDR3
QHFDHLPLA

SEQ ID NO: 13 - protein, 119 aa, Artificial Sequence
  VH
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSS

SEQ ID NO: 14 - protein, 112 aa, Artificial Sequence
  VL
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIK

SEQ ID NO: 15 - protein, 117 aa, Artificial Sequence
  VH
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTV

SEQ ID NO: 16 - protein, 107 aa, Artificial Sequence [sequence omitted]

SEQ ID NO: 17 - DNA, 357 bp, Artificial Sequence
  VH
caggtgcagcttgtgcagtctggggcagaagtgaagaagcctgggtctagtgtcaaggtgtcatgcaaggctagcgggttcgcctttactgactactacatccactgggtgcggcaggctcccggacaagggttggagtggatgggatggatctccccaggcaatgtcaacacaaagtacaacgagaacttcaaaggccgcgtcaccattaccgccgacaagagcacctccacagcctacatggagctgtccagcctcagaagcgaggacactgccgtctactactgtgccagggatgggtactccctgtattactttgattactggggccagggcacactggtgacagtgagctcc

SEQ ID NO: 18 - DNA, 336 bp, Artificial Sequence
  VL
gatatcgtgatgacccagagcccagactcccttgctgtctccctcggcgaaagagcaaccatcaactgcaagagctcccaaagcctgctgaactccaggaccaggaagaattacctggcctggtatcagcagaagcccggccagcctcctaagctgctcatctactgggcctccacccggcagtctggggtgcccgatcggtttagtggatctgggagcgggacagacttcacattgacaattagctcactgcaggccgaggacgtggccgtctactactgtactcagagccacactctccgcacattcggcggagggactaaagtggagattaag

SEQ ID NO: 19 - DNA, 351 bp, Artificial Sequence
  VH
caggtgcaactgcaggaaagcggaccaggacttgtgaagccctctgagactttgtccctgacctgtaccgtctccgggggctctgtcagttcaggggattactactggacatggatcaggcagagtcctgggaaaggcctggagtggattgggcacatctactactcagggaacaccaactacaatcccagcctcaagagcagactgaccatcagcattgacacctccaagacacagttctccctgaagctcagcagcgtgactgccgccgacacagcaatctactattgcgtgcgcgaccgggtgacaggcgcttttgatatttggggccagggcacaatggtcactgtg

SEQ ID NO: 20 - DNA, 321 bp, Artificial Sequence
  VL
gatatccagatgacccagtccccttcctccttgtccgcaagtgtgggagatagagtgaccatcacatgccaggcttctcaggacatctctaactacctgaactggtaccaacagaagcccggcaaggcccctaagctccttatctacgacgcctcaaatctggagaccggagtcccaagcaggttcagcggcagcgggagcgggacagatttcacttttacaattagctccctccagccagaagacattgccacatatttctgtcagcactttgaccatctgcccctggcctttgggggcgggactaaagtggagattaag

SEQ ID NO: 21 - protein, 202 aa, Artificial Sequence
  a second light chain
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 22 - protein, 219 aa, Artificial Sequence
  a first light chain
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 446 aa, Artificial Sequence
  a first heavy chain
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 472 aa, Artificial Sequence
  a second heavy chain
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 25 - DNA, 606 bp, Artificial Sequence
  a second light chain
gatatccagatgacccagtccccttcctccttgtccgcaagtgtgggagatagagtgaccatcacatgccaggcttctcaggacatctctaactacctgaactggtaccaacagaagcccggcaaggcccctaagctccttatctacgacgcctcaaatctggagaccggagtcccaagcaggttcagcggcagcgggagcgggacagatttcacttttacaattagctccctccagccagaagacattgccacatatttctgtcagcactttgaccatctgcccctggcctttgggggcgggactaaagtggagattaagcccgacatccagaaccccgaccccgccgtgtaccagctgagagacagcaagagcagcgacaagagcgtgtgcctgttcaccgacttcgacagccagacccaggtgagccagagcaaggactccgacgtgtatatcaccgacaagtgcgtgctggacatgaggagcatggacttcaagagcaacagcgccgtggcctggagccagaagagcgacttcgcctgcgccaacgccttccagaacagcatcatccccgaggacaccttcttccccagccccgagagcagc

SEQ ID NO: 26 - DNA, 657 bp, Artificial Sequence
  a first light chain
gatatcgtgatgacccagagcccagactcccttgctgtctccctcggcgaaagagcaaccatcaactgcaagagctcccaaagcctgctgaactccaggaccaggaagaattacctggcctggtatcagcagaagcccggccagcctcctaagctgctcatctactgggcctccacccggcagtctggggtgcccgatcggtttagtggatctgggagcgggacagacttcacattgacaattagctcactgcaggccgaggacgtggccgtctactactgtactcagagccacactctccgcacattcggcggagggactaaagtggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 27 - DNA, 1338 bp, Artificial Sequence
  a first heavy chain
caggtgcagcttgtgcagtctggggcagaagtgaagaagcctgggtctagtgtcaaggtgtcatgcaaggctagcgggttcgcctttactgactactacatccactgggtgcggcaggctcccggacaagggttggagtggatgggatggatctccccaggcaatgtcaacacaaagtacaacgagaacttcaaaggccgcgtcaccattaccgccgacaagagcacctccacagcctacatggagctgtccagcctcagaagcgaggacactgccgtctactactgtgccagggatgggtactccctgtattactttgattactggggccagggcacactggtgacagtgagctccgcgtcgaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgaggcagcagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatgccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 28 - DNA, 1416 bp, Artificial Sequence
  a second heavy chain
caggtgcaactgcaggaaagcggaccaggacttgtgaagccctctgagactttgtccctgacctgtaccgtctccgggggctctgtcagttcaggggattactactggacatggatcaggcagagtcctgggaaaggcctggagtggattgggcacatctactactcagggaacaccaactacaatcccagcctcaagagcagactgaccatcagcattgacacctccaagacacagttctccctgaagctcagcagcgtgactgccgccgacacagcaatctactattgcgtgcgcgaccgggtgacaggcgcttttgatatttggggccagggcacaatggtcactgtgctggaggacctgaagaacgtgttccctcccgaggtggccgtgttcgaacccagcgaggccgagatcagccacacccagaaggccaccctggtgtgtctggccaccggcttctaccccgaccacgtggagctgagctggtgggtgaacggcaaggaggtgcacagcggcgtgtgtaccgaccctcagcccctgaaggagcagcccgccctgcaggacagcaggtacgccctgagcagcaggctgagagtgagcgccaccttctggcagaaccccaggaaccacttcaggtgccaggtgcagttctacggcctgagcgagaacgacgagtggacccaggacagggccaagcccgtgacccagatcgtgagcgctgaggcctggggcagatatggtcccccatgcccaccatgcccagcacctgaggcagcagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtgcaccctgcccccatcccaggaggagatgaccaagaaccaggtcagcctgagctgcgcggtcaaaggcttctaccccagcgacatcgccgtggagtgggagagcaatgggcagccggagaacaactacaagaccacgcctcccgtgctggactccgacggctccttcttcctcgttagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 29 - protein, 129 aa, Artificial Sequence
  TCR beta constant domain
LEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGR

SEQ ID NO: 30 - protein, 95 aa, Artificial Sequence
  TCR alpha constant domain
PDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS